(19) United States
(12) Patent Application Publication
(10) Pub. No.: US 2026/0265693 A1
(43) Pub. Date: Sep. 10, 2026

(54) CONSTITUTIVE CYTOKINE RECEPTORS

(71) Applicant: QUELL THERAPEUTICS LIMITED, London (GB)

(72) Inventors: Marc MARTINEZ-LLORDELLA, London (GB); Sim TUNG, London (GB); Bethany MCCLOSKEY, London (GB)

(21) Appl. No.: 18/723,763

(22) PCT Filed: Dec. 22, 2022
(86) PCT No.: PCT/GB2022/053359

§ 371 Date: Jun. 24, 2024

(30) Foreign Application Priority Data
Dec. 22, 2021 (GB) 2118797.6

(51) Int. Cl. C12N 5/0783; A61K 40/11; A61K 40/31; A61K 40/42; C07K 14/71; C07K 16/18; C07K 16/28

(52) U.S. Cl. CPC C12N 5/0637; A61K 40/11; A61K 40/31; A61K 40/42; A61K 40/421; C07K 14/71; C07K 16/18; C07K 16/2833; C12N 2501/14; C12N 2510/00

(57) ABSTRACT

There is provided herein a recombinant protein comprising an exodomain, at least a portion of which is derived from the extracellular region of EPOR, wherein the exodomain comprises a dimerisation domain allowing dimerisation of said recombinant protein with a second protein and provision of a signal into said T cell in the absence of a signal inducer molecule. Nucleic acid molecules encoding such a recombinant protein, recombinant constructs, vectors and cells containing the nucleic acid molecule, methods of producing such cells, and therapeutic uses thereof are also provided.

### FIELD

The present disclosure and invention relate to a recombinant protein and its use in adoptive cell therapy (ACT). In particular, the recombinant protein can provide a cell expressing the protein with a desired constitutive signal, e.g., a constitutive STAT-mediated signal. The signal may confer a desired effect or property on the cell, e.g., increased function, activity, vitality, or survival, e.g., persistence in a transplanted host subject. Also provided are nucleic acid molecules encoding such a recombinant protein, recombinant constructs, vectors and cells containing the nucleic acid molecule, methods of producing such cells, and therapeutic uses thereof.

### BACKGROUND

Adoptive cell therapy (ACT), that is the administration of functional immune cells to a subject, has become an established and evolving immunotherapeutic approach for various medical conditions, including notably malignant or infectious diseases. Tumour-infiltrating lymphocytes were initially shown to be effective in treating metastatic melanoma, and subsequently re-directed T-cells or NK cells expressing chimeric antigen receptors (CARs) or heterologous T-cell receptors (TCRs) to target different cellular target molecules have been developed and adopted for clinical use. Initial approaches used immune cells with cytotoxic properties, e.g. cytotoxic T-cells or NK cells, to target and kill unwanted or deleterious cells in the body, but more recently regulatory T cells (Tregs) have been developed for ACT. Tregs have immunosuppressive function. They act to control cytopathic immune responses and are essential for the maintenance of immunological tolerance. The suppressive properties of Tregs can be exploited therapeutically, for example to improve and/or prevent immune-mediated organ damage in inflammatory disorders, autoimmune diseases and in transplantation.

To be useful in ACT, the transplanted, or administered, cells need to survive and persist in the recipient (the subject of the ACT therapy) in a functional state long enough to exert a useful therapeutic effect. Further, to be prepared in sufficient numbers for therapeutic use, the cells need to be generated (e.g. engineered), cultured and expanded in vitro.

The growth factor interleukin-2 (IL-2) is essential for the homeostasis of immune cells, including notably Tregs (generation, proliferation, survival), as well as for their suppressive function and phenotypic stability. Activated conventional T cells (Tcons) are the main source of IL-2 in vivo. Tregs, in contrast, cannot produce IL-2 and depend on paracrine access to IL-2 produced by Tcons present in the microenvironment.

The availability of IL-2 has a critical impact on the therapeutic effects of Tregs expanded in vitro and transferred into patients. This is due to the following: 1) in vitro expansion protocols typically require high concentrations of IL-2, which renders Tregs highly dependent on this cytokine; 2) the concentration of IL-2 is often reduced in patients as a result of the administration of immunosuppressive drugs; and 3) within the inflamed tissue microenvironment access to IL-2 is often limited. Liver transplantation constitutes a particularly challenging indication, given that the levels of IL-2 in the inflamed liver are known to be reduced, which is further aggravated by the routine use of calcineurin inhibitors, which substantially decrease the capacity of Tcons to produce IL-2. The administration of low doses of exogenous IL-2 restores the Treg dysfunction induced by calcineurin inhibitors and promotes the accumulation of Tregs in the liver. However, a concern with the therapeutic use of low-dose IL-2 is the risk of simultaneously activating Tcons, which can enhance tissue damage.

In WO 2020/044055 an approach is described to circumvent the need to administer exogenous IL-2. In this case Treg cells are engineered to express a CAR which has been modified such that it is capable of providing a productive IL-2 signal to the cell upon binding to its target antigen. In other words, the intracellular signalling domain of the CAR, the endodomain, includes sequences, or domains, derived from IL receptors, which allow it to transmit an “IL-2 signal” in the absence of endogenous IL-2, and without the need for IL-2 binding. IL-2 signals through the transcription factor STAT5 (Signal Transducer and Activator of Transcription 5), which is phosphorylated in its active state by the kinases JAK1 and/or JAK2, which are normally activated when interleukins (e.g. IL-2) bind to their receptors.

Accordingly, the CAR in WO 2020/044055 comprises an endodomain which comprises a STAT5 association motif and a JAK1- and/or a JAK2-binding motif.

Analogously, other immune cells for ACT, e.g. cytotoxic T-cells, or other Teffector cells, including CAR-T cells, may also require or benefit from additional signalling capacity being provided to the cell to increase survival or persistence of function. Accordingly, the need for additional signalling, or more particularly engineered signalling, whether to increase survival or persistence, or to improve the functional activity or therapeutic effect of cells for ACT, is not limited to Treg cells.

Whilst WO 2020/04405 provides an important advance, there is a continuing need in the field of ACT for new and improved approaches, and in particular approaches which avoid or reduce the need to develop a modified CAR for each target, and which may have a more universal application.

Erythropoietin (EPO) is a glycoprotein made by the fetal liver, or in adults by kidney perivascular interstitial fibroblasts. EPO production is primarily stimulated in vivo by tissue hypoxia which results in stabilisation of hypoxia inducible factor (HIF)-1α transcription factor and the subsequent transcription of EPO. EPO induces erythropoiesis by binding to the Erythropoietin Receptor (EPOR) on erythroid precursor cells but is additionally expressed in non-hematopoietic tissues. EPOR is a 508 amino acid transmembrane receptor, including an extracellular domain containing a WSXWS motif, a single transmembrane hydrophobic region and a cytoplasmic domain. EPOR forms a homodimer upon EPO binding and is capable of signalling through activation of JAK2, MAPK and PI3 kinases and STAT5 phosphorylation. It has also been suggested in the literature that EPOR may be capable of heterodimerisation with CD131.

WO2019/169290 described an approach to inducibly introduce a signal to a cell using chimeric receptors comprising dimerization domains capable of dimerising in the presence of an agent. Certain constructs disclosed in WO2019/169290 comprised domains from EPOR or from IL2RB but signalling could only be induced in the presence of a ligand. Whilst inducibility may allow control of signalling within a cell and may have utility in certain disease conditions, in conditions where ligand may be absent or present at low levels, signalling from such constructs may be reduced, resulting in lack of persistence of the cell.

There is thus a need to develop cytokine receptors which are capable of providing a constitutive signal to a cell, which have utility in conditions where inducibility is not possible and/or desirable.

### SUMMARY

The present inventors have identified a recombinant protein which is capable of homodimerization and of providing a constitutive signal to a cell expressing the recombinant protein in the absence of a ligand. Particularly, the recombinant protein has an exodomain derived from a modified extracellular domain of a cytokine receptor, wherein the modification promotes homodimerisation and constitutive signalling in the absence of natural ligand for the cytokine receptor. The recombinant protein particularly may additionally comprise an endodomain and can be used in several cell types to provide a consistent signal, e.g., STAT signal, which can allow cells, particularly in the context of ACT, to survive in environments where cytokine receptor ligand is or may be limited. In such environments, the use of an inducible protein to provide a survival signal to a cell may not be appropriate and the present invention has particular utility here, addressing this particular problem.

Particularly, the recombinant protein identified for use by the inventors may comprise an exodomain which is derived from the extracellular region of EPOR, but additionally comprises a modification allowing constitutive signalling through the receptor in the absence of EPO. The extracellular region may also retain its ability to bind EPO if present in the environment, potentially providing a boost to the signalling through the modified cytokine receptor. Thus, advantageously, the recombinant protein is capable of dimerising and providing a constitutive signal to a cell and may also be capable of binding environmental ligand when available, creating the possibility of enhanced signalling. However, the constitutive signalling alone in the absence of ligand is sufficient to provide a cell expressing the recombinant protein with the desired functionality/survival advantage. In one embodiment, the recombinant protein may comprise a modified human extracellular region of EPOR having an amino acid substitution at residue 154 to cysteine, which promotes homodimerization of monomeric recombinant protein chains in the absence of EPO and constitutive signalling.

Although the recombinant protein for use in the invention may provide any signal into a cell expressing the recombinant protein, it is particularly envisaged that the recombinant protein may be utilised to enable cells, particularly in the context of a cell therapy, to survive or persist after administration to a subject, to provide the best opportunity for a therapy to be efficacious in a diseased subject. Particularly, the signalling through the recombinant protein involves tyrosine kinase activity, and protein phosphorylation, and more particularly, the signalling involves Janus kinase (JAK) phosphorylation and activity, e.g., activation of the JAK-STAT signalling pathway involving JAK1 and/or JAK2. Thus, particularly the endodomain of the recombinant protein discussed herein may be derived or partially derived from the same protein as the exodomain (EPOR, which comprises a JAK2 binding motif and a STAT5 association motif) or may be from one or more different proteins (the recombinant protein may be a chimeric recombinant protein), where the different protein(s) allow for transduction of the same or similar signal as EPOR or may potentially allow for the provision of a different cellular signal from the natural EPOR receptor. Advantageously, when the endodomain of the recombinant protein is derived from EPOR, the inventors have further identified that it is possible to utilise a modified endodomain, which has a reduced ability to bind to SHP1 which once activated can inhibit JAK2 phosphorylation and also has the ability to impact other endogenous receptors such as IL2R. In addition, when the endodomain of the recombinant protein is derived from EPOR, the inventors have further identified that the insertion of an amino acid sequence, for example a cytoplasmic tail at the C-terminus of the part of the endodomain that is derived from the endodomain of EPOR, may increase or stabilise cell surface expression of the recombinant protein and/or may increase sensitivity to EPO.

The recombinant protein described herein has particular utility in T cells which may be required to persist for periods of time within a subject after administration and more particularly has utility in Tregs which require STAT5 signalling to survive (usually through their endogenous IL2R) and are completely reliant on supply of exogenous ligand to trigger such signalling. The use of a recombinant protein within Tregs is particularly advantageous given the constitutive signalling ability of the receptor, the need of Tregs for constant STAT5 signalling and the lack of naturally available ligand (e.g. IL2) within particular disease conditions (e.g., Type 1 diabetes). The inventors have advantageously shown that Tregs expressing the recombinant proteins described herein maintain their usual phenotype and suppressive function, and thus the stability of the cell may be retained.

Accordingly, the present invention provides a T cell comprising a recombinant protein, wherein said recombinant protein comprises an exodomain, at least a portion of which is derived from the extracellular region of EPOR wherein the exodomain comprises a dimerisation domain allowing dimerisation of said recombinant protein with a second protein and provision of a signal into said T cell in the absence of a signal inducer molecule.

The dimerisation domain can be any dimerisation domain which allows binding of the recombinant molecule to a second protein in the absence of a signal inducer molecule and signalling into the cell. The dimerisation domain should thus allow the provision of a constitutive signal to the cell. The dimerisation domain may be outside of the portion of the exodomain derived from the extracellular region of EPOR (i.e., in a different position in the exodomain to this portion) or particularly, may be comprised within the portion of the exodomain which is derived from the extracellular region of EPOR.

Accordingly, in this respect, the present invention provides a T cell comprising a recombinant protein, wherein said recombinant protein comprises an exodomain, at least a portion of which is derived from the extracellular region of EPOR, wherein said portion comprises a modification relative to the extracellular region of EPOR allowing dimerization of said recombinant protein with a second protein and provision of a signal into said T cell in the absence of a signal inducer molecule.

Thus, the recombinant protein present within the T cell may be expressed in the T cell from a nucleic acid molecule which has been transduced into the cell, or into a precursor cell, or may be expressed from an endogenous nucleic acid sequence which has been modified using gene editing technology.

In a particular embodiment, the recombinant protein comprises an exodomain which allows disulphide-linked dimerisation, of the recombinant protein with a second protein. Thus, the recombinant protein comprises at least one dimerisation domain which particularly allows disulphide-linked dimerisation.

It will further be appreciated by a skilled person that the dimerization may be homodimerization, where the recombinant protein binds to another recombinant protein as described herein, or heterodimerisation, where the recombinant protein dimerises with a different protein. It will be appreciated that for heterodimerisation to occur the second protein should comprise a cognate dimerisation domain which is capable of binding to the dimerisation domain present within the recombinant protein. It is typically the dimerization of the recombinant protein that allows the production of a signal to the T cell. In a particular embodiment of the invention, the dimerisation is homodimerization and in this respect, the present invention provides a T cell comprising a recombinant protein, wherein said recombinant protein comprises an exodomain, at least a portion of which is derived from the extracellular region of EPOR, wherein said exodomain comprises a dimerisation domain allowing homodimerization of said recombinant protein and provision of a signal into said T cell in the absence of a signal inducer molecule. Particularly, as discussed above, the at least a portion of the exodomain derived from the extracellular region of EPOR may comprise a modification relative to the extracellular region of EPOR which may allow homodimerization of said recombinant protein and provision of a signal into said T cell in the absence of a signal inducer molecule.

The dimerization of the recombinant protein occurs spontaneously in the present of a second protein and does not require the presence of a signal inducer molecule, e.g., erythropoietin and more particularly any signal inducer molecule (including any ligand or dimerization inducer molecule).

In this respect, the present invention further provides a T cell comprising a recombinant protein, wherein said recombinant protein comprises an exodomain, at least a portion of which is derived from the extracellular region of EPOR, wherein the exodomain comprises a dimerisation domain allowing dimerization of said recombinant protein with a second protein and provision of a signal into said T cell in the absence of EPO. Particularly, as discussed above, the at least a portion of the exodomain derived from the extracellular region of EPOR may comprise a modification relative to the extracellular region of EPOR which may allow homodimerization of said recombinant protein and provision of a signal into said T cell in the absence of EPO.

Particularly, the invention provides a T cell comprising a recombinant protein, wherein said recombinant protein comprises an exodomain, at least a portion of which is derived from the extracellular region of EPOR, wherein the exodomain comprises a dimerisation domain allowing homodimerization of said recombinant protein and provision of a signal into said T cell in the absence of EPO. Particularly, as discussed above, the at least a portion of the exodomain derived from the extracellular region of EPOR may comprise a modification relative to the extracellular region of EPOR which may allow homodimerization of said recombinant protein and provision of a signal into said T cell in the absence of EPO.

The portion of the exodomain derived from the extracellular region of EPOR may comprise the full-length extracellular region of EPOR, or a portion or variant sequence thereof. Particularly, the EPOR extracellular region sequence present may comprise at least one modification which allows dimerization of the recombinant protein and constitutive signalling in the absence of a signal inducer molecule, particularly EPO. Thus the at least one modification(s) may be any modification(s) which results in constitutive signalling of the recombinant protein. The EPOR extracellular region sequence present within the recombinant protein may comprise other modifications in addition to the at least one modification allowing dimerization of the recombinant protein and the provision of a constitutive signal in the absence of a signal inducer molecule, particularly EPO.

Particularly, the portion of the exodomain derived from the extracellular region of EPOR may be derived from SEQ ID NO. 1, more particularly from amino acids 1-250 of SEQ ID NO. 1 (SEQ ID NO. 5). The at least one modification may be a mutation to the amino acid sequence of the extracellular region of EPOR, allowing dimerisation, particularly disulphide-linked dimerisation and/or maybe the insertion of a sequence allowing dimerisation in the absence of a signal inducer molecule, e.g. a leucine zipper. More particularly the recombinant protein comprises a modification of the arginine at position 154 to cysteine (R154C) of SEQ ID NO. 5. In a most particular embodiment, the exodomain may comprise or consist of the amino acid sequence of SEQ ID NO. 2 or SEQ ID NO. 4.

Thus, in another embodiment, the present invention provides a T cell comprising a recombinant protein, wherein said recombinant protein comprises an exodomain comprising the extracellular region of EPOR or a portion or variant thereof, which has been modified to allow dimerization of said recombinant protein with a second protein and to provide a signal in said T cell in the absence of a signal inducer molecule, particularly EPO.

Portions or variants of the extracellular region of EPOR may in one embodiment retain the function of the EPOR extracellular region and thus be capable of binding to EPO. In this way, as discussed previously, it may be possible to provide an increase to the signal provided to the cell in the presence of EPO. Alternatively, or additionally, such portions or variants may have at least 70, 80 or 90% identity to the EPOR extracellular region. In a particular embodiment, variants and portions may comprise at least one modification providing a dimerisation domain, allowing dimerization of the recombinant protein with a second protein and the provision of a signal to an expressing cell in the absence of a signal inducer molecule, e.g. EPO.

In a further embodiment, the invention provides a T cell comprising a recombinant protein, wherein said recombinant protein comprises an exodomain comprising SEQ ID NO. 2 or SEQ ID NO. 4, or a portion or a variant thereof having at least 90% identity to SEQ ID NO. 2 or SEQ ID NO. 4 and comprising a cysteine at position 130 of SEQ ID NO. 2 or a cysteine at position 154 of SEQ ID NO. 4, wherein said recombinant protein is capable of dimerising with a second protein and of providing a signal in said T cell in the absence of a signal inducer molecule, particularly EPO.

The exodomain of the recombinant protein may comprise additional heterologous domains or regions not associated with dimerisation (i.e. non-dimerising domains or regions which are not present in wildtype EPOR) in addition to the portion which is derived from the extracellular region of EPOR, e.g., suicide motifs or tags and thus the exodomain may not consist solely of the portion derived from the extracellular region of EPOR.

As discussed above, the exodomain of the recombinant protein as described herein comprises at least one dimerisation domain (e.g. at least 2, 3, 4, or 5) dimerisation domains allowing dimerisation and constitutive signalling in the absence of a signal inducer molecule. Particularly, the dimerisation domains may be introduced into the portion of the exodomain derived from the extracellular region of EPOR e.g. by making one or more modifications to the amino acid sequence, e.g. at least 2, 3, 4, or 5 modifications). Thus, a signal inducer molecule (e.g. EPO) is not required for signalling to occur in a cell comprising a recombinant protein as described herein. Particularly, the recombinant protein is capable of dimerising and of providing a constitutive signal in the absence of EPO, the natural ligand for the EPOR. Although therefore not required for constitutive signalling, the exodomain of the recombinant protein described herein may comprise one or more dimerisation domains allowing inducible dimerisation of the recombinant protein with a second protein. Such “inducible dimerisation domains” require the presence of a dimerisation inducer molecule to dimerise. In one embodiment, the presence of at least one inducible dimerisation domain(s) may provide for enhanced or increased signalling through the recombinant protein as discussed in detail below. Typically, the one or more inducible dimerisation domains may be heterologous to EPOR (i.e. may not occur in wildtype EPOR). It will be appreciated by a skilled person that the one or more inducible dimerisation domains may be comprised anywhere within the recombinant protein (including within any endodomain or exodomain) but particularly may be comprised within the exodomain, e.g. within the portion of the exodomain derived from the extracellular region of EPOR or within the exodomain but not within the portion derived from the extracellular region of EPOR, e.g. as a separate domain or region.

The recombinant protein of the invention may comprise a transmembrane domain to anchor the exodomain within the cell membrane. The transmembrane domain may be derived from any protein which has a transmembrane domain, including for example EPOR, IL2RB, CD28, CD8 etc. In one embodiment, the transmembrane domain may associate with the transmembrane domain of another protein, for example, the transmembrane domain may be from a TREM protein which is capable of associating with DAP10/12.

The signal which may be provided by the recombinant protein may be a signal which improves or increases a functional property or activity of the cell. Thus, the function or effect of a cell may be increased, which may be a function or effect in vitro or in vivo, that is during generation or expansion of a cell which is being prepared for ACT, or once the cell has been administered to a subject. This may be, for example cell survival, persistence of the cell, persistence of function of the cell, vitality, functional effect (e.g., immunosuppressive or cytotoxic effect), phenotype of the cell, including memory phenotype, proliferation capacity and/or therapeutic efficacy of the cell. The increase may be seen in a cell which comprises the recombinant protein relative to a cell which does not comprise the protein. The signal as discussed above, is particularly a constitutive signal.

In a particular embodiment the signal is a pro-survival signal, which helps the cell to survive and to maintain its ability to function during and after culture, and to persist and maintain its functional ability following administration to a subject in the course of therapy. It may alternatively be referred to as a persistence signal. Thus, the recombinant protein may be expressed in a cell to impart an inducible pro-survival signalling capacity to the cell. It has particular utility in cells prepared for use in ACT therapy and may be expressed in such cells together with an antigen receptor, such as a TCR, or a CAR, or any chimeric receptor. The protein thus has utility in the engineering of cells for ACT.

In an embodiment the signal is a STAT-mediated signal (e.g., a STAT3 or a STAT5 mediated signal), and more particularly, a STAT5-mediated signal, which can normally be induced in a cell by interleukins such as IL-2, or by EPO.

The recombinant protein of the invention particularly comprises an endodomain or is capable of associating with a further signalling protein comprising an endodomain to provide a signal to the cell. Thus, the endodomain may be part of the recombinant protein as described herein or for example may be comprised within a signalling protein that associates with the recombinant protein, e.g., through their respective transmembrane domains, e.g. where the transmembrane domain of the recombinant protein may be from a myeloid receptor and the transmembrane domain of the signalling protein may be derived from DAP10 or DAP12.

In a particular embodiment, the endodomain comprises at least one JAK 1 and/or JAK2 binding motif and at least one STAT association motif (e.g., STAT3 and/or STAT5 association motif). Particularly, the endodomain may comprise at least one JAK2 binding motif and at least one STAT5 association motif, or at least one JAK1 binding motif and at least one STAT5 association motif. The endodomain may comprise further domains, e.g., may comprise at least one JAK 3 binding motif.

The present invention further provides a T cell comprising a nucleic acid molecule (or alternatively termed, a polynucleotide) comprising a nucleotide sequence which encodes a recombinant protein as defined herein.

The nucleic acid molecule may be in the form of a construct, or more particularly, a recombinant construct, comprising the nucleic acid molecule and one or more other nucleotide sequences (a nucleotide sequence of interest). For example, the construct may comprise the nucleic acid molecule and a regulatory sequence, e.g., an expression control sequence, and/or a sequence encoding another functional protein (or more generally, a protein of interest), for example a receptor, e.g., a CAR or TCR etc. Where the endodomain is present within a further signalling protein, nucleotide sequences encoding the recombinant protein and the signalling protein may be provided in the same construct. Alternatively, a separate nucleic acid molecule or construct may be provided for the recombinant protein and the signalling protein. The construct may comprise one or more co-expression sequences linking the nucleic acid molecule with one or more other coding nucleotide sequences.

The nucleic acid molecule or construct as defined herein may be comprised within a vector. Where separate recombinant protein and other expressed proteins (e.g., functional proteins or signalling proteins) are encoded in separate molecules or constructs, they may each be contained in a separate vector. There may accordingly be a set of vectors, each comprising a sequence encoding a separate desired protein.

The vector may be a viral or non-viral vector. In an embodiment the vector may comprise a nucleic acid molecule as defined herein and a further nucleotide sequence encoding a protein of interest, notably a receptor, e.g., a CAR or TCR.

The cell (T cell) expresses the recombinant protein on its cell membrane.

Also provided according to the invention is a cell population comprising a cell as defined herein, e.g., a T cell population.

In an embodiment, the cell is a T cell, including CD4+ or CD8+ T cells or any precursor thereof. The cell may therefore be a stem cell, or more particularly a haemopoietic stem cell (HSC) or pluripotent stem cell (PSC), e.g. an induced pluripotent stem cell (iPSC), i.e. a cell prior to differentiation or conversion to a T cell. Particularly, the T-cell may be a Treg cell. The cell may be a primary cell or from a cell line.

The invention further provides a method of preparing a T cell as defined herein (i.e. a cell comprising the recombinant protein or a nucleic acid molecule encoding the recombinant protein), said method comprising introducing into a T cell (e.g. transducing or transfecting a cell with), a nucleic acid molecule, construct or vector as defined herein. The method may include allowing the recombinant protein to be expressed in the cell. This may include, for example, culturing the cell.

Such a method may further comprise a preceding step of isolating, enriching providing or generating a cell to be used in the method. Further, a cell may be isolated or enriched or generated after the step of introducing the nucleic acid molecule. For example, the nucleic acid molecule may be introduced into a precursor or progenitor cell, e.g. a stem cell, and the cell may then be induced or caused to differentiate, or change, into a T cell. For example, an iPSC cell may be differentiated into a Treg or other T cell or a Tcon cell may be converted into a Treg cell, etc.

This aspect may also include a method of preparing a recombinant protein as defined herein, said method comprising introducing into a T cell, a nucleic acid molecule, construct or vector as defined herein, allowing the chimeric protein to be expressed by the cell, and optionally detecting and/or collecting the chimeric protein.

The invention additionally provides a method of preparing a T cell as defined herein comprising genetically engineering the endogenous nucleic acid encoding the EPOR to comprise a modification so that the expressed EPOR comprises an extracellular region which homodimerizes in the absence of EPO, thus providing a signal to the T cell. Particularly, the method may be carried out using T cell precursors, such as iPSCs prior to differentiation to T cells, and more particularly, the expressed EPOR from the genetically engineered endogenous nucleic acid comprises an amino acid substitution at position 154 from R to C.

The invention further provides a method of promoting the survival or persistence of a cell, said method comprising introducing into the cell, a nucleic acid molecule, construct or vector as defined herein, or by genetic engineering endogenous nucleic acids as described above.

This aspect may comprise an additional step of administering a cell as defined herein to a subject, and optionally administering a signal inducer molecule to the subject (e.g., EPO, where a boost to the constitutive signal is desired). Any inducer may be administered before, during or after administration of the cell. Thus, in this aspect, the method may be carried out in vivo. Alternatively, the method may be carried out in vitro/ex vivo.

Alternatively viewed, the invention further provides use of a recombinant molecule as described herein for promoting the survival or persistence of a cell expressing said recombinant molecule.

As noted above, the recombinant protein may advantageously be expressed in a cell in the context of therapy. Whilst the cell may be an unmodified cell, in the sense of not being further genetically engineered for therapeutic use, for example a T cell isolated from a subject, or a cell derived from such an isolated cell (although of course the cell would be modified by the present method to express the recombinant protein), typically the cell will be a cell which is additionally modified, or engineered to express a further molecule (i.e. a further protein), notably a receptor, e.g. a CAR or TCR.

Thus, the invention further provides a method of preparing a cell for use in adoptive cell transfer therapy (ACT), said method comprising providing said cell with a recombinant protein as defined herein. More particularly, this method may comprise introducing into said cell a nucleic acid molecule, construct or vector as defined herein. The method may also comprise introducing into the cell a separate nucleic acid molecule, construct or vector, for example which comprises a nucleotide sequence which encodes a separate (e.g. second) protein, or a therapeutic protein, notably a receptor, e.g. a CAR or TCR.

Additionally, the invention provides a pharmaceutical composition comprising a cell, cell population or a vector comprising a nucleic acid molecule encoding a recombinant protein as defined herein, together with at least one pharmaceutically acceptable carrier or excipient. In an embodiment the cell or the vector comprises an additional nucleotide sequence encoding a further protein, notably a chimeric protein, or a receptor, e.g. a CAR or TCR. In another embodiment the cell comprises a separate nucleic acid molecule, construct or vector which comprises a nucleotide sequence which encodes a further protein, notably a chimeric protein, or a receptor, e.g. a CAR or TCR.

Further, the invention provides a cell or cell population comprising a nucleic acid molecule encoding a recombinant protein as defined herein, or a pharmaceutical composition of as defined herein, or a vector comprising a nucleic acid molecule encoding a recombinant protein as defined herein for use in therapy. Particularly, the cell, cell population or a pharmaceutical composition comprising the cell or cell population may be for ACT. The vector or pharmaceutical composition comprising the vector may be for gene therapy. The ACT or gene therapy may be for the treatment or prevention of any condition which is responsive to ACT or gene therapy, in particular immunotherapy by ACT or gene therapy.

The invention further provides a cell, cell population, vector comprising a nucleic acid molecule encoding a recombinant protein as defined herein or pharmaceutical composition as defined herein for use in the treatment of or prevention of cancer, or an infectious, neurodegenerative, inflammatory, autoimmune or allergic disease or any condition associated with an unwanted or deleterious immune response. In particular, where the cell is a Treg or other immunosuppressive cell, the cell may be used for inducing immunosuppression (i.e. for suppressing an unwanted or deleterious immune response), for example to improve and/or prevent immune-mediated organ damage in inflammatory disorders, autoimmune or allergic diseases or conditions, and in transplantation.

This aspect also provides a method of adoptive cell transfer therapy, said method comprising administering to a subject in need of said therapy a cell, or cell population comprising a nucleic acid molecule encoding a recombinant protein as defined herein, or a pharmaceutical composition as defined herein, particularly an effective amount of said cell, cell population or pharmaceutical composition.

Also provided is a method of treating or preventing cancer, or an infectious, neurodegenerative, inflammatory, autoimmune or allergic disease or a condition associated with an unwanted or deleterious immune response, said method comprising administering to a subject in need thereof a cell, cell population, or vector comprising a nucleic acid molecule encoding a recombinant protein as defined herein or a pharmaceutical composition as defined herein, particularly an effective amount of said cell, cell population, vector or pharmaceutical composition.

Further, there is provided use of a cell, cell population or vector comprising a nucleic acid molecule encoding a recombinant protein as defined herein in the manufacture of a medicament for use in treating or preventing cancer, or an infectious, neurodegenerative, inflammatory, autoimmune or allergic disease or a condition associated with an unwanted or deleterious immune response.

In some embodiments of these therapeutic aspects the use may be in induction of tolerance to a transplant; treating and/or preventing cellular and/or humoral transplant rejection; treating and/or preventing graft-versus-host disease (GvHD), an autoimmune or allergic disease; to promote tissue repair and/or tissue regeneration; or to ameliorate inflammation. In particular, in such embodiments the cell may be a Treg cell.

In the various therapeutic aspects set out above the cell, cell population, vector or pharmaceutical composition may be for use in combination with, or together with, a signal inducer molecule (particularly EPO).

Accordingly, a further aspect provides a combination product comprising (a) a cell, cell population, or vector comprising a nucleic acid molecule encoding a recombinant protein as defined herein or a pharmaceutical composition as defined herein, and (b) a signal inducer molecule (particularly EPO), for use in therapy, particularly ACT or gene therapy.

The therapy may be any therapy as defined above, and further described herein.

The components (a) and (b) of the combination product may be for separate, sequential or simultaneous use.

### DESCRIPTION OF THE FIGURES

FIG. 1 depicts a wildtype murine EPOR showing the homodimerization of EPOR chains in the presence of EPO. When the receptor has been activated by EPO, JAK2 is able to associate with the endodomain of the receptor, resulting in the phosphorylation of Y343 and the recruitment and dimerisation of STAT5. SHP1 provides a negative feedback loop for control of signalling through the EPOR and can directly inhibit JAK2.

FIG. 2 shows different recombinant proteins as described herein. FIG. 2A shows a recombinant protein comprising an exodomain from EPOR with a modification at position 129 to cysteine (murine sequence numbering), the transmembrane domain of EPOR and the endodomain of EPOR comprising Y343 (murine sequence numbering). The recombinant protein is capable of homodimerization and of providing a constitutive signal to a cell through STAT5. FIG. 2B shows a recombinant protein comprising an exodomain from EPOR with a modification at position 129 to cysteine (murine sequence numbering), the transmembrane domain of EPOR and a truncated endodomain of EPOR, removing the binding site for SHP1 and thus removing the negative feedback of SHP1 on JAK2, enhancing STAT5 signalling.

FIG. 2C shows a recombinant protein comprising an exodomain from EPOR with a modification at position 129 to cysteine (murine sequence numbering), the transmembrane domain of EPOR and an endodomain comprising a truncated endodomain from IL2RB which retains a JAK1 binding motif and a STAT5 association motif (with Y510).

FIG. 3 shows the constructs tested in Example 5. An exodomain derived from the EPOR exodomain allowed cells expressing the protein to be identified using EPOR antibody.

FIG. 4A provides FACS plots showing the transduction efficiencies of fresh Tregs transduced with various constructs. Transduction efficiencies were measured using an HLA-A2 dextramer and EPOR expression.

FIG. 4B provides FACS plots showing the transduction efficiencies of frozen Tregs transduced with various constructs. Transduction efficiencies were measured using an HLA-A2 dextramer and EPOR expression.

FIG. 5 shows pSTAT5 MFI for transduced and untransduced fractions of cells with and without EPO treatment.

FIG. 6A shows the proportion (%) of transduced cells (determined by presence of A2 CAR) in a population of cells over 6 days with or without IL2.

FIG. 6B shows FoxP3 expression of transduced (A2 Dex+) and untransduced (A2 Dex−) fractions of cells over 6 days with or without IL2.

FIG. 6C shows fold expansion of Tregs transduced with either the 658 or 786 construct over 5 days and 7 days.

FIG. 7 shows the expression of various Treg markers in transduced and untransduced fractions of cells and in mock transduced cells.

FIG. 8 shows the % suppression of Tregs against the proliferation of T-effector cells using different stimuli of aCD3/CD28 beads, HLA-A2− B cells and HLA-A2+ B cells.

FIG. 9 shows the % of dead target cells indicating the cytotoxicity of Tregs transduced with different constructs towards target cells.

FIG. 10 shows the level of various intracellular cytokines in mock Tregs and Tregs transduced with the 658 or 786 construct (both transduced and untransduced cell fractions).

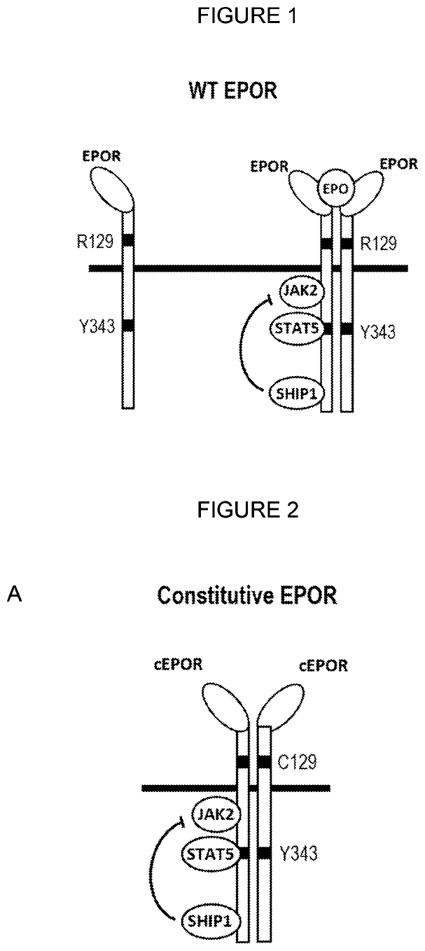

### DETAILED DESCRIPTION

The subject of the products, methods and uses herein is a recombinant protein which can be used to promote the functionality or survival or indeed any property of a cell by which it is expressed. The protein thus has utility in adoptive cell transfer, to assist in the preparation of cells for ACT and/or to help keep the cells alive and functional following transfer to a subject. Therapeutic efficacy of the cell may be improved.

The recombinant protein described herein is based upon the presence of an exodomain, at least a portion of which is derived from the extracellular region of EPOR, wherein the exodomain comprises a dimerisation domain (e.g. at least one dimerisation domain) allowing dimerisation of the recombinant protein with another protein (a second protein) and the provision of a signal to a cell comprising the recombinant protein in the absence of a signal inducer molecule, particularly EPO. The recombinant protein as described herein is capable of spontaneously dimerising with a second protein (e.g. another recombinant protein) and of providing a constitutive signal to a cell in which it is expressed. Particularly, the signal may be transduced to the cell through an endodomain which in a particular embodiment may be comprised within the recombinant molecule or in an alternative embodiment may be provided on a separate protein (a “signalling protein”).

The present invention is thus based on the ability of the recombinant molecule to provide a constitutive signal to the cell. In particular, the dimerisation of the recombinant protein provided herein activates a signalling pathway mediated by JAK kinase activity, including notably JAK1 or JAK2 activity, and especially the JAK1-STAT or JAK2-STAT signalling pathway. In this way the recombinant protein may mimic the signalling which is induced by activation of a natural cytokine receptor, for example an EPOR or an interleukin (e.g. IL-2) receptor. By “mimic”, it is meant that the signaling cascade activated by the recombinant protein of the present disclosure is similar to the signaling cascade activated by a natural cytokine receptor, while the magnitude of activation induced by the recombinant proteins of the present disclosure could be different from that of a natural cytokine receptor.

As described herein, the recombinant protein comprises an exodomain. An “exodomain” as used herein refers to a portion or part of the recombinant protein which when expressed as a membrane bound protein can be found on the outside of the cell. Thus, typically, the exodomain refers to the part of the protein which is found extracellularly and not within the cell membrane or cytoplasm. It will be understood by a skilled person that expression of the recombinant protein will initially occur within the cell and that during this process the whole of the protein will be found within the cell. Further, it is possible that the recombinant protein may be internalised and cycled periodically (e.g. in response to binding of a ligand, such as EPO). However, in its usual form after expression, the recombinant protein typically is a membrane bound protein and the exodomain as part of this protein may be found outside of the cell membrane and the cell. The terms “exodomain” and “extracellular domain” and “extracellular region” are used interchangeably herein.

The exodomain of the recombinant protein comprises a sequence which is derived from the extracellular region of EPOR. For example, the exodomain of the recombinant protein may consist of a sequence which is derived from the extracellular region of EPOR. “Derived from” as used herein means that the exodomain of the recombinant protein comprises a sequence which has at least 70% identity to the extracellular region of EPOR, more particularly, a sequence which has at least 80, 90, 95, 96, 97, 98 or 99% to the extracellular region of EPOR. The sequence derived from the extracellular region of EPOR which is comprised within the exodomain of the recombinant protein, may comprise at least one modification (e.g. at least one amino acid substitution, insertion, deletion or translocation) relative to the wildtype extracellular region of EPOR. In this respect, in one embodiment, the exodomain of the recombinant protein may not comprise the wildtype extracellular region of EPOR, e.g., that shown in SEQ ID NO. 3 or SEQ ID NO. 5.

Therefore, the EPOR extracellular region sequence comprised within the exodomain may comprise one modification, or more than one modification, for example, at least two, three, four, five, ten, fifteen or twenty modifications. Particularly, at least one of the modifications may result in the introduction of a dimerisation domain and in the ability of the recombinant protein to dimerise, particularly homodimerize, and to provide a signal to a cell expressing the recombinant protein in the absence of any signal inducer molecule (particularly in the absence of EPO). The EPOR extracellular region sequence comprised within the exodomain may additionally or alternatively comprise modifications which do not result in the introduction of a dimerisation domain and thus in the ability of the recombinant protein to dimerise and/or signal in the absence of any signal inducer molecule. However, where the dimerisation domain is provided elsewhere in the exodomain (i.e. not within the portion derived from the extracellular domain of EPOR), the EPOR extracellular region sequence within the exodomain may comprise or consist of wildtype sequence or may comprise one or more modifications which do not result in the introduction of a dimerisation domain.

EPOR is a receptor that binds to erythropoietin (EPO) in its natural conformation where EPO binding triggers dimerisation and the JAK2/STAT5 signalling cascade. Wildtype EPOR therefore provides an inducible rather than a constitutive signal to a cell expressing the receptor. Wildtype human EPOR comprises 508 amino acids, including a signal peptide (SEQ ID NO. 6, from amino acids 1-24 of SEQ ID NO. 1), an extracellular region (SEQ ID NO. 3 (without signal peptide), from amino acid residues 25-250 of SEQ ID NO. 1), a transmembrane domain (SEQ ID NO. 7, from amino acid residues 251-273 of SEQ ID NO. 1) and a cytoplasmic domain (SEQ ID NO. 8, from amino acid residues 274-508 of SEQ ID NO. 1). The human full length wildtype sequence for EPOR is shown in SEQ ID NO. 1.

The “extracellular region of EPOR” as used herein refers to the region of EPOR which is usually found extracellularly in the EPOR, i.e., outside the cell. Particularly, when considering human or murine EPOR, the “extracellular region of EPOR” may refer to the sequence as set forth in SEQ ID Nos 3 or 5 (for human) or as set forth in SEQ ID NO. 11 (for murine). Thus, the extracellular region of EPOR may comprise or lack the signal peptide.

As described previously, the exodomain of the recombinant protein described herein comprises a sequence derived from the extracellular region of EPOR, which may comprise one or more modifications. Particularly, at least one of the modifications may enable the recombinant protein to dimerise to a second protein and to provide a signal to a cell comprising the recombinant protein in the absence of the signal inducing molecule (more particularly, the recombinant protein may have an increased ability to dimerise to a second protein in the absence of a signal inducer molecule (particularly EPO) as compared to the wildtype EPOR in the absence of a signal inducer molecule (particularly EPO) and/or may have an increased ability to signal to a cell comprising the recombinant protein as compared to the wildtype EPOR in the absence of a signal inducer molecule (particularly EPO)). Thus, the recombinant protein may have at least a 10, 20, 30, 40, 50, 60, 70, 80 or 90% increase in ability to dimerise and/or to provide a signal as compared to wildtype EPOR in the absence of a signal inducer molecule (e.g. EPO). Increases in dimerisation and/or signalling can be determined as discussed further below.

Typically, one or more modifications which result in the stated function (signalling in the absence of a signal inducer molecule, i.e. constitutive signalling) may be made to the extracellular region of EPOR in the invention. Such one or more modifications may allow dimerisation between the recombinant protein and second protein based on the cognate dimerisation domains which associate e.g., bind or interact in any way, when in proximity. Thus modification of the extracellular region of EPOR may introduce a dimerisation domain allowing dimerisation in the absence of a signal inducer molecule.

In one particular embodiment, the one or more modifications may allow dimerisation by disulphide bonding of the recombinant protein to a second protein (disulphide-linked dimerisation) and more particularly may allow disulphide-linked homodimerisation. Typically, one or more non-naturally occurring cysteine residues may be inserted or substituted into the extracellular region of EPOR to achieve this effect. In this respect, the sequence comprised within the exodomain derived from the extracellular region of EPOR may have an amino acid modification at position 130 of SEQ ID NO. 3, at position 154 of SEQ ID NO. 5 or at position 129 of SEQ ID NO. 11. More particularly, the modification may be an amino acid substitution of arginine to cysteine, and thus may be referred to as R130C, R154C or R129C in the context of SEQ ID NO. 3, SEQ ID NO. 5 (or SEQ ID NO. 1) or SEQ ID NO. 11 (or SEQ ID NO. 9), respectively. It will be appreciated by a skilled person that this embodiment particularly provides for disulphide bonding between recombinant protein chains, providing homodimerization of the recombinant protein. Thus, the exodomain of the recombinant protein may comprise or consist of an amino acid sequence of SEQ ID NO. 3 having an amino acid substitution at position 130 of R to C, SEQ ID NO. 5 having an amino acid substitution at position 154 of R to C or SEQ ID NO. 11 having an amino acids substitution at position 129 of R to C.

Alternatively viewed, the exodomain may comprise or consist of a sequence of SEQ ID NO. 2, or SEQ ID NO. 4, or SEQ ID NO. 10.

Reference to “dimerisation domain” as used herein refers to a first domain which is capable of a binding to a second domain, wherein the first and second domains maybe the same (homodimerization domains) or different (heterodimerisation domains). The dimerisation domain of the invention may also be termed a constitutive dimerisation domain, as dimerisation occurs to its cognate partner in the absence of a signal inducer molecule (e.g. a dimerisation inducer molecule). As discussed above, the dimerisation domain may be present within any part of the exodomain, e.g. within the portion derived from the extracellular region of EPOR or elsewhere within the exodomain. Thus, the dimerisation domain may be formed by a modification to the extracellular region of EPOR as described above, or may be based on other constitutive dimerisation systems known in the art as discussed below (which may be located within the sequence derived from the extracellular region of EPOR, at the N or C terminal ends of the extracellular region of EPOR or elsewhere in the exodomain).

Particular mention may be made in this regard of leucine zippers which are widely known and described in the art. Leucine zipper domains are a type of protein-protein interaction domain commonly found in transcription factors characterized by leucine residues evenly spaced through a a-helix. Thus, in an embodiment a dimerization domain herein is or comprises a leucine zipper sequence. These may be used for hetero- or homodimerization, according to the leucine zipper sequence which is used. Leucine zipper domains derived from Fos or Jun protein molecules are described in Patel et al., 1996, J. Biol. Chem. 271(8), 30386-30391; and Stuhlmann-Laeisz et al., 2006, Mol. Biol. Cell 17, 2986-2995. A representative leucine zipper sequence based on human c-Jun is shown in SEQ ID NO. 20 (this can include GG at N terminus) and a Fos leucine zipper sequence is shown in SEQ ID NO. 21.

Heterodimerization domains comprising Jun and Fos leucine zippers respectively may be used. Alternatively, homodimerization domains comprising Jun leucine zippers may be used.

Other leucine zipper dimerization domains known in the art include those based on ZIP proteins, a class of transcription factors. A ZIP domain is a region of alpha-helix containing leucines which line up to form the leucine zipper motif. A ZIP domain can interact with leucines on other ZIP domains to reversibly hold the alpha-helices together (i.e. to dimerize them). Thus, a dimerization domain herein can comprise a bZIP or aZIP leucine zipper domain. For example, a heterodimerization domain can be or comprise a BZIP(RR) domain which heterodimerizes with an AZIP(EE) domain. Leucine zippers are an example of a coiled coil structural protein motif which may be used to create dimerization domains. Heterodimerization domains based on bZIP and synthetic coiled coil peptides are described in Reinke et al. 2010, J. Am. Chem. Soc. 132(17), 6025-6031 and any of these could be used. For example, suitable leucine zipper domains can include SYNZIP 1 to SYNZIP 48. Other examples of leucine zipper domains include BATF, ATF4, ATF3, BACH1, JUND, NFE2L3, and HEPTAD. The sequence of a BZip (RR) leucine zipper domain is shown in SEQ ID NO. 22. The sequence of a AZip (EE) leucine zipper domain is shown in SEQ ID NO. 23.

In some embodiments, a suitable pair of leucine zipper domains has a dissociation constant (Kd) of 1000 nM or less, for example 100 nM or less, 10 nM or less, or 1 nM or less.

Further exemplary pairs of dimerization domains can include PSD95-Dlgl-zo-1 (PDZ) domains, or a streptavidin domain and a streptavidin binding protein (SBP) domain. Other dimerization domains may be obtained or derived from other proteins known to interact or bind to each other. For example, a heterodimerization domain pair can comprise CD80 and PDL-1.

A still further example of a homodimerization domain is the Fc region of immunoglobulin G. Fc regions have widely been used in fusion proteins, including to provided dimerization domains, and various fragments and mutants of dimerisable Fc regions have been described in the literature, for example a fragment lacking the first 5 amino acids of the Fc region.

As discussed above, the extracellular region sequence from EPOR comprised within the exodomain may comprise additional modifications which may or may not contribute to the ability of the recombinant molecule to dimerise and signal in the absence of a signal inducer molecule. Particularly, it is envisaged by the inventors, that variants or fragments of the extracellular region of EPOR may be utilised in the present invention, as long as the recombinant protein has the desired ability to spontaneously dimerise (i.e. to dimerise in the absence of signal inducer molecules when located with a second protein capable of dimerisation) and to provide a signal to a cell comprising the recombinant protein (and second protein where this is different to the recombinant protein). As indicated above, particularly, such variants or fragments/portions may have at least 70% sequence identity to the wildtype extracellular region of EPOR (e.g. to SEQ ID Nos 3, 5 or 11). Thus, further modified extracellular regions of EPOR may be used in the present invention, e.g. comprising additional amino acid substitutions, deletions, additions or translocations, e.g., at least 1, 2, 3, 4 or 5 amino acid substitutions, deletions, additions or translocations. In this respect, the exodomain may comprise or consist of a variant or fragment of SEQ ID NO. 2, SEQ ID NO. 4 or SEQ ID NO. 10, (e.g. a variant or fragment having at least 60, 70, 80, 90 or 95% identity to SEQ ID NO. 2, SEQ ID NO. 4 or SEQ ID NO. 10), with the proviso that the recombinant protein has the desirable function of dimerising with a second protein and providing a signal to a cell expressing the recombinant protein in the absence of a signal inducer molecule. It will be appreciated that recombinant proteins comprising such variants or fragments may have the same or similar ability to dimerise to a second protein and/or to provide a signal as a recombinant protein comprising an exodomain comprising SEQ ID NO. 2, 4 or 10 or may have a modified ability to dimerise to a second protein. Particularly, a recombinant protein comprising said variant or fragment may have at least 40, 50, 60, 70, 80, 90, 95, 120, 150, 170, 190 or 200% of the ability to dimerise and/or signal as a recombinant protein comprising an exodomain comprising SEQ ID NO. 2. Dimerisation can be detected and measured as described further below.

More particularly, the exodomain may comprise or consist of a variant or fragment of SEQ ID NO 2, SEQ ID NO. 4 or SEQ ID NO. 10 (e.g. a variant or fragment having at least 60, 70, 80, 90 or 95% identity to SEQ ID NO. 2, SEQ ID NO. 4 or SEQ ID NO. 10), with the proviso that the variant or fragment of SEQ ID NO. 2 comprises R130C, the variant or fragment of SEQ ID NO. 4 comprises R154C and the variant or fragment of SEQ ID NO. 10 comprises R129C. Further, said variants or fragments should provide the recombinant protein with the ability to dimerise with a second protein and provide a signal to a cell comprising the recombinant protein in the absence of a signal inducing molecule.

Modifications to the extracellular region of EPOR reported in the art include T114A, S115A, S116A, F117A, F117L, F117W, F117Y, V118A, L120A, E121A, R165A, M174A, S176A, H177A, and R179A. Any one or more of these modifications may be present with exodomain sequence derived from the extracellular region of EPOR in the invention.

Although preferred in some embodiments, it is not essential that the sequence derived from the extracellular region of EPOR comprised with the exodomain of the recombinant protein retains its ability to bind to EPO. Thus, modification to the EPO binding site of the extracellular region of EPOR to reduce (e.g. by more than 10, 20, 30, 40, 50, 60, 70, 80 or 90%) or remove the ability to bind EPO is within the scope of the invention. Alternatively, as discussed, in one embodiment, it may be desirable for the exodomain (and thus the extracellular region of EPOR) to be capable of binding EPO to provide an additional signal to the cell above the constitutive signal provided in view of the modification made to the region. The invention further encompasses the provision of extracellular regions of EPOR wherein the EPO binding site has been specifically modified to provide enhanced binding to EPO, as compared to an unmodified wildtype EPOR extracellular region. In this respect, modification of the EPO binding site may allow an increase of at least 10, 20, 30, 40, 50, 60, 70, 80 or 90% in the affinity of EPO for the binding site. The EPO binding site is found at position 117 of SEQ ID NO. 1 or SEQ ID NO. 5 and thus modification of this position is encompassed by the invention.

The exodomain may further comprise other heterologous domains or tags in addition to the sequence derived from the extracellular region of EPOR and/or in addition to any constitutive dimerisation domains. Thus, reference to an exodomain, at least a portion of which is derived from the extracellular region of EPOR, as used herein, means that although the exodomain comprises an amino acid sequence derived from the extracellular region of EPOR, additional heterologous sequence may be present (i.e. sequence not present in wildtype EPOR). For example, the exodomain may further comprise a suicide moiety to allow the induction of cell death which may be necessary or desirable, for example, during the occurrence of an adverse event during or after cell therapy administration. Examples of possible suicide moieties include CD20 epitopes, where cell death can be induced by the administration of Rituximab. WO2013/15339 describes CD20 epitopes which could be used in this way. Other domains that may be used include tags, which could be used to identify and sort cells expressing the recombinant molecule, for example, Strep tags, or myc tags.

Additionally, it may be desirable in some instances to include one or more further inducible dimerisation domains within the exodomain of the recombinant molecule, to allow possible enhancement of signalling (e.g. an increase in signalling of at least 10, 20, 30, 40 or 50% as compared to a recombinant molecule having the same amino acid sequence but without one or more further inducible dimerisation domain(s)). Alternatively viewed, the presence of one or more further inducible dimerisation domains may allow a greater amount of dimerisation between the recombinant protein as defined herein with a second protein, as compared to the recombinant protein without further inducible dimerisation domains (e.g. an increase of at least 10, 20, 30, 40 or 50%). Any additional inducible dimerisation domains may allow dimerisation by any means, e.g. the dimerisation can be a direct or indirect association and does not mean that the two dimerisation domains need to be bound or be linked directly together, although this is not excluded. Typically, inducible dimerisation domains may each bind to a dimerisation inducer molecule. A skilled person will appreciate that when a dimerisation inducer molecule is required for dimerisation between any further inducible dimerisation domains, the increase in signalling or in the proportion of dimers obtained may only occur in the presence of a dimerisation inducer molecule. Such further inducible dimerisation domains may be in addition to the presence of an EPO binding site within the sequence derived from the extracellular region of EPOR or may be to replace the loss of such an EPO binding site.

Chemically-induced dimerization systems are known in the art, using various dimerisation inducer molecules, and different protein domains for dimerization, and are described further below.

The concept of chemically induced dimerization mediated by small molecule inducers has been known for many years, and has been used as a tool to control dimerization between proteins of interest that are fused to inducer-binding domains. Such systems have been described for use in cell biology for different applications, to bring proteins into proximity, for example to investigate signalling pathways and other biological mechanisms, in medicine to degrade or inactivate pathogenic proteins, and in gene and cell therapy. A typical chemical inducer of dimerization (CID), or dimerization inducer to use the terminology herein, has the feature of being able to interact with, or bind to, two proteins or protein domains, one on either side of the molecule. It thus has two binding sites, or binding surfaces (or more generally, interaction sites). In the case of heterodimerization, a dimerisation inducer is capable of interacting with, or binding to, two different proteins or dimerization domains. In the case of homodimerization, a dimerisation inducer is capable of interacting with, or binding to, two copies, or molecules of the same dimerization domain. The original systems were based on the macrolides FK506 and rapamycin, which are capable of binding to, and therefore inducing heterodimerization of, various different proteins or protein domains, including FK506-binding protein (FKBP), the FKBP-rapamycin domain of mTOR (FRB), calcineurin, and cyclophilin, which can be used in different combinations to achieve heterodimerization domain pairs and CID combinations. Such systems may include the use of cyclosporine, which binds to calcineurin or to cyclophilin, Subsequently, other CID heterodimerization systems based on different molecules have been developed and are described in the literature. Furthermore, homodimerization systems based on FK506 derivatives which are able to bind two FKBP molecules have been developed, i.e. based on symmetric or dimeric inducers, which comprise two binding sites for the same dimerization domain.

In an embodiment the dimerisation inducer molecule is rapamycin or an analogue thereof, and the dimerization domains are protein domains which bind thereto. Rapamycin and rapamycin analogues induce heterodimerization by generating an interface between the FRB domain of mTOR and a FK506-binding protein (FKBP). This association results in FKBP blocking access to the mTOR active site inhibiting its function. While mTOR is a very large protein, the precise small segment of mTOR required for interaction with Rapamycin is known and can be used.

The macrolides rapamycin and FK506 act by inducing the heterodimerization of cellular proteins. Each drug binds with a high affinity to the FKBP12 protein, creating a drug-protein complex that subsequently binds and inactivates mTOR/FRAP and calcineurin, respectively. The FKBP-rapamycin binding (FRB) domain of mTOR has been defined and applied as an isolated 89 amino acid protein moiety that can be fused to a protein of interest. Rapamycin can then induce the approximation of FRB fusions to FKBP12 or proteins fused with FKBP12.

The terms “FRB” and “FKBP” include variants thereof. Such variants may include amino acid sequences having one or more amino acid modifications (e.g. substitutions, additions and/or deletions) relative to the native sequence. The term “FKBP” includes FKBP12.

Rapamycin has several properties of an ideal inducible dimerizer: it has a high affinity (KD<1 nM) for FRB when bound to FKBP, and is highly specific for the FRB domain of mTOR. Rapamycin is an effective therapeutic immunosuppressant with a favourable pharmacokinetic and pharmacodynamics profile in mammals. Pharmacological analogues of Rapamycin with different pharmacokinetic and dynamic properties such as Everolimus, Temsirolimus and Deforolimus (Benjamin et al, Nature Reviews, Drug Discovery, 2011) may also be used according to the clinical setting.

In order to prevent rapamycin binding and inactivating endogenous mTOR, the surface of rapamycin which contacts FRB may be modified. Compensatory mutation of the FRB domain to form a surface that accommodates the “bumped” rapamycin restores dimerizing interactions only with the FRB mutant and not to the endogenous mTOR protein.

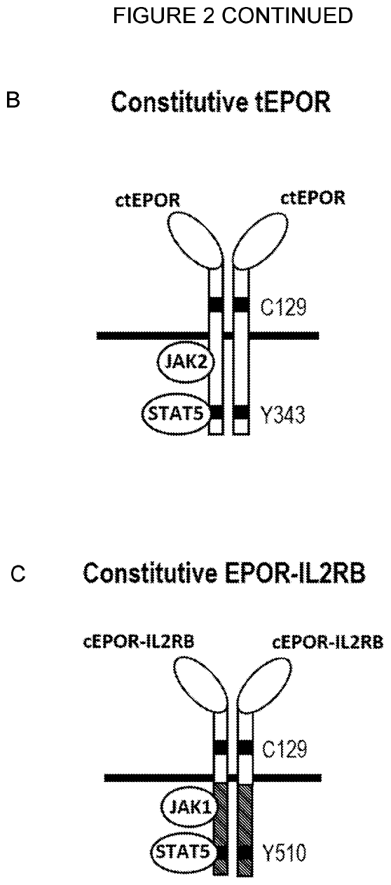

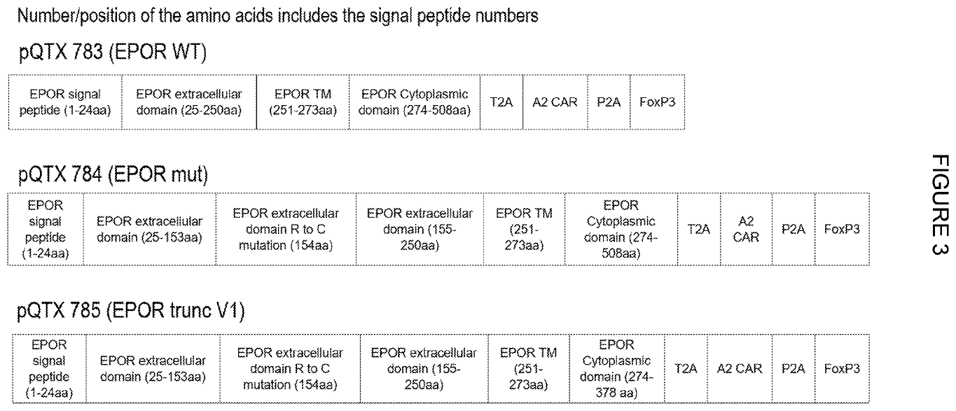

Bayle et al. (Chem Bio; 2006; 13; 99-107) describes various rapamycin analogues, or “rapalogs” and their corresponding modified FRB binding domains. For example, Bayle et al. (2006) describes the rapalogs: C-20-methyllyrlrapamycin (MaRap), C16(S)-Butylsulfonamidorapamycin (C16-BS-Rap) and C16-(S)-7-methylindolerapamycin (AP21976/C16-AiRap), as shown in FIG. 2, in combination with the respective complementary binding domains for each. Other rapamycins/rapalogs include sirolimus and tacrolimus (FK506).

Thus, in such an embodiment, an inducible dimerization domain within a recombinant protein as described herein may comprise FKBP and the second protein may comprise the cognate dimerization domain FRB, or vice versa. FKBP/FRB may have or may comprise a sequence as shown in any one of SEQ ID NO: 15 to SEQ ID NO: 19, or a variant thereof.

The “signal inducer molecule” or “signal inducing molecule” as used interchangeably herein refers to a molecule which is capable of inducing signalling through a protein or receptor comprising an exodomain, transmembrane domain and an endodomain to a cell comprising such a protein or receptor. A signal inducer molecule may be capable of inducing any kind of signal, and a skilled person will appreciate that the signal induced will depend on the endodomain present within a protein or receptor. Typically, the signal inducer molecule binds to the protein or receptor to induce the signal. In one aspect, the signal inducer may bind to the protein or receptor and induce dimerisation or multimerization, resulting in transduction of a signal through the endodomain of the protein or receptor. In this aspect, the signal inducer molecule may be a dimerisation inducer molecule. However, it is also possible for the signal inducer to bind to a receptor or protein and to induce a conformational change resulting in the production of a signal translocated by the endodomain of the protein or receptor. In this respect, a signal inducer molecule may bind to an already dimerised receptor or protein (e.g. non-signalling or weak signalling) to produce a signal in a cell through the endodomain. In the present invention, the recombinant protein is capable of providing a signal to a cell in the absence of such a signal inducer molecule. Thus, the recombinant protein does not need the presence of a signal inducer molecule to provide a signal to a cell (and/or to dimerise with a second protein). Particularly, the signal inducer molecule may be EPO.

“EPO” as used herein refers to erythropoietin and comprises amino acid sequence of SEQ ID NO. 14. EPO typically binds to EPOR at amino acid position 117 of SEQ ID NO. 1. As discussed in detail, the recombinant protein of the invention does not require the presence of EPO to produce a signal to a cell comprising the recombinant protein.

A “dimerisation inducer molecule” as used herein may create an interface between two inducible dimerisation domains (e.g. within the recombinant protein and the second protein) to bring them together as a dimer or that allows chemical cross-linking between two inducible dimerisation domains (e.g. within the recombinant protein and the second protein) allowing the formation of a dimer. As discussed extensively herein, the recombinant protein is capable of dimerisation with a second protein and of providing a signal in a cell comprising the recombinant protein in the absence of a signal inducer molecule, including in the absence of a dimerisation inducer molecule. However, it is not excluded that one or more other inducible dimerisation domains maybe present within the recombinant molecule. Examples of a dimerisation inducer molecule, include rapamycin as discussed above.

Dimerisation of a recombinant protein as described herein to a second protein (either by the modification as discussed herein and/or by the presence of one or more inducible dimerisation domains) can be determined or measured by non-denaturing SDS PAGE or by dynamic light scattering techniques, which are well known in the art. Thus, dimerisation can generally be detected due to the difference in size of the monomeric and the dimeric form of a protein (and in the present invention of the recombinant protein). The ability of a recombinant protein to dimerise with a second protein (particularly to homodimerize) can therefore be measured (or determined) by detecting the presence of dimers of recombinant protein within a cell or cell population. The recombinant protein as described herein, has the ability to dimerise to a second protein in the absence of a signal inducer molecule, particularly EPO. Thus, this ability to dimerise can be determined by the detection of dimeric forms of the recombinant protein when expressed within a cell (either alone for homodimerization or together with a different second protein for heterodimerisation). Typically, the recombinant protein of the invention may dimerise in the absence of a signal inducer molecule to the same or similar extent as the wildtype EPOR dimerises in the presence of EPO. Alternatively, the recombinant protein may have a modified ability to dimerise to a second protein as compared to the wildtype EPOR in the presence of EPO. A modified ability to dimerise may therefore refer to an increase or decrease in the relative amount of recombinant protein in dimeric or monomeric form in a cell or cell population (e.g. an increase or decrease of at least 40, 50, 60, 70, 80, 90, 95% of recombinant protein in dimeric form), as compared to a reference, e.g. as compared to the wildtype EPOR in the presence of EPO or for a variant or fragment of SEQ ID NO. 2 as compared to SEQ ID NO. 2. A skilled person will appreciate that a reduction in dimerisation/signalling of the recombinant protein in the absence of EPO as compared to the wildtype EPOR in the presence of EPO (e.g. a reduction of at least 10, 20, 30, 40 or 50%) will still convey sufficient signal to a cell to provide the desired outcome (e.g. persistence) and may in particular embodiments be preferred.

Dimerisation to a “second protein” as referred to herein, means that the recombinant protein is capable of forming dimers with a second protein (i.e., a recombinant protein monomer can associate with a monomer of a second protein to form a dimer (comprising one copy of each monomer)). The second protein in one aspect is the recombinant protein and thus in this instance, the recombinant protein is capable of homodimerization, e.g., of dimerising to itself, where a dimer comprises of two recombinant protein monomers. Thus, in this embodiment, expression of the recombinant protein in a cell is sufficient for dimerisation to occur (the expression of a different protein is unnecessary for dimerisation), and as discussed previously, no signal inducer molecule is required. Dimerisation can be detected as described above.

Alternatively, or additionally, the recombinant protein as defined herein may be capable of heterodimerisation with a different second protein which may not be the recombinant protein of the invention. The second protein may thus comprise different domain or portions to the recombinant protein. However, the second protein should be capable of dimerising to the recombinant protein and of providing a signal in the absence of a signal inducer molecule. Therefore, the second protein should comprise a cognate dimerisation domain to that of the recombinant protein which are capable of association and of signalling in the absence of a signal inducer molecule. In one embodiment, for example, where the recombinant protein comprises an exodomain comprising a modified extracellular region of EPOR where the modification comprises the insertion or substitution of an amino acid residue to a cysteine (for the purposes of disulphide linked dimerisation), the second protein should comprise a cognate cysteine residue at an appropriate position to allow for dimerisation in the absence of a signal inducer molecule. Alternatively (or additionally), where the recombinant protein comprises an exodomain having a leucine zipper, the second protein may also comprise a cognate leucine zipper to allow for dimerisation in the absence of a signal inducer molecule. For example, the recombinant protein may comprise a Jun or Fos leucine zipper and the second protein may comprise a Jun leucine zipper (or vice versa). A skilled person will appreciate that a recombinant protein as described herein may be capable of homodimerization and heterodimerisation. Thus, a cell expressing the recombinant protein and a second different protein may comprise homodimers of the recombinant protein and also heterodimers of the recombinant protein and the second different protein.

Thus, in one embodiment, the second protein may be a variant or portion of the recombinant molecule, particularly, the second protein may comprise an exodomain which is a variant or portion of the exodomain of the recombinant molecule (e.g. may have at least 70, 80, 90 or 95% sequence identity thereto). Alternatively, the second protein may comprise an endodomain which is a variant or portion of the endodomain of the recombinant molecule (e.g. may have at least 70, 80, 90 or 95% identity thereto). The second protein must be capable of binding to the recombinant protein in the absence of a signal inducer molecule. Particularly, for recombinant proteins comprising additional inducible dimerisation domains, for example inducible heterodimerisation domains, the additional inducible dimerisation domain present within the recombinant protein may be different to the additional inducible dimerisation domain present in the second protein (e.g. FRB/FKBP or functional variants thereof—in this embodiment, the recombinant protein may additionally comprise FRB and the second protein may comprise FKBP and vice versa).

Additionally, or alternatively, the recombinant protein and the second protein may comprise different tags, e.g. the recombinant protein may comprise a strep tag and the second protein may comprise a myc tag or vice versa. It will be appreciated by a skilled person that the second protein may typically be recombinant and that the invention may additionally require expression of said recombinant second protein in a cell together with the recombinant protein of the invention. Thus, the invention additionally provides for introduction of a second nucleic acid molecule comprising a nucleotide sequence encoding a second protein into a cell, e.g. together with a nucleic acid comprising a nucleotide sequence encoding a recombinant protein as defined herein.

Variants of any amino acid sequence presented herein may have at least 80%, 85%, 90%, 95%, 98% or 99% sequence identity to the reference sequence (i.e. to a reference SEQ ID NO. as specified herein), unless stated otherwise. In particular, such a variant retains the desired or required property of the parent molecule from which it is derived, i.e. the reference sequence. Thus, the variant sequence may have the stated % sequence identity provided that the variant sequence provides an effective dimerization system and signal.

The term “derivative” or “variant” as used interchangeably herein, in relation to the present proteins or polypeptides includes any substitution of, variation of, modification of, replacement of, deletion of and/or addition of one (or more) amino acid residues from or to the sequence providing that the resultant protein or polypeptide retains the desired function. For example, where the derivative or variant is an endodomain, the desired function may be the ability of that domain to signal (e.g. activate or inactivate a downstream molecule), where the derivative or variant is a dimerization domain, the desired function is interaction with a cognate dimerisation domain (directly or indirectly). Alternatively viewed, the variants or derivatives referred to herein are typically functional variants or derivatives. For example, variant or derivative may have at least at least 10%, at least 20%, at least 30%, at least 40%, at least 50%, at least 60%, at least 70%, at least 80%, or at least 90% function compared to the corresponding, reference sequence. The variant or derivative may have a similar or the same level of function as compared to the corresponding, reference sequence or may have an increased level of function (e.g. increased by at least 10%, at least 20%, at least 30%, at least 40% or at least 50%).

Typically, amino acid substitutions may be made, for example from 1, 2 or 3 to 10 or 20 substitutions provided that the modified sequence retains the required activity or ability. Amino acid substitutions may include the use of non-naturally occurring analogues. For example, the variant or derivative may have at least 10%, at least 20%, at least 30%, at least 40%, at least 50%, at least 60%, at least 70%, at least 80%, or at least 90% activity or ability compared to the corresponding, reference sequence. The variant or derivative may have a similar or the same level of activity or ability as compared to the corresponding, reference sequence or may have an increased level of activity or ability (e.g. increased by at least 10%, at least 20%, at least 30%, at least 40% or at least 50%).

Proteins or peptides may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to Table 1 below.

TABLE 1

| | | |
|---|---|---|
| ALIPHATIC | Non-polar | G A P |
| | Polar - uncharged | I L V |
| | Polar - charged | C S T M |
| | | N Q |
| | | D E |
| | | K R |
| AROMATIC | | H F W Y |

The derivative may be a homologue. The term “homologue” as used herein means an entity having a certain homology with the wild type amino acid sequence and the wild type nucleotide sequence. The term “homology” can be equated with “identity”.

A homologous or variant sequence may include an amino acid sequence which may be at least 80%, 85% or 90% identical, preferably at least 95%, 96%, 97%, 98% or 99% identical to the subject sequence. Typically, the variants will comprise the same active sites etc. as the subject amino acid sequence. Although homology can also be considered in terms of similarity (i.e. amino acid residues having similar chemical properties/functions), in the context herein it is preferred to express homology in terms of sequence identity.

Homology comparisons can be conducted by eye or, more usually, with the aid of readily available sequence comparison programs. These commercially available computer programs can calculate percentage homology or identity between two or more sequences.

Percentage homology or sequence identity may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum percentage homology/sequence identity therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62. Suitably, the percentage identity is determined across the entirety of the reference and/or the query sequence. Once the software has produced an optimal alignment, it is possible to calculate percentage homology, preferably percentage sequence identity. The software typically does this as part of the sequence comparison and generates a numerical result.

“Fragment” typically refers to a selected region of the polypeptide or polynucleotide that is of interest functionally, e.g. is functional or encodes a functional fragment. “Fragment” thus refers to an amino acid or nucleic acid sequence that is a portion (or part) of a full-length polypeptide or polynucleotide.

Such variants, derivatives and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

The recombinant protein as described herein particularly comprises a transmembrane domain which typically anchors the exodomain of the recombinant protein to the cell membrane. The transmembrane domain may be derived from any protein having a transmembrane domain, including any of the type I, type II or type Ill transmembrane proteins.

The transmembrane domain of the chimeric protein may also comprise an artificial hydrophobic sequence. Additional transmembrane domains will be apparent to those of skill in the art. The TM domain may for example be selected from any of those typically used in recombinant transmembrane proteins. Examples of transmembrane (TM) regions which may be used are: 1) The CD28 TM region (Pule et al, Mol Ther, 2005, November; 12(5):933-41; Brentjens et al, CCR, 2007, September 15; 13(18 Pt 1):5426-35; Casucci et al, Blood, 2013, November 14; 122(20):3461-72.); 2) The OX40 TM region (Pule et al, Mol Ther, 2005, November; 12(5):933-41); 3) The 41BB TM region (Brentjens et al, CCR, 2007, September 15; 13(18 Pt 1):5426-35); 4). The CD3 zeta TM region (Pule et al, Mol Ther, 2005, November; 12(5):933-41; Savoldo B, Blood, 2009, June 18; 113(25):6392-402.); 5) The CD8a TM region (Maher et al, Nat Biotechnol, 2002, January; 20(1):70-5.; Imai C, Leukemia, 2004, April; 18(4):676-84; Brentjens et al, CCR, 2007, September 15; 13(18 Pt 1):5426-35; Milone et al, Mol Ther, 2009, August; 17(8):1453-64.). Other transmembrane domains which may be used include those from CD4, CD45, CD9, CD16, CD22, CD33, CD64, CD80, CD86, or CD154. The transmembrane domain may further be derived from IL2RB or EPOR.

By way of example the transmembrane domain may be derived from the CD28 transmembrane domain, and may comprise or consist of the amino acid sequence shown as SEQ ID NO: 34 or a variant which is at least 80% identical to SEQ ID NO: 24. The variant may be at least 80. 85, 90, 95, 97, 98 or 99% identical to SEQ ID NO: 24.

In a particular embodiment, the transmembrane domain may be derived from the EPOR transmembrane domain, and may comprise or consist of the amino acid sequence shown as SEQ ID NO: 7 or a variant which is at least 80% identical to SEQ ID NO: 7. The variant may be at least 80. 85, 90, 95, 97, 98 or 99% identical to SEQ ID NO: 7.

Alternatively, the transmembrane domain may be derived from the IL2RB transmembrane domain, and may comprise or consist of the amino acid sequence shown as SEQ ID NO: 25 or a variant which is at least 80% identical to SEQ ID NO: 25. The variant may be at least 80. 85, 90, 95, 97, 98 or 99% identical to SEQ ID NO: 25.

Alternatively, the recombinant protein may comprise a domain derived from the CD8a transmembrane domain. Thus, the transmembrane domain may comprise or consist of the amino acid sequence shown as SEQ ID NO: 26 which represents amino acids 183 to 203 of human CD8a, or a variant which is at least 80% identical to SEQ ID NO: 26. Suitably, the variant may be at least 85, 90, 95, 97, 98 or 99% identical to SEQ ID NO: 26.

The transmembrane domain may alternatively be derived from the transmembrane domain of a myeloid receptor protein, e.g. from a TREM protein such as TREM1 or TREM2. Thus, the recombinant protein may comprise a transmembrane domain comprising or consisting of the amino acid sequence shown in SEQ ID NO 27 or SEQ ID NO. 28 or a variant which is at least 80% identical to SEQ ID NO 27 or SEQ ID NO 28. Suitably, the variant may be at least 85, 90, 95, 97, 98 or 99% identical to SEQ ID NO 27 or SEQ ID NO 28.

The recombinant protein as described herein is capable of providing a signal to a cell expressing the recombinant protein (either alone in the case of homodimerization or together with a second protein in the case of heterodimerisation). The signal is typically provided to the cell by an endodomain. Thus, in one embodiment, the recombinant protein of the invention further comprises an endodomain. In an alternative embodiment, the endodomain can be provided by a signalling protein which is a separate protein to the recombinant protein. In this aspect, the recombinant protein and the signalling protein together provide the signal to the cell. Thus, the recombinant protein as described herein may provide the signal directly (e.g. through its own endodomain) or indirectly (through the endodomain of a signalling protein) to a cell.

A “signalling protein” as described herein therefore refers to a protein which is capable of associating with a recombinant protein as defined herein and of transducing a signal to the cell. Typically, the signalling protein therefore comprises a transmembrane domain and an endodomain and may be expressed in a cell together with the recombinant protein (and/or the second protein). In this embodiment, it is particularly envisaged that the signalling protein may associate with the recombinant protein through their respective transmembrane domains where such an association can result in transduction of a signal through the endodomain of the signalling protein. In one particular embodiment, the signalling domain may comprise a transmembrane domain from DAP10 or DAP12 as shown in SEQ ID Nos or, or a variant which is at least 80% identical to SEQ ID NO 27 or SEQ ID NO 28. Suitably, the variant may be at least 85, 90, 95, 97, 98 or 99% identical to SEQ ID NO 27 or SEQ ID NO 28. It will be appreciated by a skilled person that when the transmembrane domain of the signalling protein is from DAP10 or DAP10, the transmembrane domain of the recombinant protein may be derived from a myeloid receptor, particularly from a TREM receptor as discussed above.

The “endodomain” as described herein, as discussed above, may be provided within the recombinant protein or within a separate signalling protein. Particularly however, the recombinant protein may comprise an endodomain. The endodomain comprises a tyrosine kinase activating domain comprising at least a JAK1-binding motif and/or a JAK2-binding motif, and a tyrosine effector domain which can be phosphorylated by the JAK1 and/or JAK2 kinase. Phosphorylation of the tyrosine effector domain allows the signalling cascade to be effected, for example to allow other proteins in the signalling cascade to bind to the effector domain and/or become activated to transmit the signal in the cell. In other words, upon phosphorylation, the tyrosine effector domain can recruit a signal transduction factor. As discussed herein, the recombinant protein is capable of providing a signal to a cell comprising said recombinant protein in the absence of a signal inducer molecule and thus alternatively viewed produces a constitutive signal. A constitutive signal means that a cell is constantly receiving a signal from the recombinant protein. The signal may be increased or reduced as compared to the wildtype EPOR but the recombinant protein is capable of providing the signal constantly. The terms “endodomain”, “intracellular domain or region” and “cytoplasmic domain or region” are used interchangeably herein.

The tyrosine kinase activating domain may in some embodiments also include a JAK3-binding motif. In particular, it may include a JAK1- and a JAK3 binding motif.

In an embodiment, for example where the recombinant protein does not include a JAK3 binding motif in the endodomain, the recombinant protein may be used in conjunction with a second protein which comprises an endodomain domain comprising a JAK3-binding motif.

The endodomain may signal through the JAK-STAT signalling pathway, or in other words, the signal may be mediated by activation of the JAK-STAT signalling pathway.

STAT proteins are transcription factors which are recruited to an activated receptor, and accordingly, in particular the tyrosine effector domain may comprise a STAT association motif, that is a binding site for a STAT. The STAT may be STAT1, STAT2, STAT3, STAT4, STAT5 or STAT6 or any combination thereof. STAT association motifs may be obtained or derived from receptors, including cytokine receptors and receptor tyrosine kinases (RTK). The tyrosine effector domain may contain one or more, e.g. two or more, for example, 3, 4, 5 or more STAT association motifs, which may be the same or different.

By way of example, STAT5 is a transcription factor involved in the IL-2 signalling pathway that plays a key role in Treg function, stability and survival by promoting the expression of genes such as FOXP3, IL2RA and BCLXL. In order to be functional and translocate into the nucleus, STAT5 needs to be phosphorylated. IL-2 ligation results in STAT5 phosphorylation by activating the Jak1/Jak2 and Jak3 kinases via specific signalling domains present in the IL-2Rβ and IL-2Rγ chain, respectively. Although JAK1 (or JAK2) can phosphorylate STAT5 without the need of JAK3, STAT5 activity is increased by the transphosphorylation of both JAK1/JAK2 and JAK3, which stabilizes their activity.

“STAT association motif” as used herein refers to an amino acid motif which comprises a tyrosine and, upon phosphorylation of the tyrosine, is capable of binding a STAT polypeptide. Any method known in the art for determining protein:protein interactions may be used to determine whether an association motif is capable of binding to a STAT. For example, co-immunoprecipitation followed by western blot.

The STAT association motif may for example be a STAT5 association motif which is capable, upon phosphorylation, of binding a STAT5 polypeptide (and similarly for the other STAT polypeptides).

In one embodiment, the STAT association motif is a STAT5 association motif.

Suitably, the endodomain may comprise two (e.g. at least two) or more STAT5 association motifs as defined herein. For example, the signalling domain may comprise two, three, four, five or more STAT5 association motifs as defined herein. In an embodiment, the signalling domain may comprise two or three STAT5 association motifs as defined herein.

Suitably, the STAT5 association motif may exist endogenously in a cytoplasmic domain of a transmembrane protein which may be used to provide the endodomain herein.

For example, the STAT5 association motif may be from an interleukin receptor (IL) receptor endodomain or a hormone receptor.

The endodomain may comprise an amino acid sequence selected from any chain of the interleukin receptors where STAT5 is a downstream component, for example, the cytoplasmic domain comprising amino acid numbers 266 to 551 of IL-2 receptor β chain (NCBI REFSEQ: NP_000869.1, SEQ ID NO: 31), amino acid numbers 292 to 521 of IL-9R chain (NCBI REFSEQ: NP_002177.2, SEQ ID NO: 33), amino acid numbers 257 to 825 of IL-4R a chain (NCBI REFSEQ: NPJD00409.1, SEQ ID NO: 34), amino acid numbers 461 to 897 of IL-3RB chain (NCBI REFSEQ: NP_000386.1, SEQ ID NO: 35) and/or amino acid numbers 314 to 502 of IL-17R p chain (NCBI REFSEQ: NP_061195.2, SEQ ID NO: 36) may be used. It will be appreciated by a skilled person that any one or more of these sequences can be used. The entire region of the cytoplasmic domain of an interleukin receptor chain may be used.

The signalling domain may comprise one or more STAT5 association motifs that comprise an amino acid sequence shown as SEQ ID NO: 31-37 or a variant which is at least 80, 85, 90, 95, 96, 97, 98 or 99% identical to SEQ ID NO: 31-37. For example, the variant may be capable of binding STAT5 to at least 10%, at least 20%, at least 30%, at least 40%, at least 50%, at least 60%, at least 70%, at least 80%, or at least 90% of the level of an amino acid sequence shown as one of SEQ ID NO: 31-37. The variant or derivative may be capable of binding STAT5 to a similar or the same level as one of SEQ ID NO: 31-37 or may be capable of binding STAT5 to a greater level than an amino acid sequence shown as one of SEQ ID NO: 31-37 (e.g. increased by at least 10%, at least 20%, at least 30%, at least 40% or at least 50%).

For example, the STAT5 association motif may be from any one or more of IL2Rβ, IL-3Rβ (CSF2RB), IL-9R, IL-17Rβ, erythropoietin receptor (EPOR), thrombopoietin receptor, growth hormone receptor and prolactin receptor. An endodomain may, for example, comprise STAT association motifs from both IL2RB and EPOR.

The STAT5 association motif may comprise the amino acid motif YXXF/L (SEQ ID NO: 38; wherein X is any amino acid.

Suitably, the STAT5 association motif may comprise the amino acid motif YCTF (SEQ ID NO: 39), YFFF (SEQ ID NO: 40), YLSL (SEQ ID NO: 41), or YLSLQ (SEQ ID NO: 42).

The endodomain may comprise one or more STAT5 association motifs comprising the amino acid motif YCTF (SEQ ID NO: 39), YFFF (SEQ ID NO: 40), YLSL (SEQ ID NO: 41), and/or YLSLQ (SEQ ID NO: 42).

The endodomain may comprise a first STAT5 association motif comprising the amino acid motif YLSLQ (SEQ ID NO: 42) and a second STAT5 association motif comprising the amino acid motif YCTF (SEQ ID NO: 39) or YFFF (SEQ ID NO: 40).

The endodomain may comprise the following STAT5 association motifs: YLSLQ (SEQ ID NO: 42), YCTF (SEQ ID NO: 39) and YFFF (SEQ ID NO: 40).

Association motifs for other STAT polypeptides are known in the art, and may be used. For example, to provide a STAT3 signal to a cell (particularly a Tcon cell), the tyrosine effector domain of the endodomain may comprise YXXQ (SEQ ID NO. 57), where X is any amino acid, for example YRHQ (SEQ ID NO. 58). The STAT3 association motif is present in signalling proteins for example IL-6R, IL10R and IL21R. In one embodiment, the endodomain as defined herein may comprise the cytoplasmic domain of the IL21R alpha chain, e.g. comprising amino acid numbers 256-538 of the IL-21R alpha chain (NCBI RefSeq: NP_068570.1), or a truncated fragment thereof comprising a box 1 motif (amino acid numbers 266 to 274 of NCBI RefSeq: NP_068570.1) required for association with JAK1 and a STAT association motif comprising tyrosine residue 500 (amino acid number 519 of NCBI RefSeq: NP_000869.1) and flanking 3 residues at the C-terminal side of tyrosine residue 500, i.e. YLRQ (SEQ ID NO. 59), required for STAT1/3 association. Alternatively, STAT1 or STAT4 signalling may be provided in a similar manner. For example, a STAT1 association motif may be found at amino acids 335-365 of IL2Rb (subdomain Aci2), as represented by the following sequence:

(SEQ ID NO. 60)
QLLLQQDKVPEPASLSSNHSLTSCFTNQGYF

“JAK1-binding motif” as used herein refers to a BOX motif which allows for tyrosine kinase JAK1 association. Analogously, “JAK2binding motif” as used herein refers to a BOX motif which allows for tyrosine kinase JAK2 association. Suitable JAK1- and JAK2-binding motifs are described, for example, by Ferrao & Lupardus (Frontiers in Endocrinology; 2017; 8(71); which is incorporated herein by reference).

As noted above, the JAK1 and/or JAK2-binding motif may occur endogenously in a cytoplasmic domain of a transmembrane protein.

For example, the JAK1 and/or JAK2-binding motif may be from Interferon lambda receptor 1 (IFNLR1), Interferon alpha receptor 1 (IFNAR), Interferon gamma receptor 1 (IFNGR1), IL10RA, IL20RA, IL22RA, Interferon gamma receptor 2 (IFNGR2) or IL10RB.

The JAK1-binding motif may comprise or consist of an amino acid motif shown as SEQ ID NO: 43-49 or a variant thereof which is capable of binding JAK1.

(SEQ ID NO: 43)
KVLKCNTPDPSKFFSQLSSEHGGDVQKWLSSPFPSSSFSPGGLAPEISP
LEVLERDK
(SEQ ID NO: 44)
NPWFQRAKMPRALDFSGHTHPVATFQPSRPESVNDLFLCPQKELT
(SEQ ID NO: 45)
GYICLRNSLPKVLNFHNFLAWPFPNLPPLEAMDMVEVIYINR
(SEQ ID NO: 46)
PLKEKSIILPKSLISVVRSATLETKPESKYVSLITSYQPFSL
(SEQ ID NO: 47)
RRRKKLPSVLLFKKPSPFIFISQRPSPETQDTIHPLDEEAFLK
(SEQ ID NO: 48)
YIHVGKEKHPANLILIYGNEFDKRFFVPAEKIVINFITLNISDDS
(SEQ ID NO: 49)
RYVTKPPAPPNSLNVQRVLTFQPLRFIQEHVLIPVFDLSGP

The variant of SEQ ID NO: 43-49 may comprise one, two or three amino acid differences compared to any of SEQ ID NO: 21-27 and retain the ability to bind JAK1.

The variant may be at least 80, 85, 90, 95, 96, 97, 98 or 99% identical to any one of SEQ ID NO: 43-49 and retain the ability to bind JAK1.

In a preferred embodiment, the JAK1-binding domain comprises or consists of SEQ ID NO: 43 or a variant thereof which is capable of binding JAK1.

For example, the variant may be capable of binding JAK1 to at least 10%, at least 20%, at least 30%, at least 40%, at least 50%, at least 60%, at least 70%, at least 80%, or at least 90% of the level of a corresponding, reference sequence. The variant or derivative may be capable of binding JAK1 to a similar or the same level as a corresponding, reference sequence or may be capable of binding JAK1 to a greater level than a corresponding, reference sequence (e.g. increased by at least 10%, at least 20%, at least 30%, at least 40% or at least 50%).

A JAK2-binding motif may comprise or consist of an amino acid motif shown as SEQ ID NO: 50-52 or a variant therefore which is capable of binding JAK2.

(SEQ ID NO: 50)
NYVFFPSLKPSSSIDEYFSEQPLKNLLLSTSEEQIEKCFIIEN
(SEQ ID NO: 51)
YWFHTPPSIPLQIEEYLKDPTQPILEALDKDSSPKDDVWDSVSIISFPE
(SEQ ID NO: 52)
YAFSPRNSLPQHLKEFLGHPHHNTLLFFSFPLSDENDVFDKLSVIAEDS
ES

The variant of SEQ ID NO: 50-52 may comprise one, two or three amino acid differences compared to any of SEQ ID NO: 50-52 and retain the ability to bind JAK2.

For example, the variant may be capable of binding JAK2 to at least 10%, at least 20%, at least 30%, at least 40%, at least 50%, at least 60%, at least 70%, at least 80%, or at least 90% of the level of a corresponding, reference sequence. The variant or derivative may be capable of binding JAK2 to a similar or the same level as a corresponding, reference sequence or may be capable of binding JAK2 to a greater level than a corresponding, reference sequence (e.g. increased by at least 10%, at least 20%, at least 30%, at least 40% or at least 50%).

Any method known in the art for determining protein:protein interactions may be used to determine whether a JAK1- or JAK2-binding motif is capable of binding to a JAK1 or JAK2. For example, co-immunoprecipitation followed by western blot.

Suitably, the endodomain may comprise an IL2Rβ endodomain shown as SEQ ID NO: 31; or a variant which has at least 80% sequence identity to SEQ ID NO: 31.

The variant may be at least 80, 85, 90, 95, 96, 97, 98 or 99% identical to SEQ ID NO: 31.

Suitably, the endodomain may comprise a truncated IL2Rβ endodomain shown as any one of SEQ ID NO: 53 or 54 or a variant of any one of SEQ ID NO: 53 or 54 which has at least 80% sequence identity thereto. SEQ ID NO: 53 represents a IL2RB truncated variant with a Y510 mutation. SEQ ID NO: 54 represents a IL2RB truncated variant with Y510 and Y392 mutations.

The variant may be at least 80, 85, 90, 95, 96, 97, 98 or 99% identical to SEQ ID NO: 53 or 54.

In this embodiment, the invention provides a recombinant protein wherein said recombinant protein comprises (i) an exodomain, at least a portion of which is derived from the extracellular region of EPOR, wherein said portion comprises a modification relative to the extracellular region of EPOR allowing dimerization of said recombinant protein with a second protein and provision of a signal into said T cell in the absence of a signal inducer molecule and (ii) an endodomain comprising a truncated IL2Rβ endodomain of SEQ ID NO. 53 or SEQ ID NO. 54, or a variant thereof having at least 80% sequence identity thereto.

In a particular embodiment, the recombinant protein comprises an endodomain, at least a portion of which is derived from the endodomain (cytoplasmic) region of EPOR (SEQ ID NO. 8). In a particular embodiment, the endodomain may comprise an EPOR endodomain as shown in SEQ ID NO 8; or a variant which has at least 40% sequence identity to SEQ ID NO 8. For example, the variant may be at least 45, 50, 55, 60, 65, 70, 75, or 80% identical to SEQ ID NO. 8.

The modifications to the endodomain may be selected depending on the desired level of STAT5 signal.

The variant may have at least 80% sequence identity to SEQ ID NO. 8. The variant may be at least 80, 85, 90, 95, 96, 97, 98 or 99% identical to SEQ ID NO. 8. Any variant may be a functional variant and thus may retain the ability to provide a signal into a cell in which the recombinant protein is expressed. In particular, in embodiments where the endodomain of the recombinant protein does not comprise any other functional (signalling) endodomains in addition to the variant EPOR endodomain, for example wherein the endodomain of the recombinant protein consists of a variant EPOR endodomain, the variant will be a functional variant and thus will retain the ability to provide a signal into a cell in which the recombinant protein is expressed. The signal may particularly signal through the JAK-STAT signalling pathway, particularly JAK2-STAT5 signalling pathway, and therefore may comprise at least one JAK-binding motif (e.g. JAK2-binding motif) and at least one STAT-association motif (e.g. STAT5-association motif) as described herein. The variant EPOR endodomain may, for example, retain at least the tyrosine (Y) residue at position 368 of SEQ ID NO. 1 (Y95 of SEQ ID NO. 8) and/or at least the tyrosine (Y) residue at position 426 of SEQ ID NO. 1 (Y153 of SEQ ID NO. 8). For example, the variant EPOR endodomain may retain only one or both of the tyrosine (Y) residue at position 368 of SEQ ID NO. 1 (Y95 of SEQ ID NO. 8) and the tyrosine (Y) residue at position 426 of SEQ ID NO. 1 (Y153 of SEQ ID NO. 8).

Suitably, the variant may comprise at least one modification to reduce (e.g. eliminate) the binding of SHP1 to the EPOR endodomain sequence, and thus to reduce the negative effect of SHP1 on JAK2. SHP1 may also be referred to as SHIP1 and is a tyrosine phosphatase protein containing an SH2 domain and having an amino acid sequence as shown in SEQ ID NO. 61. SHP1 is known to associate with the activated EPOR and to negatively regulate signalling induced by phosphorylation by JAK2. SHP1 can associate with Y181 and Y183 of the EPOR endodomain of SEQ ID NO. 8. The present invention therefore encompasses, use of an EPOR endodomain sequence where Y181 and/or Y183 of SEQ ID NO. 8 have been modified to reduce the negative effect of SHP1 (e.g. by at least 10, 20, 30, 40, 50, 60, 70, 80, 90, 95, 96, 97, 98, or 99%, for example by up to 100%). Alternatively viewed, modification of Y181 and/or Y183 of SEQ ID NO. 8 may increase signalling to the cell by at least 10, 20, 30, 40, 50, 60, 70, 80 or 90%. Particularly, Y181 and/or Y183 may be deleted from the EPOR endodomain sequence to be used within an endodomain herein, or may be substituted with a different amino acid. For example, the variant EPOR endodomain may comprise a deletion of a sequence of contiguous amino acids encompassing Y181 and Y183 of SEQ ID NO. 8. The sequence of contiguous amino acids encompassing Y181 and Y183 of SEQ ID NO. 8 may be at least 3, 4, 5, 6, 7, 8, 9, 10 or more amino acids in length. For example, the sequence of contiguous amino acids encompassing Y181 and Y183 of SEQ ID NO. 8 may be at least 55 amino acids in length, for example at least 60, 65, 70, or 75 amino acids in length. For example, the sequence of contiguous amino acids encompassing Y181 and Y183 of SEQ ID NO. 8 may be up to 140, 135, 130, 125, 120, 115, 110, 105, 100, 95, 90, 85 or 80 amino acids in length. For example, the variant EPOR endodomain may comprise or consist of a deletion of amino acids 181 to 235 of SEQ ID NO. 8 (amino acids 454 to 508 of SEQ ID NO. 1). For example, the variant EPOR endodomain may comprise or consist of a deletion of amino acids 106 to 235 of SEQ ID NO. 8 (amino acids 379 to 508 of SEQ ID NO. 1). For example, the variant EPO endodomain may comprise or consist of a deletion of amino acids 161 to 235 of SEQ ID NO. 8 (amino acids 434 to 508 of SEQ ID NO. 1). Alternatively, Y181 and/or Y183 may not be modified, particularly if an increased signal to the cell is not required.

Alternatively, the endodomain may comprise or consist of a truncated EPOR endodomain, shown as SEQ ID NO. 62 or a variant thereof having at least 80% sequence identity thereto. The variant may be at least 80, 85, 90, 95, 96, 97, 98 or 99% identical to SEQ ID NO: 62. Thus, typically, a truncated EPOR endodomain may truncate at least the portion of the endodomain comprising Y181 and Y183 of SEQ ID NO. 8. The variant may, for example, include the same truncation as SEQ ID NO: 62 and therefore differ to SEQ ID NO. 62 only by deletions, substitutions or insertions within the sequence of SEQ ID NO. 62. Any variant may be a functional EPOR endodomain variant as described above.

The endodomain of the recombinant protein may comprise or consist of a truncated EPOR endodomain, shown as SEQ ID NO. 106 or a variant thereof having at least 80% sequence identity thereto. The variant may be at least 80, 85, 90, 95, 96, 97, 98 or 99% identical to SEQ ID NO: 106. The variant may truncate at least the portion of the endodomain comprising Y181 and Y183 of SEQ ID NO. 8. The variant may, for example, include the same truncation as SEQ ID NO: 106 and therefore differ to SEQ ID NO. 106 only by deletions, substitutions or insertions within the sequence of SEQ ID NO. 106. Any variant may be a functional EPOR endodomain variant as described above.

The endodomain of the recombinant protein may comprise or consist of a truncated EPOR endodomain, shown as SEQ ID NO. 107 or a variant thereof having at least 80% sequence identity thereto. The variant may be at least 80, 85, 90, 95, 96, 97, 98 or 99% identical to SEQ ID NO: 107. The variant may truncate at least the portion of the endodomain comprising Y181 and Y183 of SEQ ID NO. 8. The variant may, for example, include the same truncation as SEQ ID NO: 107 and therefore differ to SEQ ID NO. 107 only by deletions, substitutions or insertions within the sequence of SEQ ID NO. 107. Any variant may be a functional EPOR endodomain variant as described above.

The variant EPOR endodomain may, for example, comprise an insertion of one or more amino acids. In particular embodiments, the insertion may be at the C-terminus of the variant EPOR endodomain, which may be at the C-terminus of the recombinant protein if the variant EPOR endodomain is located at the C-terminus of the recombinant protein. However, the insertion may also be elsewhere within the variant EPOR endodomain. For example, the variant EPOR endodomain may comprise an insertion of two, three, four, five, six, seven, eight, nine, ten, or more amino acids. For example, the variant EPOR endodomain may comprise an insertion of from one to thirty amino acids, for example from two to twenty, for example from five to fifteen amino acids. For example, the variant EPOR endodomain may comprise an insertion of at least five, six, seven, eight, nine or ten amino acids, for example at its C-terminus. The at least five amino acids may have (comprise or consist of) the sequence MDTVP (SEQ ID NO. 108), or a sequence differing to SEQ ID NO. 108 by no more than one or no more than two amino acids (e.g. deletion and/or substitution). The at least six amino acids may have the sequence SMDTVP (SEQ ID NO. 109), or a sequence differing to SEQ ID NO. 109 by no more than one or no more than two amino acids (e.g. deletion and/or substitution). The at least seven amino acids may have (comprise or consist of) the sequence ASMDTVP (SEQ ID NO. 110), or a sequence differing to SEQ ID NO. 110 by no more than one or no more than two amino acids (e.g. deletion and/or substitution). The at least eight amino acids may have (comprise or consist of) the sequence LASMDTVP (SEQ ID NO. 111), or a sequence differing to SEQ ID NO. 111 by no more than one or no more than two amino acids (e.g. deletion and/or substitution). The at least nine amino acids may have (comprise or consist of) the sequence ALASMDTVP (SEQ ID NO. 112), or a sequence differing to SEQ ID NO. 112 by no more than one or no more than two amino acids (e.g. deletion and/or substitution). The at least ten amino acids may have (comprise or consist of) the sequence PALASMDTVP (SEQ ID NO. 113), or a sequence differing to SEQ ID NO. 113 by no more than one or no more than two amino acids (e.g. deletion and/or substitution). For example, the variant EPOR endodomain may comprise an insertion of five amino acids (e.g. SEQ ID NO. 108), six amino acids (e.g. SEQ ID NO. 109), seven amino acids (e.g. SEQ ID NO. 110), eight amino acids (e.g. SEQ ID NO. 111), nine amino acids (e.g. SEQ ID NO. 112) or ten amino acids (e.g. SEQ ID NO. 113), for example at its C-terminus. In particular embodiments, the variant EPOR endodomain may comprise an insertion of at least ten, e.g. ten, amino acids, for example wherein the at least ten amino acids have the sequence of SEQ ID NO. 113 or a sequence differing to SEQ ID NO. 113 by no more than one or no more than two amino acids, for example at its C-terminus. The inserted amino acids described may be located at the C-terminus of a wild-type EPOR endodomain (i.e. at the C-terminus of SEQ ID NO. 8) or may be located at the C-terminus of a EPOR endodomain also having other modifications, for example located at the C-terminus of a truncated EPOR endodomain as described herein (e.g. at the C-terminus of SEQ ID NO. 62, 106 or 107). Alternatively, the insertion may be located within an EPOR endodomain that is otherwise a wild type EPOR endodomain (except for the insertion) or within an EPOR endodomain also having other modifications, for example within a truncated EPOR endodomain (e.g. within SEQ ID NO. 62, 106 or 107).

The inserted one or more amino acids at the C-terminus of the variant EPOR endodomain may, for example, increase or stabilise cell surface expression of the recombinant protein and/or may increase sensitivity to EPO and/or may increase activation of the JAK-STAT signalling pathway when expressed in a cell (e.g. a Treg cell), as compared to the same cells expressing the same recombinant protein without the insertion. The increase may be at least 10, 20, 30, 40 or 50%. The inserted one or more amino acids at the C-terminus of the variant EPOR endodomain may therefore be particularly useful in embodiments wherein the portion of the exodomain that is derived from the extracellular domain of EPOR retains its ability to bind EPO, as described in exemplary embodiments herein. Increase in sensitivity to EPO may be determined by measuring activation of JAK-STAT signalling as described elsewhere herein. Recombinant proteins having increased sensitivity to EPO will provide a greater JAK-STAT signal at the same concentration of EPO (particularly at low concentrations of EPO such as equal to or less than about 0.1 U/ml, for example equal to or less than about 0.01 U/ml or equal to or less than about 0.001 U/ml).

The endodomain of the recombinant protein may, for example, comprise or consist of a variant EPOR endodomain shown in SEQ ID NO. 114, SEQ ID NO. 115 or SEQ ID NO. 116 or a variant thereof having at least 80% sequence identity thereto. The variant may be at least 80, 85, 90, 95, 96, 97, 98 or 99% identical to SEQ ID NO: 114, 115 or 116 respectively. The variant may truncate at least the portion of the endodomain comprising Y181 and Y183 of SEQ ID NO. 8. Any variant may be a functional EPOR endodomain variant as described above.

STAT, e.g. STAT5, activity is increased by the transphosphorylation of both a JAK1/2 and JAK3, as this stabilizes their activity. As noted above, the endodomain, or more particularly the tyrosine kinase activating domain thereof, may further comprise a JAK3-binding motif. “JAK3-binding motif” as used herein refers to a BOX motif which allows for tyrosine kinase JAK3. Suitable JAK3-binding motifs are described, for example, by Ferrao & Lupardus (Frontiers in Endocrinology; 2017; 8(71); which is incorporated herein by reference).

Any method known in the art for determining protein:protein interactions may be used to determine whether a motif is capable of binding to JAK3. For example, co-immunoprecipitation followed by western blot.

The JAK3-binding motif may occur endogenously in a cytoplasmic domain of a transmembrane protein.

For example, the JAK3-binding motif may be from an IL-2Rγ polypeptide. A functional truncated or variant IL2Rγ polypeptide may be used within the endodomain, wherein the functional truncated or variant IL2Rγ polypeptide retains JAK3-binding activity (e.g. at least 20, 30, 40, 50, 60, 70, 80, 90 or 95% of binding activity of IL2Rγ). Particularly, a truncated IL2Rγ comprising a JAK3-binding motif and a truncated IL2Rβ comprising a STAT5 association motif, and a JAK1-binding motif may be comprised in the endodomain as defined herein. Functional truncations may provide an advantage of reducing construct size for expression.

The JAK3-binding motif may comprise or consist of an amino acid motif sequence shown as SEQ ID NO: 55 or SEQ ID NO: 56 or a variant thereof which is capable of binding JAK3 (e.g. a functional variant or fragment having at least 80, 85, 90, 95 or 99% identity to SEQ ID Nos 55 or 56).

The variant may be at least 80, 85, 90, 95, 96, 97, 98 or 99% identical to SEQ ID NO: 55 or SEQ ID NO: 56.

In a particular embodiment, the signalling domain comprises one or more JAK1-binding domains and at least one JAK3-binding domain/motif (e.g. at least 2 or 3 JAK3-binding domains/motifs).

It will be appreciated by a skilled person that the polynucleotide sequence encoding the JAK3-binding domain may be positioned upstream or downstream (5′ or 3′) of the polynucleotide sequence encoding the tyrosine effector domain, for example, the STAT, e.g. STAT 5, association motif and JAK1 and/or JAK2 binding motif. Typically, the JAK1 and/or JAK2 binding motif would be upstream (5′) of the tyrosine effector domain, e.g. STAT/STAT5, but this may be varied. Particularly, the polynucleotide encoding the JAK3-binding domain may be positioned downstream (3′) of the polynucleotide encoding the STAT association motif and the JAK1/JAK2, binding motifs. Thus, alternatively viewed, in the endodomain as described herein, the JAK3-binding domain may be N or C terminal to the tyrosine effector domain (e.g. STAT association motif) and the JAK1 and/or JAK2 binding domain, preferably C terminal. In one embodiment, the JAK3-binding domain and the STAT association motif/JAK1/2-binding domains are positioned directly adjacent to one another (i.e. are not separated distally by sequence). In a particular embodiment, the JAK3 binding domain is translated in reverse orientation, thus the JAK3 binding motif may comprise a sequence in the reverse orientation to SEQ ID Nos 55 or 56 (e.g. as shown in SEQ ID NO. 63). The polynucleotide encoding the signalling domain may thus comprise nucleotide sequences in the following order: 5′-3′ JAK1, 5′-3′ STAT association motif, 3′-5′ JAK3.

In a particular embodiment, a linker or a hinge may be present between the JAK3-binding motif and the STAT association motif/JAK1, or JAK2, binding motifs. The linker or hinge may comprise or consist of at least 2, 3, 4, 5, 6, 7, 8, 9, 10, 11, 12, 13, 14, 15, 16, 17, 18, 19, 20, 25 or 30 amino acids, e.g. at least 2, 3, 4, 5, 6, 7, 8, 9 or 10 glycine residues. In a most particular embodiment, the endodomain comprises a first amino acid sequence derived from IL2Rγ comprising a JAK3-binding domain (e.g. SEQ ID Nos 55 or 56) and a second amino acid sequence derived from IL2Rβ comprising a STAT5 association motif and a JAK1 binding motif (e.g. SEQ ID NOs 43 or 44), where the first and second amino acid sequences are connected or joined by a linker or hinge.

The endodomain may provide other signalling functions (e.g. those capable of providing a pro-survival or persistence signal, a signal which maintains cell phenotype or induces activation or function in addition to providing a STAT signal), and thus may comprise further domains which are capable of providing such signalling functions.

The endodomain for example, may additionally comprise an intracellular signalling domain such as ζ chain endodomain of the T-cell receptor or any of its homologs (e.g., η chain, FcεR1γ and β chains, MB1 (Iga) chain, B29 (Igs) chain, etc.), CD3 polypeptide domains (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28. The intracellular signaling domain may comprise human CD3 zeta chain endodomain, FcγRIII, FcsRI, cytoplasmic tails of Fc receptors, immunoreceptor tyrosine-based activation motif (ITAM) bearing cytoplasmic receptors or combinations thereof.

Thus, the endodomain may comprise the intracellular signaling domain of a human CD3 zeta chain, which in one embodiment comprises or consists of the following sequence:

UNIPROT: P20963, CD3Z_HUMAN, position 31-143
(SEQ ID NO: 64)
RVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTAT
KDTYDALHMQALPPR

In one embodiment, the endodomain comprises an intracellular signaling domain comprising an amino acid sequence having at least 85, 90, 95, 97, 98 or 99% identity to SEQ ID NO: 64.

The intracellular signaling domain of the chimeric protein may comprise the following CD28 signaling domain:

(SEQ ID NO: 65)
RSKRSRLLHSDYMNMTPRRPGPTRKHYQPYAPPRDFAAYRS

In one embodiment, the intracellular signaling domain comprises a signaling motif which has at least 85, 90, 95, 97, 98 or 99% identity to SEQ ID NO: 65.

The intracellular signaling domain of the endodomain may comprise the following CD27 signaling domain:

(SEQ ID NO: 66)
QRRKYRSNKGESPVEPAEPCHYSCPREEEGSTIPIQEDYRKPEPACSP.

In one embodiment, the intracellular signaling domain comprises a signaling motif which has at least 85, 90, 95, 97, 98 or 99% identity to SEQ ID NO: 66.

Additional intracellular signaling domains will be apparent to those of skill in the art and may be used in connection with alternate embodiments of the invention.

In this aspect, the endodomain may comprise additional domains or sequences which provide transcription factor activity to the cell in which it is expressed, e.g. a transcription factor which has importance for phenotype or function of the cell. For Tregs for example, the signalling domain may additionally be capable of providing the cell with FOXP3, c-Rel, Runx, Ets-1, CREB, NFAT and/or JunB (directly or indirectly). Particularly, the endodomain may be capable of providing a FOXP3 activating or inducing signal to the cell. In one aspect, the endodomain may comprise FOXP3 (or any functional variant, truncation or isoform thereof), wherein the FOXP3 may be cleavable from the chimeric protein upon induction with a CID (for example, using a Notch system). In this instance any cleavable portion (e.g. FOXP3) would be present at the C-terminus of the endodomain. As noted above the various domains, and individual parts of the domains (e.g. the motifs in the endodomain) may be linked to one another by linkers.

A linker as referred to herein is an amino acid sequence which links one domain or part of the protein to another. The linker sequence may be any amino acid sequence which functions to link, or connect, two domains or parts thereof together, such that they may perform their function. Thus a linker may space apart the elements which are linked.

The nature of the linker, in terms of its amino acid composition and/or sequence of amino acids may be varied and is not limited. However, the linker may be a flexible linker. It may thus comprise or consist of amino acids known to confer a flexible character to the linker (as opposed to a rigid linker).

Flexible linkers are a category of linker sequences well known and described in the art. Linker sequences are generally known as sequences which may be used to link, or join together, proteins or protein domains, to create for example fusion proteins or chimeric proteins, or multifunctional proteins or polypeptides. They can have different characteristics, and for example may be flexible, rigid or cleavable. Protein linkers are reviewed for example in Chen et al., 2013, Advanced Drug Delivery Reviews 65, 1357-1369, which compares the category of flexible linkers with those of rigid and cleavable linkers. Flexible linkers are also described in Klein et al., 2014, Protein Engineering Design and Selection, 27(10), 325-330; van Rosmalen et al., 2017, Biochemistry, 56,6565-6574; and Chichili et al., 2013, Protein Science, 22, 153-167.

A flexible linker is a linker which allows a degree of movement between the domains, or components, which are linked. They are generally composed of small non-polar (e.g. Gly) or polar (e.g. Ser or Thr) amino acid residues. The small size of the amino acids provides flexibility and allows for mobility of the connected parts (domains or components). The incorporation of polar amino acids can maintain the stability of the linker in aqueous environments by forming hydrogen bonds with water molecules. The most commonly used flexible linkers have sequences primarily composed of Ser and Gly residues (so-called “GS linkers”). However, many other flexible linkers have also been described (see Chen et al, 2013, supra, for example), which may contain additional amino acids such as Thr and/or Ala, and/or Lys and/or Glu which may improve solubility. Any flexible linker known and reported in the art may be used.

The use of GS linkers, or more particularly GS (“Gly-Ser”) domains in linkers, may allow the length of the linker readily to be varied by varying the number of GS domain repeats, and so such linkers represent one suitable class of linkers. However, flexible linkers are not limited to those based on “GS” repeats, and other linkers comprising Ser and Gly residues dispersed throughout the linker sequence have been reported, including in Chen et al., supra.

In one embodiment, the linker sequence comprises at least one Gly-Ser domain composed solely of Ser and Gly residues. In such an embodiment, the linker may contain no more than 15 other amino acid residues, e.g. no more than 14, 13, 12, 11, 10, 9, 8, 6, 7, 5, or 4 other amino acid residues.

The Gly-Ser domain may have the formula:

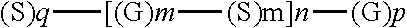

wherein q is 0 or 1; m is an integer from 1-8; n is an integer of at least 1 (e.g. from 1 to 8, or more particularly 1 to 6); and p is 0 or an integer from 1 to 3.

More particularly, the Gly-Ser domain may have the formula:

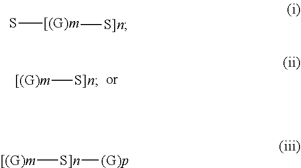

wherein m is an integer from 2-8 (for example 3-4); n is an integer of at least 1 (for example from 1 to 8, or more particularly 1 to 6); and p is 0 or an integer from 1 to 3.

In a representative example, the Gly-Ser domain may have the formula:

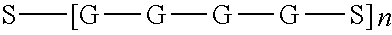

wherein n is an integer of at least one (preferably 1 to 8, or 1-6, 1-5, 1-4, or 1-3). In the formula above, the sequence GGGGS is SEQ ID NO. 70.

However, it is not required for all linkers to be flexible, and in some cases the linker sequence is not a flexible linker sequence. Where the linker connects an interaction domain, or D1/Ht1 or D2/Ht2, to a signalling domain, it is preferably a flexible linker.

Although the length of the linker may not be critical, it may in some cases be desirable to have a shorter linker sequence, or a longer linker sequence, depending on what domains etc. are being linked.

In some cases, the linker may be from any one of 2, 3, 4, 5 or 6 to any one of 24, 23, 22 or 21 amino acids in length. In other cases, it may be from any one of 2, 3, 4, 5 or 6 to any one of 21, 20, 19, 18, 17, 16, or 15 amino acids in length. In other cases, it may be intermediate between these ranges, from example from 6 to 21, 6 to 20, 7 to 20, 8-20, 9-20, 10-20, 8-18, 9-18, 10-18, 9-17, 10-17, 9-16, 10-16 etc. It may accordingly be within a range made up from any of the integers listed above.

In other cases, the linker may be of longer length, for example, from any one of 4, 5, 6, 7, 8, 9, 10, 12, 15, 20 to any one of 100, 90, 80, 70, 60, 50, 45, 40, 30, 28, 25 or 24 amino acids in length. In other cases, it may be intermediate between this range and any of the ranges indicated above. It may accordingly be within a range made up from any of the integers listed above.

The use of GS linkers, or more particularly GS (“Gly-Ser”) domains in linkers, may allow the length of the linker readily to be varied by varying the number of GS domain repeats, and so such linkers represent an advantageous type of linker to use. However, flexible linkers are not limited to those based on “GS” repeats, and other linkers comprising Ser and Gly residues dispersed throughout the linker sequence have been reported, including in Chen et al., supra.

A linker sequence may be composed solely of, or may consist of, one or more Gly-Ser domains as described or defined above. However, as noted above, the linker sequence may comprise one or more Gly-Ser domains, and additional amino acids. The additional amino acids may be at one or both ends of a Gly-Ser domain, or at one or both ends of a stretch of repeating Gly-Ser domains. Thus, the additional amino acid, which may be other amino acids, may lie at one or both ends of the linker sequence, e.g. they may flank the Gly-Ser domain(s). In other embodiments, the additional amino acids may lie between Gly-Ser domains. For example, two Gly-Ser domains may flank a stretch of other amino acids in the linker sequence. Further, as also noted above, in other linkers, GS domains need not be repeated, and G and/or S residues, or a short domain such as GS, may simply be distributed along the length or the sequence.

Representative exemplary linker sequences are listed below:

(SEQ ID NO. 68)
ETSGGGGSRL
(SEQ ID NO. 69)
SGGGGSGGGGSGGGGS
S(GGGGS)₁₋₅ (where GGGGS is SEQ ID NO. 70)
(GGGGS)₁₋₅ (where GGGGS is SEQ ID NO. 70)
(SEQ ID NO. 71)
SGGGGSGGGGS
S(GGGS)₁₋₅ (where GGGS is SEQ ID NO. 67)
(GGGS)₁₋₅ (where GGGS is SEQ ID NO. 67)
(SEQ ID NO. 72)
SGGGGSGGGGSGGGGSGGGGS
(SEQ ID NO. 73)
SGGGGSGGGGSGGGGSGGGGSGGGGS
S(GGGGGS)₁₋₅ (where GGGGGS is SEQ ID NO. 74)
(GGGGGS)₁₋₅ (where GGGGGS is SEQ ID NO. 74)
S(GGGGGGS)₁₋₅ (where GGGGGGS is SEQ ID NO. 75)
(GGGGGGS)₁₋₅ (where GGGGGGS is SEQ ID NO. 75)
(SEQ ID NO. 76)
G6
(SEQ ID NO. 77)
G8
(SEQ ID NO. 78)
KESGSVSSEQLAQFRSLD
(SEQ ID NO. 79)
EGKSSGSGSESKST
(SEQ ID NO. 80)
GSAGSAAGSGEF
(SEQ ID NO. 81)
SGGGGSAGSAAGSGEF
(SEQ ID NO. 82)
SGGGLLLLLLLLGGGS
(SEQ ID NO. 83)
SGGGAAAAAAAAGGGS
(SEQ ID NO. 84)
SGGGAAAAAAAAAAAAAAAAGGGS
(SEQ ID NO. 85)
SGALGGLALAGLLLAGLGLGAAGS
(SEQ ID NO. 86)
SLSLSPGGGGGPAR
(SEQ ID NO. 87)
SLSLSPGGGGGPARSLSLSPGGGGG
(SEQ ID NO. 88)
GSSGSS
(SEQ ID NO. 89)
GSSSSSS
(SEQ ID NO. 90)
GGSSSS
(SEQ ID NO. 91)
GSSSSS
(SEQ ID NO. 92)
SGGGGS.

For linking motifs within a signalling domain, the following linkers can be mentioned:

(SEQ ID NO. 93)
GGGGSGGGGSGGGGS
(SEQ ID NO. 94)
GGGGG
(SEQ ID NO. 95)
GGGGSGGGGS
(SEQ ID NO. 96)
GGGGSGGGGSGGGGSGGGGS
(SEQ ID NO. 97)
GGGGGGG
(SEQ ID NO. 98)
GGGGGGGGG.

As used herein, the terms “polynucleotide” and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed. Nucleotide sequences encoding the various domains and motifs etc. described herein are known and available in the art, and any of these may be used or modified for use herein.

Nucleic acids according to the invention may comprise DNA or RNA. They may be single-stranded or double-stranded. They may also be polynucleotides which include within them synthetic or modified nucleotides. A number of different types of modification are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest. The terms “variant”, “homologue” or “derivative” in relation to a nucleotide sequence include any substitution of, variation of, modification of, replacement of, deletion of or addition of one (or more) nucleic acid from or to the sequence.

Nucleic acid molecules/polynucleotides/nucleotide sequences such as DNA nucleic acid molecules/polynucleotides/sequences may be produced recombinantly, synthetically or by any means available to those of skill in the art. They may also be cloned by standard techniques.

Longer nucleic acid molecules/polynucleotides/nucleotide sequences will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

A nucleic acid construct may comprise the nucleic acid molecule together with one or more other nucleotide sequences, for example, regulatory sequences, e.g. expression control sequences, and/or other coding sequences. In particular, the other coding sequence may encode a protein of interest. This may be a therapeutic protein.

As noted above, a recombinant protein may be co-expressed with another protein of interest, for example a second protein or a receptor, particularly an antigen receptor, for example, a CAR or a TCR or a derivative thereof (e.g. a TCR-CAR construct, or single chain TCR construct etc.). The coding sequence for such a further protein, e.g. receptor, may be comprised within a construct as referred to above.

The recombinant protein may also be co-expressed with a safety switch polypeptide. A safety switch polypeptide provides a cell in or on which it is expressed with a suicide moiety. This is useful as a safety mechanism which allows a cell which has been administered to a subject to be deleted should the need arise, or indeed more generally, according to desire or need, for example once a cell has performed or completed its therapeutic effect. Alternatively, as discussed above, the recombinant protein may comprise a suicide moiety.

A suicide moiety possesses an inducible capacity to lead to cellular death, or more generally to elimination or deletion of a cell. An example of a suicide moiety is a suicide protein, encoded by a suicide gene, which may be expressed in or on a cell alongside a desired transgene, in this case the recombinant protein (and optionally a CAR or other receptor which is co-expressed by the cell along with the present recombinant protein), which when expressed allows the cell to be deleted to turn off expression of the transgene (CAR). A suicide moiety herein is a suicide polypeptide that is a polypeptide that under permissive conditions, namely conditions that are induced or turned on, is able to cause the cell to be deleted.

The suicide moiety may be a polypeptide, or amino acid sequence, which may be activated to perform a cell-deleting activity by an activating agent which is administered to the subject, or which is active to perform a cell-deleting activity in the presence of a substrate which may be administered to a subject. In a particular embodiment, the suicide moiety may represent a target for a separate cell-deleting agent which is administered to the subject. By binding to the suicide moiety, the cell-deleting agent may be targeted to the cell to be deleted. In particular, the suicide moiety may be recognised by an antibody, and binding of the antibody to the safety switch polypeptide, when expressed on the surface of a cell, causes the cell to be eliminated, or deleted.

The suicide moiety may be HSV-TK or iCasp9 as is known in the art. However, in other examples the suicide moiety may be, or may comprise an epitope which is recognised by a cell-deleting antibody or other binding molecule capable of eliciting deletion of the cell.

The term “delete” as used herein in the context of cell deletion is synonymous with “remove” or “ablate” or “eliminate” The term is used to encompass cell killing, or inhibition of cell proliferation, such that the number of cells in the subject may be reduced. 100% complete removal may be desirable but may not necessarily be achieved. Reducing the number of cells, or inhibiting their proliferation, in the subject may be sufficient to have a beneficial effect.

In particular, the suicide moiety may be a CD20 epitope which is recognised by the antibody Rituximab. Thus, in the safety switch polypeptide the suicide moiety may comprise a minimal epitope based on the epitope from CD20 that is recognised by the antibody Rituximab. More particularly, the polypeptide may comprise two CD20 epitopes R1 and R2 that are spaced apart by a linker L.

Safety switches based on Rituximab epitopes are described in WO2013/15339. Peptides which mimic the epitope recognised by Rituximab (so-called mimotopes) have been developed, and these were used in WO2013/15339 as a suicide moiety in a combined suicide-marker polypeptide construct also comprising a CD34 minimal epitope as a marker moiety. Specifically, WO2013/15339 discloses a polypeptide termed RQR8, having the sequence set forth in SEQ ID NO. 99, which comprises two CD20 minimal epitopes, separated from one another by spacer sequences and an intervening CD34 marker sequence, and further linked to a stalk sequence which allows the polypeptide to project from the surface of a cell on which it is expressed. The safety switch polypeptide may be RQR8 or a variant thereof having at least 80% sequence identity thereto, e.g. at least 85, 88, 90, 95, 96, 97, 98, or 99% sequence identity thereto. Other safety switch polypeptides which may be used as the basis of safety switch domains include those described in our co-pending PCT patent application No. PCT/EP2021/064053 (WO 2021/239812).

Other polypeptides which may be co-expressed with the chimeric protein or recombinant protein described herein include transcription factors, growth factors or other factors which may assist in enhancing functionality of survival of the cell. For example, the transcription factor FOXP3 may be used to maintain the suppressive phenotype of Treg cells. “FOXP3” is the abbreviated name of the forkhead box P3 protein. FOXP3 is a member of the FOX protein family of transcription factors and functions as a master regulator of the regulatory pathway in the development and function of regulatory T cells. “FOXP3” as used herein encompasses variants, isoforms, and functional fragments of FOXP3. A “FOXP3 polypeptide” is a polypeptide having FOXP3 activity i.e. a polypeptide able to bind FOXP3 target DNA and function as a transcription factor regulating development and function of Tregs. Expression of FOXP3 together with the recombinant proteins described herein in a Treg may further assist in maintaining Treg phenotype.

Co-expression of FOXP3 with a constitutively active recombinant receptor may assist in increasing FOXP3 expression within the cell and maintaining the suppressive phenotype of Treg cells or cells with a regulatory phenotype.

“Increasing FOXP3 expression” means to increase the levels of FOXP3 mRNA and/or protein in a cell (or population of cells) in comparison to a corresponding cell which has not been modified (or population of cells) by introduction of the nucleic acid molecule or vector. For example, the level of FOXP3 mRNA and/or protein in a cell modified according to the present invention (or a population of such cells) may be increased to at least 1.5-fold, at least 2-fold, at least 5-fold, at least 10-fold, at least 50-fold, at least 100-fold, at least 150-fold greater than the level in a corresponding cell which has not been modified according to the present invention (or population of such cells). Preferably the cell is a Treg or the population of cells is a population of Tregs.

Suitably, the level of FOXP3 mRNA and/or protein in a modified cell (or a population of such cells) may be increased to at least 1.5-fold greater, 2-fold greater, or 5-fold greater than the level in a corresponding cell which has not been so modified (or population of such cells). Preferably the cell is a Treg or the population of cells is a population of Tregs.

Techniques for measuring the levels of specific mRNA and protein are well known in the art. mRNA levels in a population of cells, such as Tregs, may be measured by techniques such as the Affymetrix ebioscience prime flow RNA assay, Northern blotting, serial analysis of gene expression (SAGE) or quantitative polymerase chain reaction (qPCR). Protein levels in a population of cells may be measured by techniques such as flow cytometry, high-performance liquid chromatography (HPLC), liquid chromatography-mass spectrometry (LC/MS), Western blotting or enzyme-linked immunosorbent assay (ELISA).

A “FOXP3 polypeptide” is a polypeptide having FOXP3 activity i.e., a polypeptide able to bind FOXP3 target DNA and function as a transcription factor regulating development and function of Tregs. Particularly, a FOXP3 polypeptide may have the same or similar activity to wildtype FOXP3 (SEQ ID NO. 129), e.g., may have at least 40, 50, 60, 70, 80, 90, 95, 100, 110, 120, 130, 140 or 150% of the activity of the wildtype FOXP3 polypeptide. Thus, a FOXP3 polypeptide encoded by the nucleotide sequence in the nucleic acid or vector described herein may have increased or decreased activity compared to wildtype FOXP3. Techniques for measuring transcription factor activity are well known in the art. For example, transcription factor DNA-binding activity may be measured by ChIP. The transcription regulatory activity of a transcription factor may be measured by quantifying the level of expression of genes which it regulates. Gene expression may be quantified by measuring the levels of mRNA and/or protein produced from the gene using techniques such as Northern blotting, SAGE, qPCR, HPLC, LC/MS, Western blotting or ELISA. Genes regulated by FOXP3 include cytokines such as IL-2, IL-4 and IFN-γ (Siegler et al. Annu. Rev. Immunol. 2006, 24: 209-26, incorporated herein by reference). As discussed in detail below, FOXP3 or a FOXP3 polypeptide includes functional fragments, variants, and isoforms thereof, e.g., of SEQ ID NO. 32.

A “functional fragment of FOXP3” may refer to a portion or region of a FOXP3 polypeptide or a polynucleotide (i.e., nucleotide sequence) encoding a FOXP3 polypeptide that has the same or similar activity to the full-length FOXP3 polypeptide or polynucleotide. The functional fragment may have at least 40%, at least 50%, at least 60%, at least 70%, at least 80%, at least 90%, at least 95%, or 100% of the activity of the full-length FOXP3 polypeptide or polynucleotide. A person skilled in the art would be able to generate functional fragments based on the known structural and functional features of FOXP3. These are described, for instance, in Song, X., et al., 2012. Cell reports, 1(6), pp. 665-675; Lopes, J. E., et al., 2006. The Journal of Immunology, 177(5), pp. 3133-3142; and Lozano, T., et al, 2013. Frontiers in oncology, 3, p. 294. Further, a N and C terminally truncated FOXP3 fragment is described within WO2019/241549 (incorporated herein by reference), for example, having the sequence SEQ ID NO. 37 as discussed below.

A “FOXP3 variant” may include an amino acid sequence or a nucleotide sequence which may be at least 50%, at least 55%, at least 65%, at least 70%, at least 75%, at least 80%, at least 85% or at least 90% identical, preferably at least 95% or at least 97% or at least 99% identical to a FOXP3 polypeptide or a polynucleotide encoding a FOXP3 polypeptide, e.g., to SEQ ID NO. 129. FOXP3 variants may have the same or similar activity to a wildtype FOXP3 polypeptide or polynucleotide, e.g., may have at least 40, 50, 60, 70, 80, 90, 95, 100, 110, 120, 130, 140 or 150% of the activity of a wildtype FOXP3 polypeptide or polynucleotide. A person skilled in the art would be able to generate FOXP3 variants based on the known structural and functional features of FOXP3 and/or using conservative substitutions. FOXP3 variants may have similar or the same turnover time (or degradation rate) within a Treg cell as compared to wildtype FOXP3, e.g., at least 40, 50, 60, 70, 80, 90, 95, 99 or 100% of the turnover time (or degradation rate) of wildtype FOXP3 in a Treg. Some FOXP3 variants may have a reduced turnover time (or degradation rate) as compared to wildtype FOXP3, for example, FOXP3 variants having amino acid substitutions at amino acid 418 and/or 422 of SEQ ID NO. 32, for example S418E and/or S422A, as described in WO2019/241549 (incorporated herein by reference).

Suitably, the FOXP3 polypeptide encoded by a nucleic acid molecule or vector as described herein may comprise or consist of the polypeptide sequence of a human FOXP3, such as UniProtKB accession Q9BZS1 (SEQ ID NO: 32), or a functional fragment or variant thereof.

In some embodiments of the invention, the FOXP3 polypeptide comprises or consists of an amino acid sequence which is at least 70% identical to SEQ ID NO: 32 or a functional fragment thereof. Suitably, the FOXP3 polypeptide comprises or consists of an amino acid sequence which is at least 80%, at least 85%, at least 90%, at least 95%, at least 98% or at least 99% identical to SEQ ID NO: 32 or a functional fragment thereof. In some embodiments, the FOXP3 polypeptide comprises or consists of SEQ ID NO: 32 or a functional fragment thereof.

In some embodiments, as discussed above, the FOXP3 polypeptide may comprise mutations at residues 418 and/or 422 of SEQ ID NO. 32.

In some embodiments of the invention, the FOXP3 polypeptide may be truncated at the N and/or C terminal ends, resulting in the production of a functional fragment. Particularly, an N and C terminally truncated functional fragment of FOXP3 may comprise or consist of an amino acid sequence of SEQ ID NO. 37 or a functional variant thereof having at least 80, 85, 90, 95 or 99% identity thereto.

Suitably, the FOXP3 polypeptide may be a variant of SEQ ID NO: 32, for example a natural variant. Suitably, the FOXP3 polypeptide is an isoform of SEQ ID NO: 32. For example, the FOXP3 polypeptide may comprise a deletion of amino acid positions 72-106 relative to SEQ ID NO: 32. Alternatively, the FOXP3 polypeptide may comprise a deletion of amino acid positions 246-272 relative to SEQ ID NO: 32.

The present nucleic acid molecule or construct may further comprise a nucleic acid sequence encoding a selectable marker. Suitably selectable markers are well known in the art and include, but are not limited to, fluorescent proteins—such as GFP. Suitably, the selectable marker may be a fluorescent protein, for example GFP, YFP, RFP, tdTomato, dsRed, or variants thereof. In some embodiments the fluorescent protein is GFP or a GFP variant.

Suitably, the selectable marker/reporter domain may be a luciferase-based reporter, a PET reporter (e.g. Sodium Iodide Symporter (NIS)), or a membrane protein (e.g. CD34, low-affinity nerve growth factor receptor (LNGFR)).

The use of a selectable marker is advantageous as it allows cells (e.g. Tregs) in which a nucleic acid molecule, construct or vector has been successfully introduced (such that the encoded chimeric protein and any other encoded proteins or polypeptides are expressed) to be selected and isolated from a starting cell population using common methods, e.g. flow cytometry.

In a still further embodiment, the recombinant protein may be co-expressed with a mutant calcineurin protein which is resistant to at least one calcineurin inhibitor, and in particular a mutant calcineurin protein which is resistant to at least one calcineurin inhibitor and sensitive to at least one calcineurin inhibitor. Such calcineurin mutants are discussed further below. In such an embodiment the nucleic acid molecule or construct may further comprise a nucleotide sequence encoding such a mutant calcineurin.

Where two or more coding sequences are expressed from a single nucleic acid molecule or construct, they may be linked by a sequence allowing co-expression of the two or more coding sequences. In particular, the co-expression sequence, or alternatively termed, the co-expression site, may enable expression of an encoded protein or polypeptide as a discrete entity. For example, the construct may comprise an internal promoter, an internal ribosome entry sequence (IRES) sequence or a sequence encoding a cleavage site.

In particular the co-expression sequence may encode a self-cleavage sequence in between encoded polypeptides. Particularly, the self-cleaving sequence may be a self-cleaving peptide. Such sequences auto-cleave during protein production. Self-cleaving peptides which may be used are 2A peptides or 2A-like peptides which are known and described in the art, for example in Donnelly et al., Journal of General Virology, 2001, 82, 1027-1041, herein incorporated by reference. 2A and 2A-like peptides are believed to cause ribosome skipping, and result in a form of cleavage in which a ribosome skips the formation of peptide bond between the end of a 2A peptide and the downstream amino acid sequence. The “cleavage” occurs between the Glycine and Proline residues at the C-terminus of the 2A peptide meaning the upstream cistron will have a few additional residues added to the end, while the downstream cistron will start with the Proline.

Suitable self-cleaving domains include P2A, T2A, E2A, and F2A sequences as shown in SEQ ID NO: 100-103 respectively. The sequences may be modified to include the amino acids GSG at the N-terminus of the 2A peptides. Thus, also included as possible options are sequences corresponding to SEQ ID NOs. 100-103, but with GSG at the N termini thereof. Such modified alternative 2A sequences are known and reported in the art. Alternative 2A-like sequences which may be used are shown in Donnelly et al (supra), for example a TaV sequence.

The self-cleaving sequences included in the nucleic acid molecule may be the same or different.

The self-cleaving sequence may include an additional cleavage site, which may be cleaved by common enzymes present in the cell. This may assist in achieving complete removal of the 2A sequences after translation. Such an additional cleavage site may for example comprise a Furin cleavage site. Such cleavage sites are known in the art, and may include for example RXXR (SEQ ID NO: 104), for example RRKR (SEQ ID NO: 105).

The nucleic acid molecule/polynucleotides used herein may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

The nucleotide sequence encoding the recombinant protein, and any other coding nucleotide sequences may be provided in a construct in which they are operably linked to a promoter. In some cases, different nucleotide sequences may be operably linked to the same promoter. A “promoter” is a region of DNA that leads to initiation of transcription of a gene. Promoters are located near the transcription start sites of genes, upstream on the DNA (towards the 5′ region of the sense strand). Any suitable promoter may be used, the selection of which may be readily made by the skilled person. The promoter may be from any source, and may be a viral promoter, or a eukaryotic promoter, including mammalian or human promoters (i.e. a physiological promoter). In an embodiment the promoter is a viral promoter. Particular promoters include LTR promoters, EFS (or functional truncations thereof), SFFV, PGK, and CMV. In an embodiment the promoter is SFFV or a viral LTR promoter. “Operably linked to the same promoter” means that transcription of the polynucleotide sequences may be initiated from the same promoter and that the nucleotide sequences are positioned and oriented for transcription to be initiated from the promoter. Polynucleotides operably linked to a promoter are under transcriptional regulation of the promoter.

A vector is a tool that allows or facilitates the transfer of an entity from one environment to another. As used herein, and by way of example, some vectors used in recombinant nucleic acid techniques allow entities, such as a segment of nucleic acid (e.g. a heterologous DNA segment, such as a heterologous cDNA segment), to be transferred into a target cell. Vectors may be non-viral or viral. Examples of vectors used in recombinant nucleic acid techniques include, but are not limited to, plasmids, mRNA molecules (e.g. in vitro transcribed mRNAs), chromosomes, artificial chromosomes and viruses. The vector may also be, for example, a naked nucleic acid (e.g. DNA). In its simplest form, the vector may itself be a nucleotide sequence of interest.

The vectors used herein may be, for example, plasmid, mRNA or virus vectors and may include a promoter (as described above) for the expression of a nucleic acid molecule/polynucleotide and optionally a regulator of the promoter.

In an embodiment the vector is a viral vector, for example a retroviral, e.g. a lentiviral vector or a gamma retroviral vector.

The vectors may further comprise additional promoters, for example, in one embodiment, the promoter may be a LTR, for example, a retroviral LTR or a lentiviral LTR. Long terminal repeats (LTRs) are identical sequences of DNA that repeat hundreds or thousands of times found at either end of retrotransposons or proviral DNA formed by reverse transcription of retroviral RNA. They are used by viruses to insert their genetic material into the host genomes. Signals of gene expression are found in LTRs: enhancer, promoter (can have both transcriptional enhancers or regulatory elements), transcription initiation (such as capping), transcription terminator and polyadenylation signal.

Suitably, the vector may include a 5′LTR and a 3′LTR.

The vector may comprise one or more additional regulatory sequences which may act pre- or post-transcriptionally. “Regulatory sequences” are any sequences which facilitate expression of the polypeptides, e.g. act to increase expression of a transcript or to enhance mRNA stability. Suitable regulatory sequences include for example enhancer elements, post-transcriptional regulatory elements and polyadenylation sites. Suitably, the additional regulatory sequences may be present in the LTR(s).

Suitably, the vector may comprise a Woodchuck Hepatitis Virus Posttranscriptional Regulatory Element (WPRE), e.g. operably linked to the promoter.

Vectors comprising the present nucleic acid molecules/polynucleotides may be introduced into cells using a variety of techniques known in the art, such as transformation and transduction. Several techniques are known in the art, for example infection with recombinant viral vectors, such as retroviral, lentiviral, adenoviral, adeno-associated viral, baculoviral and herpes simplex viral vectors; direct injection of nucleic acids and biolistic transformation.

Non-viral delivery systems include but are not limited to DNA transfection methods. Here, transfection includes a process using a non-viral vector to deliver a gene to a target cell. Non-viral delivery systems can include liposomal or amphipathic cell penetrating peptides, preferably complexed with a nucleic acid molecule or construct.

Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14: 556) and combinations thereof.

In some cases, the present nucleic acid molecules may be designed to be used as single constructs which encode the recombinant protein and any other polypeptide (e.g. receptor or marker or other functional polypeptide or protein of interest) and this would be contained in a single vector, it is not precluded that they are introduced into a cell in conjunction with other vectors, for example encoding other polypeptides it may be desired also to introduce into the cell.

As noted above, the recombinant protein may be co-expressed in or on a cell in conjunction with a CAR. The term “chimeric antigen receptor” or “CAR” as used herein refers to engineered receptors which can confer an antigen specificity onto cells (for example Tregs). CARs are also known as artificial T-cell receptors, chimeric T-cell receptors or chimeric immunoreceptors. A CAR typically comprises an extracellular domain comprising an antigen-specific targeting region, termed herein an antigen-binding domain, a transmembrane domain, and an intracellular domain comprising optionally one or more costimulatory domains, and an intracellular signaling domain. The antigen-binding domain is typically joined to the transmembrane domain by a hinge domain. The design of CARs, and the various domains that they may contain, is well known in the art.

When the CAR binds its target antigen, this results in the transmission of an activating signal to the cell in which it is expressed. Thus, the CAR directs the specificity of the engineered cells towards the target antigen, particularly towards cells expressing the targeted antigen.

The antigen-binding domain of a CAR may be derived or obtained from any protein or polypeptide which binds (i.e. has affinity for) a desired target antigen, or more generally a desired target molecule. This may be for example, a ligand or receptor, or a physiological binding protein for the target molecule, or a part thereof, or a synthetic or derivative protein. The target molecule may commonly be expressed on the surface of a cell, for example a target cell, or a cell in the vicinity of a target cell (for a bystander effect), but need not be. Depending on the nature and specificity of the antigen binding domain, the CAR may recognise a soluble molecule, for example where the antigen-binding domain is based on, or derived from, a cellular receptor.

The antigen-binding domain is most commonly derived from antibody variable chains (for example it commonly takes the form of a scFv), but may also be generated from T-cell receptor variable domains or, as mentioned above, other molecules, such as receptors for ligands or other binding molecules.

The CAR is typically expressed as a polypeptide also comprising a signal sequence (also known as a leader sequence)), and in particular a signal sequence which targets the CAR to the plasma membrane of the cell. This will generally be positioned next to or close to the antigen-binding domain, generally upstream of the antigen-binding domain. The extracellular domain, or ectodomain, of the CAR may thus comprise a signal sequence and an antigen-binding domain.

The antigen-binding domain provides the CAR with the ability to bind a predetermined antigen of interest. The antigen-binding domain preferably targets an antigen of clinical interest or an antigen at a site of disease.

As noted above, the antigen-binding domain may be any protein or peptide that possesses the ability to specifically recognize and bind to a biological molecule (e.g., a cell surface receptor or a component thereof). The antigen-binding domain includes any naturally occurring, synthetic, semi-synthetic, or recombinantly produced binding partner for a biological molecule of interest. Illustrative antigen-specific targeting domains include antibodies or antibody fragments or derivatives, extracellular domains of receptors, ligands for cell surface molecules/receptors, or receptor binding domains thereof, and tumor binding proteins. Although as discussed below, the antigen-specific targeting domain may preferably be an antibody or derived from an antibody, other antigen-specific targeting domains are encompassed, e.g. antigen-specific targeting domains formed from an antigenic peptide/MHC or HLA combination which is capable of binding to the TCRs of Tcon cells active at a site of transplantation, inflammation or disease.

The CAR may be directed towards any desired target antigen or molecule. This may be selected according to the intended therapy, and the condition it is desired to treat. It may for example be an antigen or molecule associated with a particular condition, or an antigen or molecule associated with a cell it is desired to target to treat the condition. Typically, the antigen or molecule is a cell-surface antigen or molecule.

The term “directed against” is synonymous with “specific for” or “anti”. Put another way, the CAR recognises a target molecule. Accordingly, it is meant that the CAR is capable of binding specifically to a specified or given antigen, or target. In particular, the antigen-binding domain of the CAR is capable of binding specifically to the target molecule or antigen (more particularly when the CAR is expressed on the surface of a cell, notably an immune effector cell). Specific binding may be distinguished from non-specific binding to a non-target molecule or antigen. Thus, a cell expressing the CAR is directed, or re-directed, to bind specifically to a target cell, expressing the target molecule or antigen, particularly a target cell expressing the target antigen or molecule on its cell surface.

Antigens which may be targeted by the present CAR include, but are not limited to, antigens expressed on cells associated with transplanted organs, autoimmune diseases, allergic diseases and inflammatory diseases (e.g. neurodegenerative disease). It will be understood by a skilled person that where the cell engineered to express the CAR is a Treg cell, or a precursor therefor, due to the bystander effect of Treg cells, the antigen may be simply present and/or expressed at the site of transplantation, inflammation or disease.

Antigens expressed on cells associated with neurodegenerative disease include those presented on glial cells, e.g. MOG.

Antigens associated with organ transplants and/or cells associated with transplanted organs include, but are not limited to, a HLA antigen present in the transplanted organ but not in the patient, or an antigen whose expression is up-regulated during transplant rejection such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11.

In an embodiment the CAR is directed against an HLA antigen, and in particular an HLA-A2 antigen.

Antibodies against such antigens and are known in the art, and conveniently a scFv may be obtained or generated bases on a known or available antibody. In this regard VH and VL, and CDR sequences are publically available to aid the preparation of such an antibody-binding domain, for example in WO 2020/044055, the disclosure of which is herein incorporated by reference. Any of the antigen binding domains, or CDR, VH, and/or VL sequences disclosed in WO 2020/044055 may be used.

By way of example, the CAR may comprise an antigen binding domain which is capable of binding HLA-A2 (HLA-A2 may also be referred to herein as HLA-A*02, HLA-A02, and HLA-A*2). HLA-A*02 is one particular class I major histocompatibility complex (MHC) allele group at the HLA-A locus.

The antigen recognition domain may bind, suitably specifically bind, one or more regions or epitopes within HLA-A2. An epitope, also known as antigenic determinant, is the part of an antigen that is recognised by an antigen recognition domain (e.g. an antibody). In other words, the epitope is the specific piece of the antigen to which an antibody binds. Suitably, the antigen recognition domain binds, suitably specifically binds, to one region or epitope within HLA-A2.

Engineered cells, particularly T cells, may be generated by introducing a nucleic acid molecule, construct, or vector as defined herein, by one of many means including transduction with a viral vector, and transfection with DNA or RNA.

The present cell may be made by: introducing to a cell (e.g. by transduction or transfection) the nucleic acid molecule, construct or vector as defined herein.

Suitable cells are discussed further below, but the cell may be from a sample isolated from a subject. The subject may be a donor subject, or a subject for therapy (i.e. the cell may be an autologous cell, or a donor cell, for introduction to another recipient, e.g. an allogeneic cell).

The cell may be generated by a method comprising the following steps:
(i) isolation of a cell-containing sample from a subject or provision of a cell-containing sample; and
(ii) introduction into (e.g. by transduction or transfection) the cell-containing sample of a nucleic acid molecule, construct, or vector as defined herein, to provide a population of engineered cells.

A cell into which a nucleic acid molecule, construct or vector is to be introduced may be referred to as a target cell. A target cell-enriched sample may be isolated from, enriched, and/or generated from the cell-containing sample prior to and/or after step (ii) of the method. For example, isolation, enrichment and/or generation of Tregs (or other target cells) may be performed prior to and/or after step (ii) to isolate, enrich or generate a Treg-enriched sample. Isolation and/or enrichment from a cell-containing sample may be performed after step (ii) to enrich for cells and/or Tregs (or other target cells) comprising the CAR, the nucleic acid molecule/polynucleotide, the construct and/or the vector as described herein.

A Treg-enriched sample may be isolated or enriched by any method known to those of skill in the art, for example by FACS and/or magnetic bead sorting. A Treg-enriched sample may be generated from the cell-containing sample by any method known to those of skill in the art, for example, from Tcon cells by introducing DNA or RNA coding for FOXP3 and/or from ex-vivo differentiation of inducible progenitor cells or embryonic progenitor cells. Methods for isolating and/or enriching other target cells are known in the art.

The target cell may be a Treg cell, or precursor or a progenitor therefor.

An “engineered cell” means a cell which has been modified to comprise or express a polynucleotide which is not naturally encoded by the cell. Methods for engineering cells are known in the art and include, but are not limited to, genetic modification of cells e.g. by transduction such as retroviral or lentiviral transduction, transfection (such as transient transfection—DNA or RNA based) including lipofection, polyethylene glycol, calcium phosphate and electroporation, as discussed above. Any suitable method may be used to introduce a nucleic acid sequence into a cell. Non-viral technologies such as amphipathic cell penetrating peptides may be used to introduce nucleic acid. A cell may also be genetically modified e.g. using any known gene editing technique to insert a nucleotide, polynucleotide or nucleic acid sequence as described herein into the genome, e.g. using CRISPR, Talens or Zn fingers.

Accordingly, the nucleic acid molecule as described herein is not naturally expressed by a corresponding, unmodified cell. Indeed, the nucleic acid molecule encoding the recombinant protein is an artificial construct, and could not occur or be expressed naturally. Suitably, an engineered cell is a cell which has been modified e.g. by transduction or by transfection. Suitably, an engineered cell is a cell which has been modified or whose genome has been modified e.g. by transduction or by transfection. Suitably, an engineered cell is a cell which has been modified or whose genome has been modified by retroviral transduction. Suitably, an engineered cell is a cell which has been modified or whose genome has been modified by lentiviral transduction.

As used herein, the term “introduced” refers to methods for inserting foreign nucleic acid, e.g. DNA or RNA, into a cell. As used herein the term introduced includes both transduction and transfection methods. Transfection is the process of introducing nucleic acids into a cell by non-viral methods. Transduction is the process of introducing foreign DNA or RNA into a cell via a viral vector. Engineered cells may be generated by introducing a nucleic acid as described herein by one of many means including transduction with a viral vector, transfection with DNA or RNA.

Cells may be activated and/or expanded prior to, or after, the introduction of a nucleic acid as described herein, for example by treatment with an anti-CD3 monoclonal antibody or both anti-CD3 and anti-CD28 monoclonal antibodies. The cells may also be expanded in the presence of anti-CD3 and anti-CD28 monoclonal antibodies in combination with IL-2. Suitably, IL-2 may be substituted with IL-15. Other components which may be used in a cell (e.g. Treg) expansion protocol include, but are not limited to rapamycin, all-trans retinoic acid (ATRA) and TGFβ. As used herein “activated” means that a cell has been stimulated, causing the cell to proliferate. As used herein “expanded” means that a cell or population of cells has been induced to proliferate. The expansion of a population of cells may be measured for example by counting the number of cells present in a population. The phenotype of the cells may be determined by methods known in the art such as flow cytometry. In one embodiment, cells may be cultured and/or activated in the presence of EPO.

The cell may be an immune cell, or a precursor therefor. A precursor cell may be a progenitor cell. Representative immune cells thus include T-cells, in particular, cytotoxic T-cells (CTLs; CD8+ T-cells), helper T-cells (HTLs; CD4+ T-cells) and regulatory T cells (Tregs). Other populations of T-cells are also useful herein, for example naive T-cells and memory T-cells. Other immune cells include NK cells, NKT cells, dendritic cells, MDSC, neutrophils, and macrophages. Precursors of immune cells include pluripotent stem cells, e.g. induced PSC (iPSC), or more committed progenitors including multipotent stem cells, or cells which are committed to a lineage. Precursor cells can be induced to differentiate into immune cells in vivo or in vitro. In one aspect, a precursor cell may be a somatic cell which is capable of being transdifferentiated to an immune cell of interest.

Most notably, the immune cell may be an NK cell, a dendritic cell, a MDSC, or a T cell, such as a cytotoxic T lymphocyte (CTL) or a Treg cell.

In particular, the immune cell may be a Treg cell. “Regulatory T cells (Treg) or T regulatory cells” are immune cells with immunosuppressive function that control cytopathic immune responses and are essential for the maintenance of immunological tolerance. As used herein, the term Treg refers to a T cell with immunosuppressive function.

A T cell as used herein is a lymphocyte including any type of T cell, such as an alpha beta T cell (e.g. CD8 or CD4+), a gamma delta T cell, a memory T cell, a Treg cell.

Suitably, immunosuppressive function may refer to the ability of the Treg to reduce or inhibit one or more of a number of physiological and cellular effects facilitated by the immune system in response to a stimulus such as a pathogen, an alloantigen, or an autoantigen. Examples of such effects include increased proliferation of conventional T cell (Tcon) and secretion of proinflammatory cytokines. Any such effects may be used as indicators of the strength of an immune response. A relatively weaker immune response by Tconv in the presence of Tregs would indicate an ability of the Treg to suppress immune responses. For example, a relative decrease in cytokine secretion would be indicative of a weaker immune response, and thus indicative of the ability of Tregs to suppress immune responses. Tregs can also suppress immune responses by modulating the expression of co-stimulatory molecules on antigen presenting cells (APCs), such as B cells, dendritic cells and macrophages. Expression levels of CD80 and CD86 can be used to assess suppression potency of activated Tregs in vitro after co-culture.

Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured Tregs.

Antigen-specific Tconv cells co-cultured with Tregs as referred to herein may proliferate 5%, 10%, 20%, 30%, 40%, 50%, 60%, 70%, 90%, 95% or 99% less than the same Tconv cells cultured in the absence of the Tregs. For example, antigen-specific Tconv cells co-cultured with the present Tregs may proliferate 5%, 10%, 20%, 30%, 40%, 50%, 60%, 70%, 90%, 95% or 99% less than the same Tconv cells cultured in the presence of non-engineered Tregs. The cells comprising the nucleic acid, expression construct or vector as defined herein, e.g. Tregs may have an increased suppressive activity as compared to non-engineered Tregs (e.g. an increased suppressive activity of at least 5, 10, 20, 30, 40, 50, 60, 70, 80 or 90%).

Antigen-specific Tconv cells co-cultured with the Tregs herein may express at least 10%, at least 20%, at least 30%, at least 40%, at least 50%, or at least 60% less effector cytokine than corresponding Tconv cells cultured in the absence of the Tregs (e.g. in the presence of non-engineered Tregs). The effector cytokine may be selected from IL-2, IL-17, TNFα, GM-CSF, IFN-γ, IL-4, IL-5, IL-9, IL-10 and IL-13. Suitably the effector cytokine may be selected from IL-2, IL-17, TNFα, GM-CSF and IFN-γ.

Several different subpopulations of Tregs have been identified which may express different or different levels of particular markers. Tregs generally are T cells which express the markers CD4, CD25 and FOXP3 (CD4⁺CD25⁺FOXP3⁺).

Tregs may also express CTLA-4 (cytotoxic T-lymphocyte associated molecule-4) or GITR (glucocorticoid-induced TNF receptor).

Treg cells are present in the peripheral blood, lymph nodes, and tissues and Tregs for use herein include thymus-derived, natural Treg (nTreg) cells, peripherally generated Tregs, and induced Treg (iTreg) cells.

A Treg may be identified using the cell surface markers CD4 and CD25 in the absence of or in combination with low-level expression of the surface protein CD127 (CD4⁺CD25⁺CD127⁻ or CD4⁺CD25⁺CD127^{low}). The use of such markers to identify Tregs is known in the art and described in Liu et al. (JEM; 2006; 203; 7(10); 1701-1711), for example.

A Treg may be a CD4⁺CD25⁺FOXP3⁺ T cell, a CD4⁺CD25⁺CD127⁻ T cell, or a CD4⁺CD25⁺FOXP3⁺CD127^{-/low} T cell.

Suitably, the Treg may be a natural Treg (nTreg). As used herein, the term “natural T reg” means a thymus-derived Treg. Natural T regs are CD4⁺CD25⁺FOXP3⁺ Helios⁺ Neuropilin 1⁺. Compared with iTregs, nTregs have higher expression of PD-1 (programmed cell death-1, pdcd1), neuropilin 1 (Nrp1), Helios (Ikzf2), and CD73. nTregs may be distinguished from iTregs on the basis of the expression of Helios protein or Neuropilin 1 (Nrp1) individually.

The Treg may have a demethylated Treg-specific demethylated region (TSDR). The TSDR is an important methylation-sensitive element regulating Foxp3 expression (Polansky, J. K., et al., 2008. European journal of immunology, 38(6), pp. 1654-1663).

Further suitable Tregs include, but are not limited to, Tr1 cells (which do not express Foxp3, and have high IL-10 production); CD8+FOXP3⁺ T cells; and γδ FOXP3′ T cells.

Different subpopulations of Tregs are known to exist, including naïve Tregs (CD45RA⁺FoxP3^{low}), effector/memory Tregs (CD45RA-FoxP3^{high}) and cytokine-producing Tregs (CD45RA-FoxP3^{low}). “Memory Tregs” are Tregs which express CD45RO and which are considered to be CD45RO⁺. These cells have increased levels of CD45RO as compared to naïve Tregs (e.g. at least 10, 20, 30, 40, 50, 60, 70, 80 or 90% more CD45RO) and which preferably do not express or have low levels of CD45RA (mRNA and/or protein) as compared to naïve Tregs (e.g. at least 80, 90 or 95% less CD45RA as compared to naïve Tregs). “Cytokine-producing Tregs” are Tregs which do not express or have very low levels of CD45RA (mRNA and/or protein) as compared to naïve Tregs (e.g. at least 80, 90 or 95% less CD45RA as compared to naïve Tregs), and which have low levels of FOXP3 as compared to Memory Tregs, e.g. less than 50, 60, 70, 80 or 90% of the FOXP3 as compared to Memory Tregs. Cytokine-producing Tregs may produce interferon gamma and may be less suppressive in vitro as compared to naïve Tregs (e.g. less than 50, 60, 70, 80 or 90% suppressive than naïve Tregs. Reference to expression levels herein may refer to mRNA or protein expression. Particularly, for cell surface markers such as CD45RA, CD25, CD4, CD45RO etc., expression may refer to cell surface expression, i.e. the amount or relative amount of a marker protein that is expressed on the cell surface. Expression levels may be determined by any known method of the art. For example, mRNA expression levels may be determined by Northern blotting/array analysis, and protein expression may be determined by Western blotting, or preferably by FACS using antibody staining for cell surface expression.

Particularly, the Treg may be a naïve Treg. “A naïve regulatory T cell, a naïve T regulatory cell, or a naïve Treg” as used interchangeably herein refers to a Treg cell which expresses CD45RA (particularly which expresses CD45RA on the cell surface). Naïve Tregs are thus described as CD45RA⁺. Naïve Tregs generally represent Tregs which have not been activated through their endogenous TCRs by peptide/MHC, whereas effector/memory Tregs relate to Tregs which have been activated by stimulation through their endogenous TCRs. Typically, a naïve Treg may express at least 10, 20, 30, 40, 50, 60, 70, 80 or 90% more CD45RA than a Treg cell which is not naïve (e.g. a memory Treg cell). Alternatively viewed, a naïve Treg cell may express at least 2, 3, 4, 5, 10, 50 or 100-fold the amount of CD45RA as compared to a non-naïve Treg cell (e.g. a memory Treg cell). The level of expression of CD45RA can be readily determined by methods of the art, e.g. by flow cytometry using commercially available antibodies. Typically, non-naïve Treg cells do not express CD45RA or low levels of CD45RA.

Particularly, naïve Tregs may not express CD45RO, and may be considered to be CD45RO⁻. Thus, naïve Tregs may express at least 10, 20, 30, 40, 50, 60, 70, 80 or 90% less CD45RO as compared to a memory Treg, or alternatively viewed at least 2, 3, 4, 5, 10, 50 or 100 fold less CD45RO than a memory Treg cell.

Although naïve Tregs express CD25 as discussed above, CD25 expression levels may be lower than expression levels in memory Tregs, depending on the origin of the naïve Tregs. For example, for naïve Tregs isolated from peripheral blood, expression levels of CD25 may be at least 10, 20, 30, 40, 50, 60, 70, 80 or 90% lower than memory Tregs. Such naïve Tregs may be considered to express intermediate to low levels of CD25. However, a skilled person will appreciate that naïve Tregs isolated from cord blood may not show this difference.

Typically, a naïve Treg as defined herein may be CD4⁺, CD25⁺, FOXP3⁺, CD127^{low}, CD45RA⁺.

Low expression of CD127 as used herein refers to a lower level of expression of CD127 as compared to a CD4⁺ non-regulatory or Tcon cell from the same subject or donor. Particularly, naïve Tregs may express less than 90, 80, 70, 60, 50, 40, 30, 20 or 10% CD127 as compared to a CD4⁺ non-regulatory or Tcon cell from the same subject or donor. Levels of CD127 can be assessed by methods standard in the art, including by flow cytometry of cells stained with an anti-CD127 antibody.

Typically, naïve Tregs do not express, or express low levels of CCR4, HLA-DR, CXCR3 and/or CCR6. Particularly, naïve Tregs may express lower levels of CCR4, HLA-DR, CXCR3 and CCR6 than memory Tregs, e.g. at least 10, 20, 30, 40, 50, 60, 70, 80 or 90% lower level of expression.

Naïve Tregs may further express additional markers, including CCR7⁺ and CD31⁺

Isolated naïve Tregs may be identified by methods known in the art, including by determining the presence or absence of a panel of any one or more of the markers discussed above, on the cell surface of the isolated cells. For example, CD45RA, CD4, CD25 and CD127 low can be used to determine whether a cell is a naïve Treg. Methods of determining whether isolated cells are naïve Tregs or have a desired phenotype can be carried out as discussed below in relation to additional steps which may be carried out, and methods for determining the presence and/or levels of expression of cell markers are well-known in the art and include, for example, flow cytometry, using commercially available antibodies.

Suitably, the cell, such as a Treg, is isolated from peripheral blood mononuclear cells (PBMCs) obtained from a subject. Suitably the subject from whom the PBMCs are obtained is a mammal, preferably a human. Suitably the cell is matched (e.g. HLA matched) or is autologous to the subject to whom the engineered cell is to be administered. Suitably, the subject to be treated is a mammal, particularly a human. The cell may be generated ex vivo either from a patient's own peripheral blood (1st party), or in the setting of a haematopoietic stem cell transplant from donor peripheral blood (2nd party), or peripheral blood from an unconnected donor (3rd party). Suitably the cell is autologous to the subject to whom the engineered cell is to be administered.

Suitably, the Treg is part of a population of cells. Suitably, the population of Tregs comprises at least 70% Tregs, such as at least 75, 85, 90, 95, 97, 98 or 99% Tregs. Such a population may be referred to as an “enriched Treg population”.

In some aspects, the Treg may be derived from ex-vivo differentiation of inducible progenitor cells (e.g. iPSCs) or embryonic progenitor cells to the Treg. A nucleic acid molecule, construct or vector as described herein may be introduced into the inducible progenitor cells or embryonic progenitor cells prior to, or after, differentiation to a Treg. Suitable methods for differentiation are known in the art and include that disclosed in Haque et al, J Vis Exp., 2016, 117, 54720 (incorporated herein by reference). In another aspect, where the recombinant protein is a EPOR with a modification providing for dimerisation and signalling in the absence of a signal inducer molecule, the endogenous EPOR nucleic acid may be modified by gene editing techniques (e.g. CRISPR) to provide a cell of the invention.

As used herein, the term “conventional T cell” or Tcon or Tconv (used interchangeably herein) means a T lymphocyte cell which expresses an as T cell receptor (TCR) as well as a co-receptor which may be cluster of differentiation 4 (CD4) or cluster of differentiation 8 (CD8) and which does not have an immunosuppressive function. Conventional T cells are present in the peripheral blood, lymph nodes, and tissues. Suitably, the engineered Treg may generated from a Tcon by introducing the nucleic acid which includes a sequence coding for FOXP3 Alternatively, the engineered Treg may be generated from a Tcon by in vitro culture of CD4+ CD25−FOXP3− cells in the presence of IL-2 and TGF-β.

When the recombinant protein is expressed, a Treg herein may have increased persistence as compared to a Treg cell without the recombinant protein. “Persistence” as used herein defines the length of time that Tregs can survive in a particular environment, e.g. in vivo (e.g. in a human patient or animal model). A Treg as disclosed herein may have at least 10, 20, 30, 40, 50, 60, 70, 80 or 90% increased persistence as compared to a Treg which does not express the recombinant protein herein. Persistence can be measured by for example, determining the amount or numbers of administered cells within a subject or patient over time, where cells expressing a recombinant protein of the invention are compared to equivalent cell types which do not express the recombinant protein, or compared to non-engineered cells. It is possible to track administered cells, for example, using a marker protein, e.g. CD34 for cells which also express a RQR8 safety switch.

In another embodiment the target cell into which the nucleic acid molecule, construct or vector is introduced is not a cell intended for therapy. In an embodiment the cell is a production host cell. The cell may be for production of the nucleic acid, e.g. cloning, or vector, or polypeptides.

Also provided herein is a cell population comprising a cell as defined or described herein. It will be appreciated that a cell population may comprise both the present cells comprising a nucleic acid molecule, construct or vector as defined herein, and cells which do not comprise the nucleic acid molecule, construct or vector, e.g. untransduced or untransfected cells. Although in a particular embodiment, all the cells in a population may comprise the nucleic acid, expression construct or vector, cell populations having at least 10, 20, 30, 40, 50, 60, 70, 80, 90, 95 or 99% of cells comprising a nucleic acid, expression construct or vector are provided.

There is also provided a pharmaceutical composition comprising a cell or cell population as defined or described herein, a vector as defined herein. The vector may be used for gene therapy. Thus, rather than administering a cell, a vector may be administered instead, to modify endogenous cells in the subject to express the introduced nucleic acid molecule. Vectors suitable for use in gene therapy are known in the art, and include viral vectors.

A pharmaceutical composition is a composition that comprises or consists of a therapeutically effective amount of a pharmaceutically active agent i.e. the cell (e.g. Treg), cell population or vector. It preferably includes a pharmaceutically acceptable carrier, diluent or excipient (including combinations thereof). Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s) or solubilising agent(s).

By “pharmaceutically acceptable” is included that the formulation is sterile and pyrogen free. The carrier, diluent, and/or excipient must be “acceptable” in the sense of being compatible with the cell or vector and not deleterious to the recipients thereof. Typically, the carriers, diluents, and excipients will be saline or infusion media which will be sterile and pyrogen free, however, other acceptable carriers, diluents, and excipients may be used.

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The cells, cell population or pharmaceutical compositions may be administered in a manner appropriate for treating and/or preventing the desired disease or condition. The quantity and frequency of administration will be determined by such factors as the condition of the subject, and the type and severity of the subject's disease or condition, although appropriate dosages may be determined by clinical trials. The pharmaceutical composition may be formulated accordingly.

The cell, cell population or pharmaceutical composition as described herein can be administered parenterally, for example, intravenously, or they may be administered by infusion techniques. The cell, cell population or pharmaceutical composition may be administered in the form of a sterile aqueous solution which may contain other substances, for example, enough salts or glucose to make the solution isotonic with blood. The aqueous solution may be suitably buffered (preferably to a pH of from 3 to 9). The pharmaceutical composition may be formulated accordingly. The preparation of suitable parenteral formulations under sterile conditions is readily accomplished by standard pharmaceutical techniques well-known to those skilled in the art.

The pharmaceutical compositions may comprise cells in infusion media, for example sterile isotonic solution. The pharmaceutical composition may be enclosed in ampoules, disposable syringes or multiple dose vials made of glass or plastic.

The cell, cell population or pharmaceutical composition may be administered in a single or in multiple doses. Particularly, the cell, cell population or pharmaceutical composition may be administered in a single, one off dose. The pharmaceutical composition may be formulated accordingly.

The pharmaceutical composition may further comprise one or more active agents. The pharmaceutical composition may further comprise one or more other therapeutic agents, such as lympho-depletive agents (e.g. thymoglobulin, campath-1H, anti-CD2 antibodies, anti-CD3 antibodies, anti-CD20 antibodies, cyclophosphamide, fludarabine), inhibitors of mTOR (e.g. sirolimus, everolimus), drugs inhibiting costimulatory pathways (e.g. anti-CD40/CD40L, CTAL41g), and/or drugs inhibiting specific cytokines (IL-6, IL-17, TNFalpha, IL18).

Depending upon the disease/condition and subject to be treated, as well as the route of administration, the cell, cell population or pharmaceutical composition may be administered at varying doses (e.g. measured in cells/kg or cells/subject). The physician in any event will determine the actual dosage which will be most suitable for any individual subject and it will vary with the age, weight and response of the particular subject. Typically, however, for the cells herein, doses of 5×10⁷ to 3×10⁹ cells, or 108 to 2×10⁹ cells per subject may be administered.

The cell may be appropriately modified for use in a pharmaceutical composition. For example, cells may be cryopreserved and thawed at an appropriate time, before being infused into a subject.

Further provided herein is the use of kits, or combination products, comprising the cell, cell population and/or pharmaceutical composition herein. Preferably said kits are for use in the methods and uses as described herein, e.g., the therapeutic methods as described herein. Preferably said kits comprise instructions for use of the kit components. Kits or compositions may further comprise the inducer, e.g. rapamycin or an analogue thereof.

The cells, cell populations, compositions and vectors herein may be for use therapy, that is in treating or preventing a disease or condition. As noted above, the cell in or on which the recombinant protein is expressed is typically a cell which is modified, or engineered to express a further molecule (e.g. a further protein), notably a receptor, e.g. a CAR or TCR, Accordingly, the therapy may be for the prevention or treatment of a disease or condition which may be treated by or with cell expressing the receptor, e.g. the CAR. The cells and compositions containing them are for adoptive cell therapy (ACT). Various conditions may be treated by administration of cells, including particularly Treg cells, expressing a CAR according to the present disclosure. As noted above, this may be conditions responsive to immunosuppression, and particularly the immunosuppressive effects of Tregs cells. The cells, cell populations, compositions and vectors described herein may thus be used for inducing, or achieving, immunosuppression in a subject. The Treg cells administered, or modified in vivo, may be targeted by expression of the receptor, e.g. CAR. Conditions suitable for such treatment include infectious, neurodegenerative or inflammatory disease, or more broadly a condition associated with any undesired or unwanted or deleterious immune response.

Conditions to be treated or prevented include inflammation, or alternatively put, a condition associated with or involving inflammation. Inflammation may be chronic or acute. Furthermore, the inflammation may be low-level or systemic inflammation. For example the inflammation may be inflammation which occurs in the context of a metabolic disorder, for example metabolic syndrome, or in the context of insulin resistance, or type II diabetes or obesity and such like.

In particular, the cells, cell populations, vectors and pharmaceutical compositions provide a means for inducing tolerance to a transplant; treating and/or preventing cellular and/or humoral transplant rejection; treating and/or preventing graft-versus-host disease (GvHD), an autoimmune or allergic disease; or to promote tissue repair and/or tissue regeneration; or to ameliorate inflammation. The cells, cell populations, vectors and pharmaceutical compositions may be used in a method which comprises the step of administering a cell, cell populations, vector or a pharmaceutical composition as described herein to a subject.

As used herein, “inducing tolerance to a transplant” refers to inducing tolerance to a transplanted organ in a recipient. In other words, inducing tolerance to a transplant means to reduce the level of a recipient's immune response to a donor transplant organ. Inducing tolerance to a transplanted organ may reduce the amount of immunosuppressive drugs that a transplant recipient requires, or may enable the discontinuation of immunosuppressive drugs.

For example, the engineered cells, e.g. Tregs, may be administered to a subject with a disease in order to lessen, reduce, or improve at least one symptom of disease such as jaundice, dark urine, itching, abdominal swelling or tenderness, fatigue, nausea or vomiting, and/or loss of appetite. The at least one symptom may be lessened, reduced, or improved by at least 10%, at least 20%, at least 30%, at least 40%, or at least 50%, or the at least one symptom may be completely alleviated.

The engineered cells, e.g. Tregs may be administered to a subject with a disease in order to slow down, reduce, or block the progression of the disease. The progression of the disease may be slowed down, reduced, or blocked by at least 10%, at least 20%, at least 30%, at least 40%, or at least 50% compared to a subject in which the engineered cells are not administered, or progression of the disease may be completely stopped.

In one embodiment, the subject is a transplant recipient undergoing immunosuppression therapy.

Suitably, the subject is a mammal. Suitably, the subject is a human.

The transplant may be selected from a liver, kidney, heart, lung, pancreas, intestine, stomach, bone marrow, vascularized composite tissue graft, and skin transplant.

Suitably, the cells may express a CAR which comprises an antigen binding domain which is capable of specifically binding to a HLA antigen that is present in the graft (transplant) donor but not in the graft (transplant) recipient.

Suitably, the transplant is a liver transplant. In embodiments where the transplant is a liver transplant, the antigen may be a HLA antigen present in the transplanted liver but not in the patient, a liver-specific antigen such as NTCP, or an antigen whose expression is up-regulated during rejection such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11.

As discussed above, in one representative and preferred embodiment the antigen is HLA-A2.

A method for treating a disease or condition relates to the therapeutic use of the cells herein. In this respect, the cells may be administered to a subject having an existing disease or condition in order to lessen, reduce or improve at least one symptom associated with the disease or condition and/or to slow down, reduce or block the progression of the disease.

Suitably, treating and/or preventing cellular and/or humoral transplant rejection may refer to administering an effective amount of the cells (e.g. Tregs) such that the amount of immunosuppressive drugs that a transplant recipient requires is reduced, or may enable the discontinuation of immunosuppressive drugs.

Preventing a disease or condition relates to the prophylactic use of the cells herein. In this respect, the cells may be administered to a subject who has not yet contracted or developed the disease or condition and/or who is not showing any symptoms of the disease or condition to prevent the disease or condition or to reduce or prevent development of at least one symptom associated with the disease or condition. The subject may have a predisposition for, or be thought to be at risk of developing, the disease or condition.

The autoimmune or allergic disease may be selected from inflammatory skin diseases including psoriasis and dermatitis (e.g. atopic dermatitis); responses associated with inflammatory bowel disease (such as Crohn's disease and ulcerative colitis); dermatitis; allergic conditions such as food allergy, eczema and asthma; rheumatoid arthritis; systemic lupus erythematosus (SLE) (including lupus nephritis, cutaneous lupus); diabetes mellitus (e.g. type 1 diabetes mellitus or insulin dependent diabetes mellitus); multiple sclerosis; neurodegenerative disease, for example, Amyotrophic Lateral Sclerosis (ALS); Chronic inflammatory demyelinating polyneuropathy (CIPD) and juvenile onset diabetes.

As indicated above, the recombinant protein is not limited to use in the context of immunosuppressive therapy, and the protein may be expressed in cells for the treatment of conditions such as cancer or infections. It may be desirable in such contexts to kill or ablate cancer or infected cells, and in such cases the chimeric protein may be expressed in cytotoxic cells, such as cytotoxic T cells or NK cells, or precursors therefor. The receptor (e.g. CAR or TCR) co-expressed with the chimeric protein in such cases may be directed against a cancer antigen or an antigen from a pathogen etc.

The medical use of or method herein may involve the steps of:
(i) isolating a cell-containing sample or providing a cell-containing sample;
(ii) introducing a nucleic acid molecule, construct or a vector as defined herein to the cell; and
(iii) administering the cells from (ii) to a subject.

The cell may be a Treg as defined herein. An enriched Treg population may be isolated and/or generated from the cell containing sample prior to, and/or after, step (ii) of the method. For example, isolation and/or generation may be performed prior to and/or after step (ii) to isolate and/or generate an enriched Treg sample. Enrichment may be performed after step (ii) to enrich for cells and/or Tregs comprising the recombinant protein, the nucleic acid molecule, construct, and/or the vector as described herein.

Suitably, the cell may be autologous. Suitably, the cell may be allogenic.

Suitably, the cell (e.g. the engineered Treg) may be administered is combination with one or more other therapeutic agents, such as lympho-depletive agents. The engineered cell, e.g. Treg, may be administered simultaneously with or sequentially with (i.e. prior to or after) the one or more other therapeutic agents.

Cells, e.g. Tregs, may be activated and/or expanded prior to, or after, the introduction of a nucleic acid molecule as described herein, for example by treatment with an anti-CD3 monoclonal antibody or both anti-CD3 and anti-CD28 monoclonal antibodies. Expansion protocols are discussed above.

The cell, e.g. Tregs, may be washed after each step of the method, in particular after expansion.

The population of engineered cells, e.g., Treg cells may be further enriched by any method known to those of skill in the art, for example by FACS or magnetic bead sorting.

The steps of the method of production may be performed in a closed and sterile cell culture system.

The invention may also provide a method for increasing the stability and/or suppressive function of a cell comprising the step of introducing a nucleic acid molecule, an expression construct or vector as provided herein into the cell. An increase in suppressive function can be measured as discussed above, for example by co-culturing activated antigen-specific Tconv cells with cells of the invention, and for example measuring the levels the cytokines produced by the Tconv cells. An increase in suppressive function may be an increase of at least 10, 20, 30, 40, 50, 60, 70, 80, or 90% as compared to a non-engineered Treg.

An increase in stability of a cell, e.g. a Treg as defined herein, refers to an increase in the persistence or survival of those cells or to an increase in the proportion of cells retaining a Treg phenotype over a time period (e.g. to cells retaining Treg markers such as FOXP3 and Helios) as compared to a non-engineered Treg. An increase in stability may be an increase in stability of at least 10, 20, 30, 40, 50, 60, 70, 80 or 90%, and may be measured by techniques known in the art, e.g. staining of Treg cell markers within a population of cells, and analysis by FACS.

A further aspect provided herein is a combination product comprising (a) a cell, cell population, vector or pharmaceutical composition as defined herein, and (b) EPO, for use in therapy, particularly ACT or gene therapy. The therapy may be any therapy as defined above, and further described herein.

The components (a) and (b) of the combination product may be for separate, sequential or simultaneous use.

The components (a) and (b) of the combination product will typically be provided as separate compositions, i.e. they will be formulated separately. Thus, the combination product may alternatively be defined or referred to as a kit.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto. All publications mentioned herein are incorporated herein by reference.

### EXAMPLES

### Example 1

### Materials and Methods

### Cloning:

Construct of the invention as shown in FIG. 2 were designed in house. Cloning may be carried out into the pMP71 backbone and D5a high efficiency bacteria may be transformed with plasmid and grown with the selection agent ampicillin. DNA may be extracted using a Miniprep Kit (Qiagen). Inserts can be transferred into a lentiviral backbone by PCR cloning.

### Collection of PBMCs:

Leukocyte cones can be supplied by NHS blood and transplant. PBMC may be isolated using a density centrifugation protocol. Briefly, blood may be diluted 1:1 with 1×PBS and layered over Ficoll-Paque (GE Healthcare). Samples may be centrifuged and the leukocyte layer removed and washed in PBS.

### Treg and Tconv Isolation Protocol:

Blood cones may be used to derive Treg and Teff populations. Blood cones may be subjected to CD4 enrichment via negative selection using RosetteSep™ Human CD4+ T Cell Enrichment Cocktail. Subsequently, CD4+ cells may be isolated using density centrifugation. CD4+ CD25+ T cells may then be isolated via positive selection using CD25 microbeads II (Miltenyi). The CD4+ CD25− fraction of cells may be retained to serve as conventional T cell (Tconv) populations. The CD4+CD25+ fraction may be stained with flow cytometry antibodies CD4 FITC (OKT4, Biolegend), CD25 PE-Cy7 (BC96, Biolegend), CD127 BV421 (A019D5, Biolegend), CD45RA BV510 (H1100, Biolegend) and the LIVE/DEAD™ Fixable Near-IR—Dead Cell Stain (Thermofisher) before FACS sorting. Where indicated, CD4+CD25+CD127low (Bulk Tregs) or CD4+CD25+ CD127low CD45RA+ (CD45RA+ Tregs) may be sorted and used.

### T Conv Culture Media:

Human Tconv may be grown in RPMI-1640 (Gibco) supplemented with 10% heat inactivated foetal bovine serum; penicillin; streptomycin; L-glutamine (Gibco).

### T Reg Culture Media and Expansion:

Human Regulatory T cells may be cultured in Texmacs media (Miltenyi) supplemented with IL-2 and activated with Human T-Activator CD3/CD28 Dynabeads™ (Gibco). Cells may be re-fed every 2 to 3 days with Treg culture media supplemented with IL-2. A second round of stimulation with Dynabeads™ may be performed to promote further expansion of Treg cells.

### Transfection and Viral Particle Production

HEK293T cells may be seeded and cultured in DMEM (Dulbecco's Modified Eagle's Medium)+10% Fetal Bovine Serum (FBS) for 24 hours. Transfection reagents may be brought to room temperature and mixed with DNA construct/plasmid of interest, packaging plasmid (pD8.91) and viral envelope (pVSV-G). PEI may be added to the diluted DNA and mixed and added to HEK293 Ts. Supernatant may be harvested 48 hours post-transfection, filtered and virus concentrated.

### Transduction of T Cells

Tconv may be activated with anti-CD3 and anti-CD28 Dynabeads (Gibco) and resuspended in T cell culture media. Non-tissue culture-treated 24-well plates may be prepared by coating with Retronectin (Takahara-bio—Otsu, Japan), cell suspension may be added together with lentiviral supernatant. Cells may be incubated and media exchange may be performed on alternate days. Cells may be used for experiments 7 days post-transduction.

### Flow Cytometry Staining

T cells may be removed from culture and washed in FACS buffer and stained with the LIVE/DEAD™ Fixable Near-IR—Dead Cell Stain (Thermofisher) in PBS first and then with anti-CD4 AF700 (RPA-T4, BD), and anti-CD3 PE-Cy7 in FACS staining buffer. For intracellular staining of FOXP3, cells may be fixed and permeabilized and stained with the anti-Foxp3 PE (150D/E4, Thermofisher) antibody. Cells may be analysed on a BD LSRII flow cytometer.

### Flow Cytometry Phenotyping of Transduced Cells

T cells may be removed from culture and stained for live cells using LIVE/DEAD™ Fixable Near-IR—Dead Cell Stain as described above. Surface staining of the cells may be performed using anti-CD4 AF700, anti-CD25 PE-Cy7 (BC96, Biolegend), anti-CD39 PerCPCy5.5 (A1, Biolegend), anti-CD62L PE-CF594 (DREG-56, BD), anti-TIM3 BV786 (7D3, BD), anti-TIGIT BV605 (A15153G, Biolegend), anti-CD45RO BUV395 (UCHL1, BD), anti-CD279 BUV737 (EH12.1, BD) and anti-CD223 BV711 (11C3C65, Biolegend). Cells may be permeabilised and stained with anti-Foxp3 PE (150D/E4, Thermofisher).

### Data Analysis

Flow cytometric data maybe analysed using the Flow Cytometry Analysis software FlowJo (Flowjo, LLC). All statistical Analysis maybe performed using Graphpad Prism v.5 (Graphpad, Software)

### Example 1a: Expression in Jurkats

Constructs may be cloned into a lentiviral backbone encoding a puromycin resistance gene, as described above. Viral vectors maybe produced and used for the transduction of the Jurkat T cell line. Two days after transduction, Jurkat cells can be selected with 4 μg/ml puromycin for one week. Cells may be counted and 0.5*10⁶ cells may be stained with an anti-EPOR antibody to determine the level of recombinant protein expressed in the cells.

Expression may be assessed by flow cytometry as described above.

### Example 2: STAT5 Signaling of Constructs

Different constructs of the recombinant protein are cloned into a lentiviral backbone encoding a puromycin resistance gene. Viral vectors are produced and used for the transduction of a NFAT, NfkB and STAT5 Jurkat reporter cell line. Here, the NFAT, NfkB or STAT5 response element control the activity of a luc2 reporter gene. Two days after transduction, Jurkat cells are selected with 4 μg/ml puromycin for one week. Luciferase is assessed in the different reporter cell lines using ONE-Glo™ Luciferase Assay System (Promega).

### Example 3: Generation of Regulatory T Cells Expressing the Chimeric Protein

Regulatory T cells are purified and FACS sorted as CD4+ CD25+ CD127− cells from healthy donors. Cells are activated using Human T-Activator CD3/CD28 Dynabeads™ (ThermoFisher Scientific) in X-Vivo medium (Lonza) in the presence of Interleukin-2 (1000 IU/ml). After 48 hours of activation, cells are transduced with lentiviral particles, encoding the recombinant protein constructs. Cells are further expanded, and expansion rate is compared between the different conditions. At day 14 cells are harvested and counted. 0.5*106 cells are stained with an anti-EPOR antibody. The level of EPOR expression and transduction efficiency was assessed by Flow Cytometry looking at the percentage of anti-EPOR antibody. The Treg phenotype is assessed by surface staining with anti-CD4, anti-CD25, anti-CD127, anti-CD8, anti-GITR, anti-CD39, anti-CD45RA, anti-CD45RO, anti-ICOS and intracellular staining with anti-FOXP3 and anti-HELIOS, following fixation and permeabilization (Transcription Factor Staining Buffer Set, ThermoFisher Scientific) Example 4: STAT5 phosphorylation analysis as an indicator of IL2R signalling Transduced Tregs with chimeric protein were rested overnight in culture media without IL2. STAT5 phosphorylation of Tregs was assessed by FACS analysis 10 and 120 minutes after culture with media alone, or rapamycin.

### Example 5: Analysis of Constructs 783, 784, 785, 786, 787 and 788

### Materials and Methods

### Cloning:

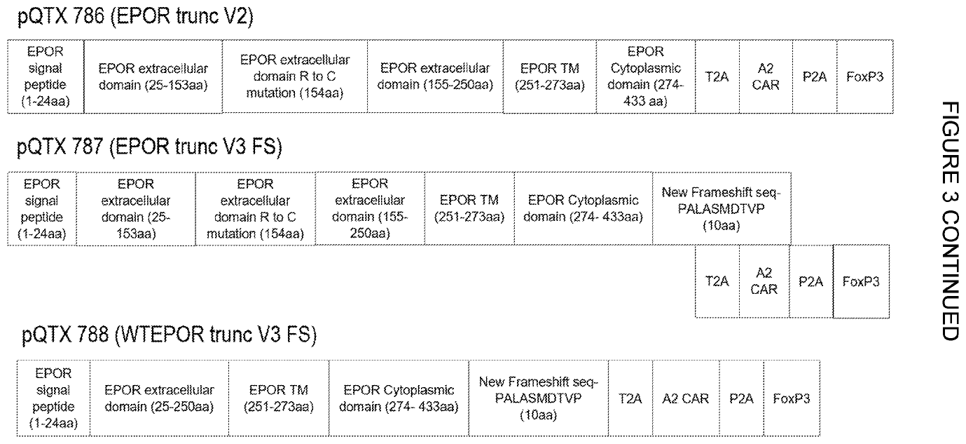

The constructs shown in FIG. 3 were designed in house. Cloning was carried out in a lentiviral vector backbone and NEB Stable high efficiency bacteria was transformed with plasmid and grown with the selection agent kanamycin. DNA was extracted using a Miniprep Kit and Maxiprep Kits (Qiagen). Inserts were transferred into other lentiviral or retroviral backbones by PCR cloning. Cloning was carried out by site-directed mutagenesis or PCR-based methods.

### Constructs:

Construct pQTX 783 (EPOR WT) comprises a wild-type EPO receptor operatively linked to a CAR specific for HLA-A2 and FoxP3 (separated by T2A and P2A self-cleaving domains).

Construct pQTX 784 (EPOR mut) comprises a mutated EPO receptor operatively linked to a CAR specific for HLA-A2 and FoxP3 (separated by T2A and P2A self-cleaving domains). The mutated EPOR has a mutation in the exodomain (R is mutated to C at position 154 of SEQ ID NO. 1). The transmembrane domain and endodomain of the mutated EPOR are wild type EPOR transmembrane and endodomains respectively.

Construct pQTX 785 (EPOR trunc V1) comprises a mutated EPO receptor operatively linked to a CAR specific for HLA-A2 and FoxP3 (separated by T2A and P2A self-cleaving domains). The mutated EPOR has a mutation in the exodomain (R is mutated to C at position 154 of SEQ ID NO. 1) and the endodomain has been truncated after position 378 of SEQ ID NO. 1. The transmembrane domain is the wild type EPOR transmembrane domain.

Construct pQTX 786 (EPOR trunc V2) comprises a mutated EPO receptor operatively linked to a CAR specific for HLA-A2 and FoxP3 (separated by T2A and P2A self-cleaving domains). The mutated EPOR has a mutation in the exodomain (R is mutated to C at position 154 of SEQ ID NO. 1) and the endodomain has been truncated after position 433 of SEQ ID NO. 1. The transmembrane domain is the wild type EPOR transmembrane domain.

Construct pQTX 787 (EPOR trunc V3 FS) comprises a mutated EPO receptor operatively linked to a CAR specific for HLA-A2 and FoxP3 (separated by T2A and P2A self-cleaving domains). The mutated EPOR has a mutation in the exodomain (R is mutated to C at position 154 of SEQ ID NO. 1), the endodomain has been truncated after position 433 of SEQ ID NO. 1 and an additional 10 amino acid tail has been inserted at the C-terminus (PALASMDTVP). The transmembrane domain is the wild type EPOR transmembrane domain.

Construct pQTX 788 (WT EPOR trunc V3 FS) comprises a mutated EPO receptor operatively linked to a CAR specific for HLA-A2 and FoxP3 (separated by T2A and P2A self-cleaving domains). The exodomain and transmembrane domains are the wild type EPOR exodomain and transmembrane domain respectively. The endodomain has been truncated after position 433 of SEQ ID NO. 1 and an additional 10 amino acid tail has been inserted at the C-terminus (PALASMDTVP).

### Collection of PBMCs:

Leukopaks were supplied by BiolVT. PBMCs were isolated using a negative selection kit (StemCell Technologies). Briefly, unwanted fractions are labelled and targeted for removal with antibody complexes and magnetic beads and subsequently separated using a magnet.

### Treg and Tconv (Teffs) Isolation Protocol:

Leukopaks were used to derive Treg and Tconv populations. Leukopak-derived PBMCs were subjected to CD25 positive selection followed by a CD4 enrichment via negative selection using Human CD25 Positive Selection Cocktail and Human CD4+ T Cell Enrichment Cocktail (Stemcell Technologies), respectively. Cells of interest were separated using a magnet. The CD4+ CD25− fraction of cells were retained to serve as Tconv populations. CD127high Depletion Cocktail (Stemcell Technologies) were used to further separate the CD4+CD25+CD127−/low cell populations and cells of interest separated using a magnet. Cell fractions were stained with flow cytometry antibodies anti-CD4 VioBlue (M-T466, Miltenyi Biotec), anti-CD25 PE (3G10, Miltenyi Biotec), anti-CD127 APC (MB15-18C9, Miltenyi Biotec), anti-CD45RA FITC (T6D11, Mitenyi Biotec) and LD 7AAD (BioLegend) before FACS sorting. Where indicated, CD4+CD25+CD127−/low (Bulk Tregs) or CD4+CD25+CD127−/low CD45RA+ (CD45RA+ Tregs) were sorted and used.

### Treg Culture Media and Expansion:

Human Regulatory T cells were cultured in X-VIVO 15 media (Lonza) supplemented with 5% Human AB serum (heat-inactivated; Merck), IL-2 (Proleukin, Clinigen Healthcare) and activated with anti-CD3/CD28 beads. Cells were re-fed every 2 to 3 days with Treg culture media supplemented with IL-2. A second round of stimulation with anti-CD3/CD28 beads was performed to promote further expansion of Treg cells.

### Transfection and Viral Particle Production:

HEK293T cells were seeded and cultured in DMEM (Dulbecco's Modified Eagle's Medium)+10% Fetal Bovine Serum (FBS) for 24 hours. Transfection reagents were brought to room temperature and mixed with DNA construct/plasmid of interest, packaging plasmids (pMDLg pRRE), regulatory plasmid (PRSV-REV) and viral envelope (pMD2.G). FuGENE HD Transfection reagent (Promega) were added to the diluted DNA and mixed and added to HEK293 Ts. Supernatant was harvested 48 hours post-transfection, filtered and virus concentrated.

### Transduction of T Cells:

Tregs were activated with anti-CD3/CD28 beads and resuspended in T cell culture media (X-Vivo 15 medium (Lonza), 5% Human AB serum and IL2). Non-tissue culture-treated 24-well plates were prepared by coating with Retronectin (Takahara-bio—Otsu, Japan), cell suspension was added together with lentiviral supernatant. Cells cultures were spinoculated and media exchange performed on alternate days. Cells were used for experiments 7 days or more post-transduction.

### Transduction Efficiency:

Tregs were first surface stained with HLA-A2 Dextramer-APC (Immudex) for 15 minutes at room temperature. Washed cells were then LIVE/DEAD™ Fixable Near-IR—Dead Cell Stain (Thermofisher) in PBS for 20 minutes at room temperature. Washed cells were then stained with anti-CD4 BV510 (A161A1; BioLegend), anti-CD25 PE-Cy7 (BC96; BioLegend) and anti-EPOR PE (38409; Bio-Techne) in FACS staining buffer at 4 degrees C. for 20 minutes. Cells were washed before acquisition on the Attune™ NxT flow cytometer. Cell surface expression was analysed using FlowJo software. The following gating strategy was used: Lymphocytes>single cells>viable cells>CD4+CD25+>HLA-A2 Dex+ EPOR+.

pSTAT5 Assay:

Fourteen days post Treg isolation, transduced Tregs were rested by depletion of anti-CD3/CD28 beads and removal of IL2 for 24-48 hours prior to assay set up. Tregs were treated with or without 1 IU/ml EPO (Stemcell Technologies UK) for 30 mins at 37 degrees C. before commencing staining. Tregs were first surface stained with HLA-A2 Dextramer-APC (Immudex) for 15 minutes at room temperature. Washed cells were then stained with LIVE/DEAD™ Fixable Near-IR—Dead Cell Stain (Thermofisher) in PBS for 20 minutes at room temperature. Washed cells were then stained with anti-CD4 BV510 (A161A1; BioLegend) and anti-CD25 PE-Cy7 (BC96; BioLegend) in FACS staining buffer at 4 degrees C. for 20 minutes. Cells were then fixed and permeabilised using the PerFix Expose kit (Beckman Coulter) before intracellular staining with pSTAT5 PE-CF594 (47/Stat5 pY694; BD Bioscience). Cells were washed twice before acquisition on the Attune™ NxT flow cytometer. pSTAT5 expression was analysed using FlowJo software. The following gating strategy was used: Lymphocytes>single cells>viable cells>CD4+CD25+>HLA-A2 Dex+>pSTAT5+. Except, for Mock Tregs, the following gating strategy was used: Lymphocytes>single cells>viable cells>CD4+CD25+>pSTAT5+.

### Measuring Transduced Cell Enrichment and FOXP3 Expression:

Fourteen days post Treg isolation, transduced Tregs were rested by depletion of anti-CD3/CD28 beads and removal of IL2 for 24 hours prior to assay set up. Various conditions consisting of media, CD3/CD28 beads, 0 and 300 IU/ml IL2 were set up for days 0, 4 and 6 readouts. Tregs were first surface stained with HLA-A2 Dextramer-APC (Immudex), then LIVE/DEAD™ Fixable Near-IR—Dead Cell Stain (Thermofisher) in PBS. Washed cells were then stained with anti-CD4 BV510 (A161A1; BioLegend) and anti-CD25 PE-Cy7 (BC96; BioLegend) in FACS staining buffer for 30 minutes at 4° C. Washed cells were then permeabilised using FOXP3/Transcription Factor Staining Buffer Set (Thermo Fisher Scientific, UK) according to the manufacturer's instructions. Permeabilised cells were stained with anti-FoxP3 BV421 (206D; BioLegend) antibody. Cells were washed twice before acquisition on the Attune™ NxT flow cytometer. FoxP3 expression was analysed using FlowJo software. The following gating strategy was used: Lymphocytes>single cells>viable cells>CD4+CD25+>HLA-A2 Dex+>FoxP3+. Except, for Mock Tregs, the following gating strategy was used: Lymphocytes>single cells>viable cells>CD4+CD25+>FoxP3+.

### Measuring Cell Viability/Fold Expansion:

Fourteen days post Treg isolation, transduced Tregs were rested by depletion of anti-CD3/CD28 beads and removal of IL2 for 24 hours prior to assay set up. Various conditions consisting of media, A1+K562 (which were A2−) or A2+K562 cells in the absence of IL2 were set up for days 0, 5 and 7 readouts. A ratio of 1:4 of K562:Tregs were used. Tregs were surface stained with anti-EPOR PE (38409; Bio-Techne), anti-CD34 FITC (QBEND10; Life Technologies), anti-CD4 BV510 (A161A1; BioLegend) and anti-CD25 PE-Cy7 (BC96; BioLegend). Washed cells were then subsequently stained with Annexin V BV421 and 7-AAD in Annexin V Binding Buffer using Annexin V Apoptosis Detection Kit with 7-AAD (BioLegend). Equal volumes of cell suspension were acquired across all wells to allow for fair quantification of viable transduced cells in each well. Cells were acquired on the Attune™ NxT flow cytometer. Cell viability was analysed using FlowJo software. The following gating strategy was used: Lymphocytes>single cells>Annexin V-7-AAD- (viable cells)>CD4+CD25+>CD34/QBEND+ or EPOR+.

### Deep Phenotyping:

Fourteen days post Treg isolation, transduced Tregs were rested by depletion of anti-CD3/CD28 beads and removal of IL2 for 24 hours prior to staining. Tregs were first surface stained with HLA-A2 DextramerAPC (Immudex) for 15 minutes at room temperature. Washed cells were then stained with LIVE/DEAD™ Fixable eFluor455—Dead Cell Stain (Thermofisher) in PBS for 20 minutes at room temperature. Washed cells were then stained with anti-CCR4 BV480 (1G1; BD Biosciences), anti-CCR7 BUV395 (2-L1-A; BD Biosciences), anti-CD183/CXCR3 PE-Cy7 (G025H7; Biolegend) in Brilliant Stain Buffer Plus at 37 degrees C. for 20 mins. Washed cells were then stained with anti-HLA-DR APC-Fire810 (G46-6; BD Bioscience), anti-PD-1 BV650 (EH12.2H7; Biolegend), anti-ICOS BV750 (DX29; BD Biosciences), anti-CD8a BUV805 (SK1; BD Biosciences), anti-TIM-3/CD366 BV711 (7D3; BD Biosciences), anti-CD27 PE-AF700 (0323; Biolegend), anti-CCR6 BV605 (G034E3; Biolegend), anti-CD95 PE-Cy5 (DX2; BD Biosciences), anti-TIGIT APC-Cy7 (VSTM3; Biolegend), anti-CD70 BV786 (Ki-24; BD Biosciences), anti-CD4 VioBlue (M-T466; Miltenyi Biotec), anti-CD34 PE (QBEND10; Life Technologies) and anti-CD25 BUV563 (2A3; BD Biosciences) in Brilliant Stain Buffer Plus at 4 degrees C. for 30 mins. For intracellular staining of cells, cells were fixed and permeabilized using FOXP3/Transcription Factor Staining Buffer Set (Thermo Fisher Scientific, UK) according to the manufacturer's instructions. Permeabilised cells were stained with the anti-Helios PE-Dazzle594 (22F6; Biolegend), anti-CTLA-4 BB700 (BNI3; BD Biosciences) and anti-FOXP3 AF647 (206D; BD Biosciences) in permeabilization buffer. Cells were washed twice before acquisition on a flow cytometer. Marker expression was analysed using FlowJo software.

### Suppression Assay:

Fourteen days post Treg isolation, transduced Tregs were rested by depletion of anti-CD3/CD28 beads and removal of IL2 for 24 hours prior to assay set up. CellTrace™ Violet (CTV) Cell Proliferation Kit (ThermoFisher) was used to stain Tregs and CellTrace™ Yellow (CTY) Cell Proliferation Kit (ThermoFisher) was used to stain T-effectors according to the manufacturer's protocols. CTV stained Tregs were cultured with CTY-stained T-effectors at the indicated different ratios. 120 Gy irradiated HLA-A2+ B cells or HLA-A2− B cells were co-cultured at a 1:3.3 of B cells: T-effectors ratio. Alternatively, Human T-Activator CD3/CD28 Dynabeads™ (Gibco) were added at a 1:1 beads: T-effectors ratio. Cell cultures were incubated at 37° C. at 5% CO₂ for 4 days prior to staining. Cells were washed in PBS prior to staining with Fixable Near IF—Dead Cell Stain (Thermofisher) in PBS for 20 minutes at room temperature. Washed cells were then stained with anti-EPOR PE (38409; Bio-Techne), anti-CD34 FITC (QBEND10; Life Technologies) and HLA-A3 (REA950; Miltenyi Biotec) for 20 minutes at 4° C. Cells were washed before acquisition on the Attune™ NxT flow cytometer. Data was analysed using FlowJo software. The following gating strategy: Lymphocytes>single cells>viable cells>HLA-A3->CTY+.

### Cytotoxicity Assay:

Target cells of HLA-A2+ or HLA-A2− K562 were labelled with CellTrace™ Violet (CTV) Cell Proliferation Kit (ThermoFisher) according to the manufacturer's protocols. Effector cells of indicated Tregs or Natural Killer cells (NK) were co-cultured with the target cells at the various indicated ratios. Anti-CD107a PE (H4A3; BD Biosciences) was added to the cultures and incubated for a total of 4 hours at 37° C. and 5% CO₂. After 1 hour of incubation, GolgiSTOP working solution (BD Biosciences) was added to the cultures. Washed cells were acquired on the Attune™ NxT flow cytometer. Data was analysed using FlowJo software.

### Intracellular Cytokine Staining:

Tregs or T-cells were activated with Leukocyte Activation Cocktail with BD GolgiPlug™ for 5 hours at 37° C. Cells were then stained with LIVE/DEAD™ Aqua in PBS for 20 minutes at room temperature. Washed cells were then stained with anti-CD4 PerCPCy5.5 (SK3; Biolegend), anti-EPOR PE (38409; Bio-Techne) and anti-CD34 FITC (QBEND10; Life Technologies) for 30 minutes at 4° C. Washed cells were then fixed and permeabilised using FOXP3/Transcription Factor Staining Buffer Set (Thermo Fisher Scientific, UK) according to the manufacturer's instructions. Permeabilised cells were then stained with anti-IL-17A BV605 (BL168: Biolgend), anti-TNFα APC-Cy7 (Mab11; Biolegend), and anti-IFN-γ PE-Cy7 (4S.B3; Biolgend) and anti IL2 PE-PE/Dazzle 594 (MQ1-17H12; Biolegend). Cells were washed before acquisition on a flow cytometer.

### Data Analysis:

Flow cytometric data maybe analysed using the Flow Cytometry Analysis software FlowJo (FlowJo, LLC). All statistical Analysis maybe performed using Graphpad Prism v.9.4.1 (Graphpad Prism Software)

### Results

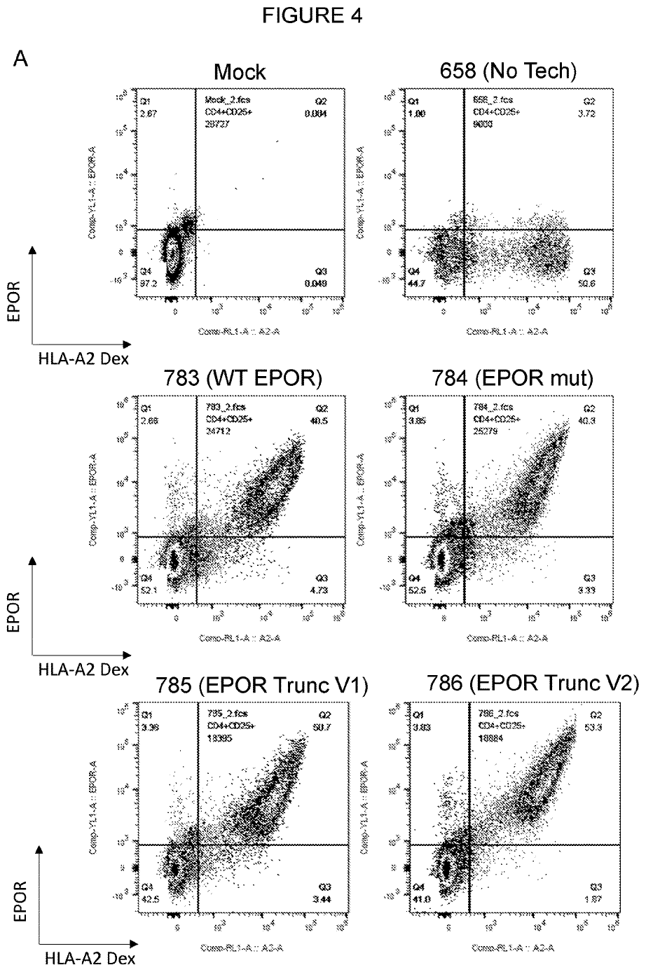

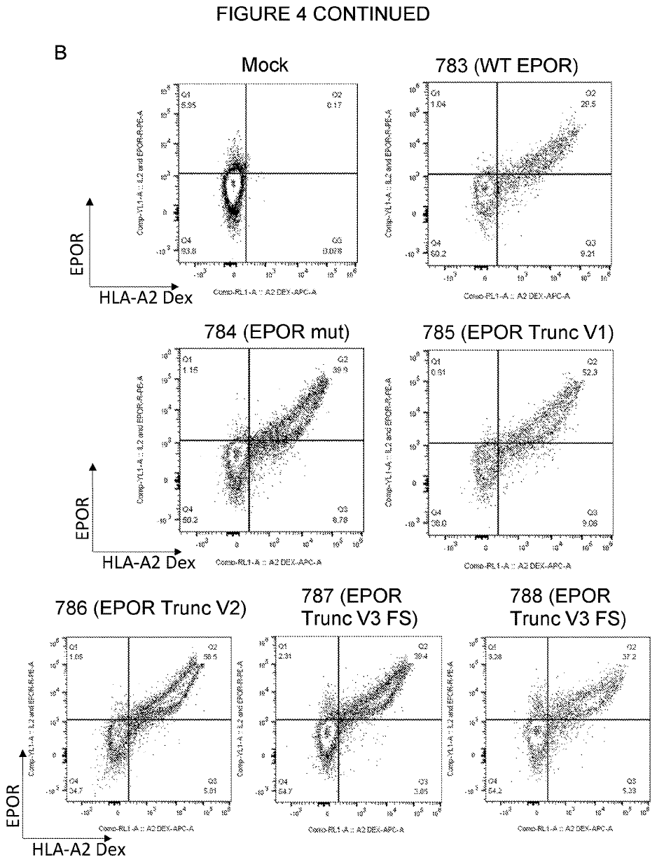

### FIG. 4A—Constructs are Well Expressed in Transduced Human Tregs

FACS plots showing the transduction efficiencies of fresh Tregs transduced with the various constructs of 658 (no tech), 783, 784, 785, 786 and Mock are also shown. Transduction efficiencies were measured using an HLA-A2 dextramer and EPOR expression. A similar transduction efficiency was obtained for each construct. This is also shown in the table below.

| Construct | Transduction efficiency (%) |
|---|---|
| 658 | 50.6 |
| 783 | 40.5 |
| 784 | 40.3 |
| 785 | 50.7 |
| 786 | 53.3 |

### FIG. 4B—Constructs are Well Expressed in Transduced Human Tregs

FACS plots showing the transduction efficiencies of frozen Tregs transduced with the various constructs of 783, 784, 785, 786, 787, 788 and Mock are also shown. Transduction efficiencies were measured using an HLA-A2 dextramer and EPOR expression. A similar transduction efficiency was obtained for each construct. This is also shown in the table below.

| Construct | Transduction efficiency (%) |
|---|---|
| 783 | 29.5 |
| 784 | 39.9 |
| 785 | 52.3 |
| 786 | 58.5 |
| 787 | 39.4 |
| 788 | 37.2 |

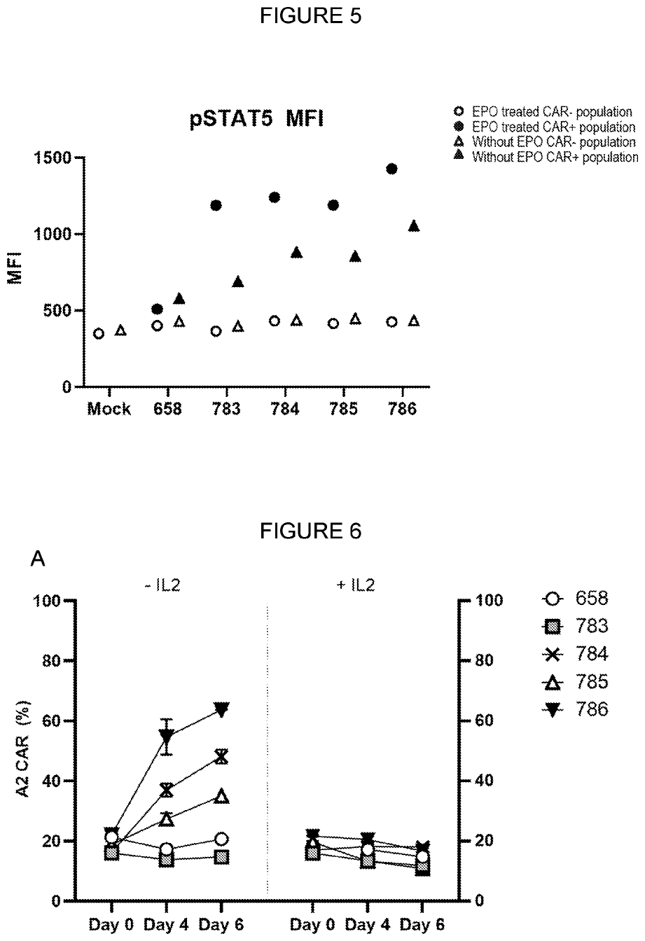

FIG. 5—Tregs Transduced with EPOR-Derived Proteins Specifically Upregulate pSTAT5 Levels

Transduced Tregs were treated with or without EPO before staining for pSTAT5 expression levels. Cells were pre-gated on the transduced fraction defined as HLA-A2 CAR+, whereas the untransduced fractions are HLA-A2 CAR−. The transduced fraction of cells expressing one of constructs 783, 784, 785 and 786 had higher MFI levels of pSTAT5 compared to the untransduced fraction of cells indicating that the EPOR-derived proteins are promoting JAK-STAT signalling. The transduced fraction of cells expressing one of constructs 783, 784, 785 and 786 also specifically responded to exogenous EPO as detected by higher MFI levels of pSTAT5 whereas the untransduced fractions did not respond to exogenous EPO and this untransduced fraction remained at similar basal MFI levels of pSTAT5. Cells transduced with construct 658, which does not have an EPOR-derived protein, had similar MFI levels of pSTAT5 in both the transduced and untransduced fractions. It is noted that the media used (X-vivo plus 5% human serum) likely contains EPO as EPO is found in serum. This explains the increase in pSTAT5 for cells transduced with construct 783 (WT EPOR) and further increase when more EPO is added. However, where additional EPO is not used, the transduced fraction for 784, 785 and 786 have higher pSTAT5 than 783.

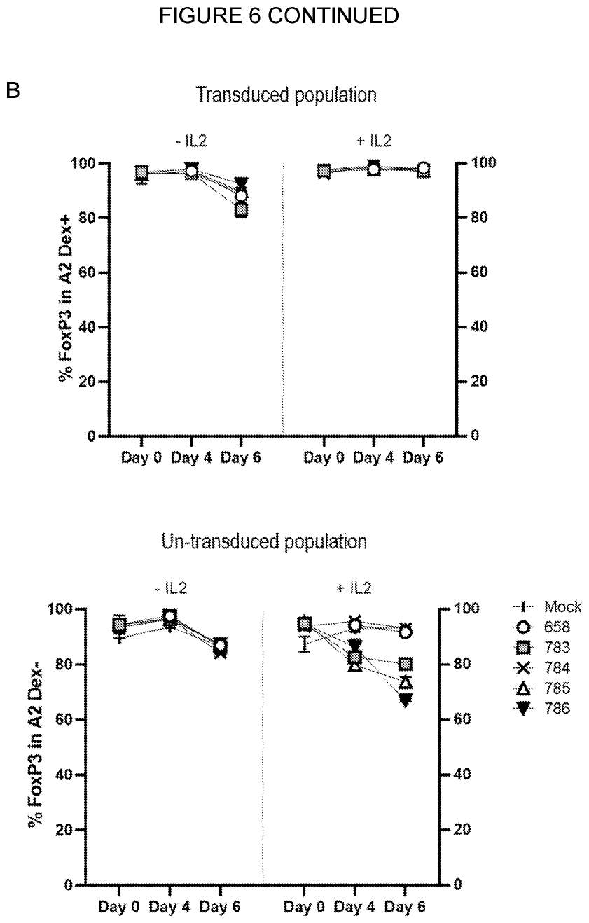

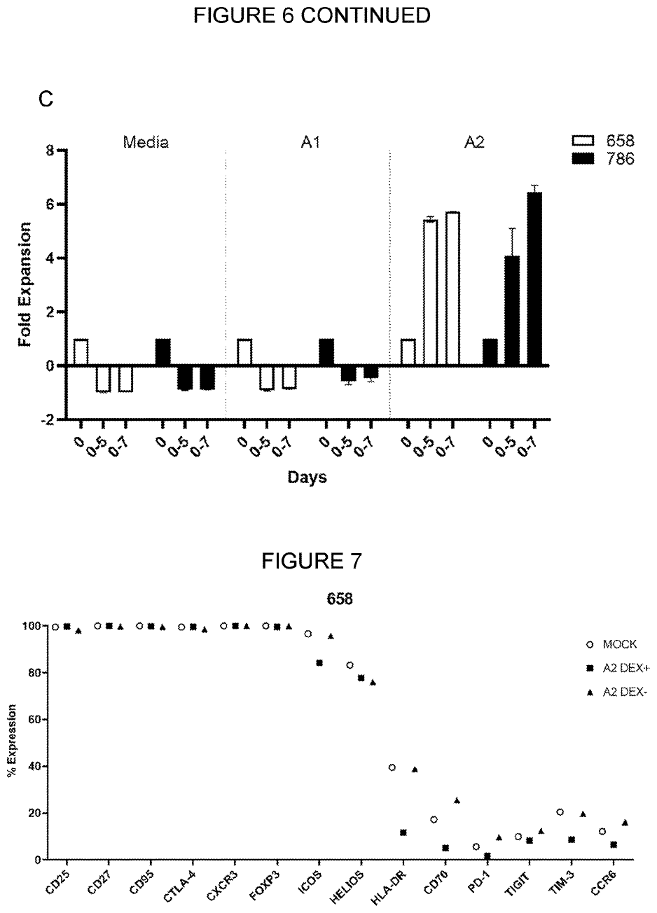

FIG. 6A—Tregs Transduced with EPOR-Derived Proteins Enrich Over Time and have a Better Survival Advantage Compared to Control Tregs

Transduced Tregs were cultured with or without IL2. Cells transduced with the constitutive EPOR technology of 784, 785 and 786 show enrichment of the percentage of population over time. In contrast, the 658 (no EPOR-derived protein) and 783 (WT EPOR) constructs do not preferentially enrich over time. The cells transduced with the constitutive EPOR (784, 785 and 786) display a survival advantage over the 783 (WT EPOR) and 658 (no tech) when IL2 is not present. The 786 (EPORtrunc V2) appears to have the highest advantage as these transduced cells see an increase in their percentage over time compared to un-transduced. When IL2 is present, the untransduced fraction of cells can expand as a result of signaling through natural IL2R and therefore the population of cells transduced with the constitutive EPOR technology does not enrich.

FIG. 6B—Tregs Transduced with EPOR-Derived Protein Maintain FoxP3 Expression Over Time

Transduced Tregs were cultured with or without IL2. The percentage of cells expressing FoxP3 were similar across the transduced fractions of 783, 784, 785 and 786 compared to 658. Within the untransduced fraction of cells, the percentage of FoxP3 expression were slightly lower than the transduced fraction of cells. Cells transduced with the EPOR-derived proteins expressed and maintained the FoxP3 expression levels over time indicating that the Tregs maintain their phenotype.

### FIG. 6C—786 Transduced Tregs Recognise Cognate A2 Antigen and Respond in a Similar Manner to 658 Control Tregs

Survival assay showing with 658 and 786 transduced Tregs co-cultured with A2+ K562 or A2− K562 (A1+ K562) cells. The data shows that both 658 and 786 transduced Tregs recognise and respond specifically to A2 antigen by cell expansion fold changes. Thus, transduction with the constitutive EPOR technology (786) does not negatively affect the function of the cells.

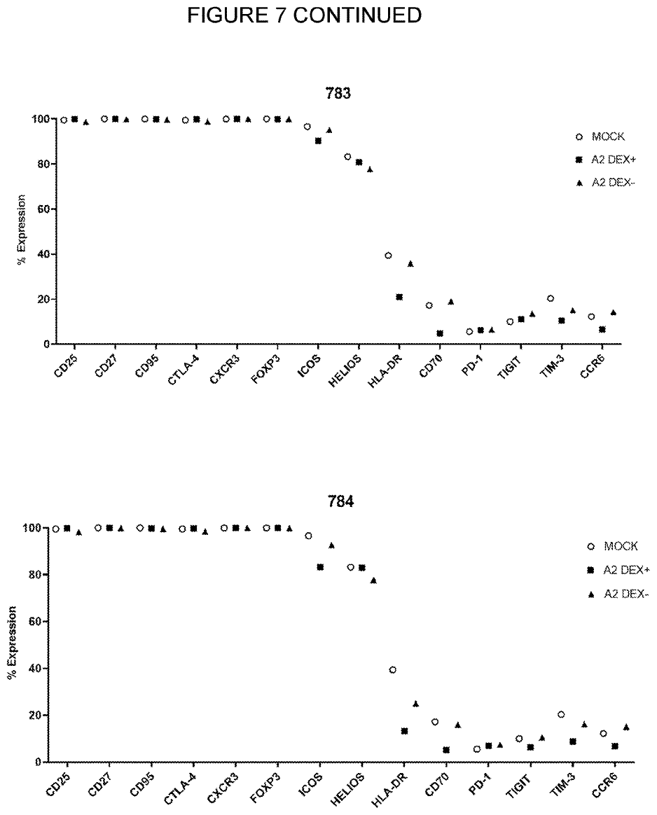

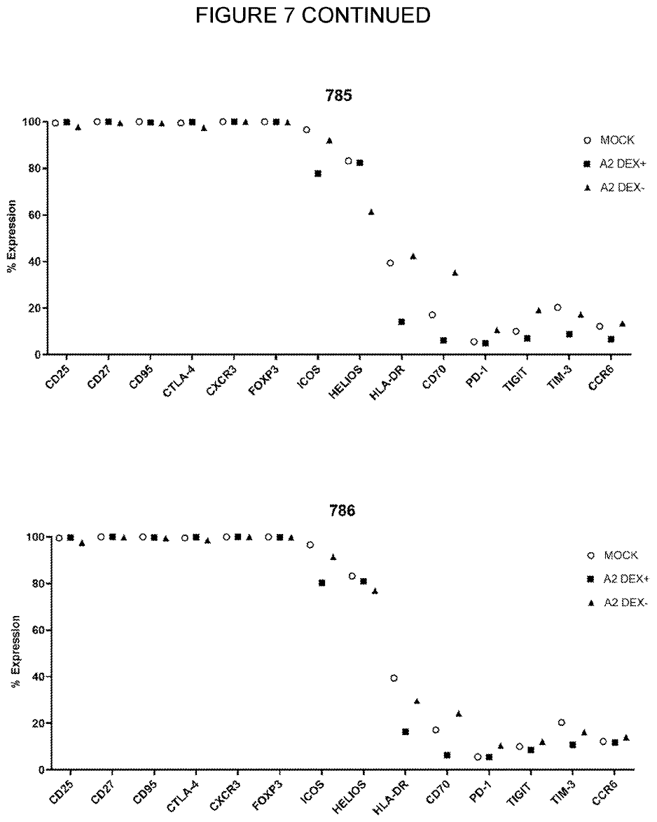

FIG. 7—Tregs Transduced with EPOR-Derived Proteins Maintained Key Treg Marker Expression

Transduced Tregs were deep phenotyped for various Treg associated markers. The graph shows the percentage of expression of the indicated marker within the Mock Tregs, transduced fraction (HLA-A2 Dex+) or untransduced fraction (HLA-A2 DEX−) of cells. EPOR-transduced Tregs (both transduced and untransduced fractions) maintained high and similar expression levels of key Treg markers such as CD25, CD27, CD95, CTLA-4, CXCR3 and FoxP3 compared to Mock transduced Tregs. The results suggest that transduction with the EPOR-derived proteins did not have a significant effect on the phenotype of the Tregs.

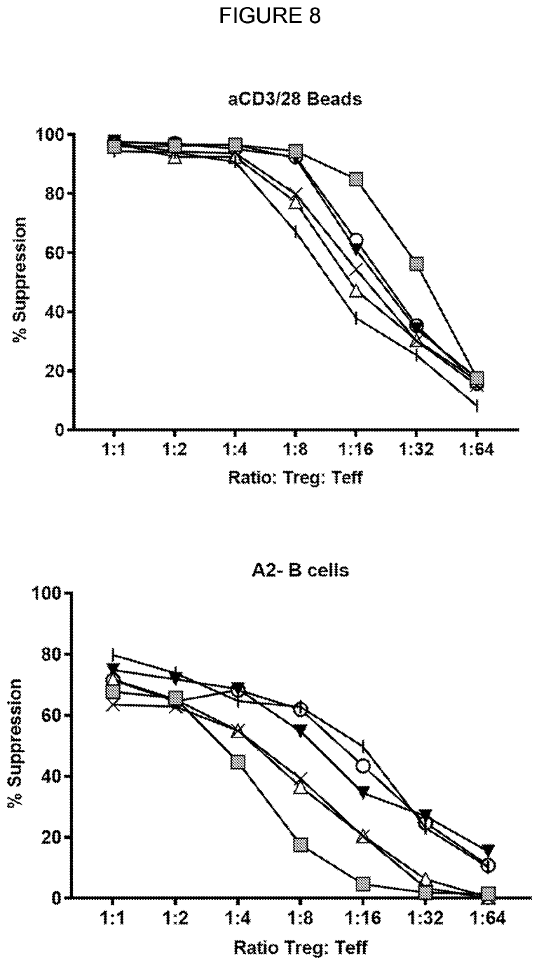

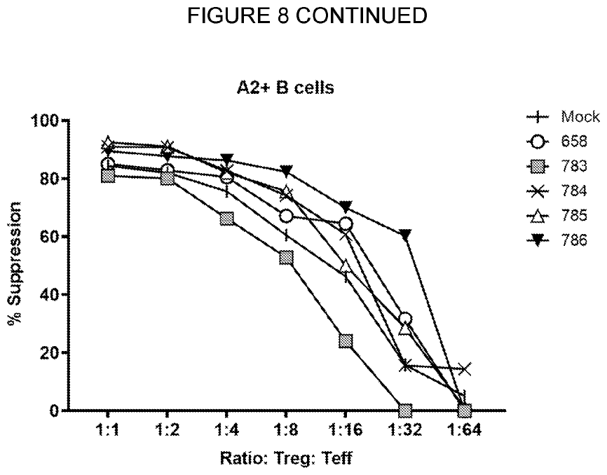

FIG. 8—Tregs Transduced with EPOR-Derived Proteins Maintained Suppressive Ability

Transduced Tregs were functionally tested for suppressive capacity. The graph shows the percentage of suppression of transduced Tregs (or Mock Tregs) against the proliferation of T-effector cells with the various stimulus of aCD3/28 beads, HLA-A2− B cells and HLA-A2+ B cells. The results indicate that the Tregs with the EPOR technology has a similar suppressive ability as Mock Tregs.

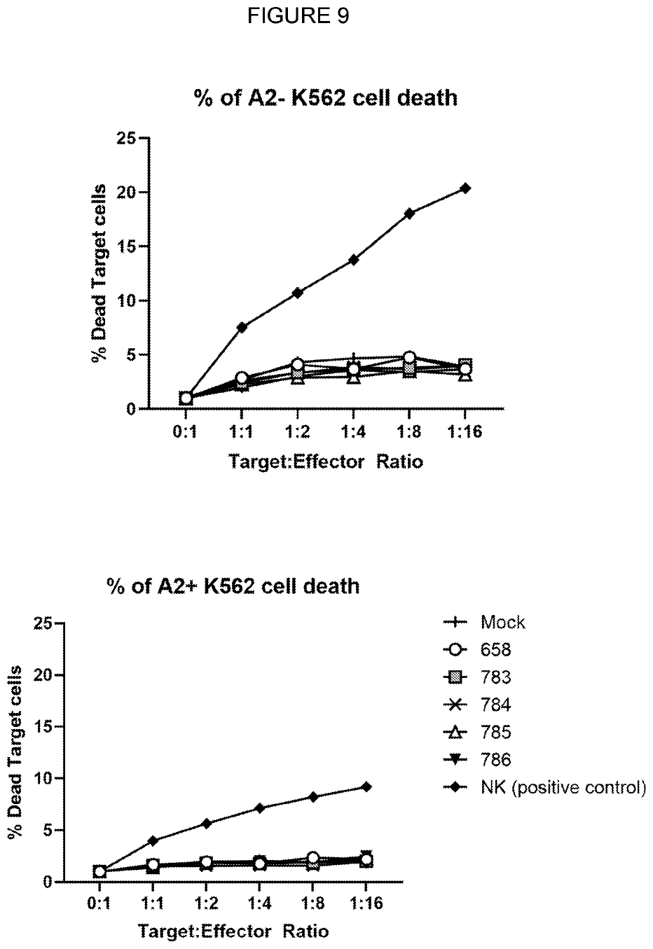

FIG. 9—Tregs Transduced with EPOR-Derived Proteins do not Kill Target Cells

Transduced Tregs were tested for cytotoxicity towards target cells. HLA-A2− or HLA-A2+ K562 cells were co-cultured with transduced Tregs at various ratios. As a positive control HLA-A2+ Natural Killer (NK) cells were used. The results indicate that Mock and EPOR transduced Tregs do not kill the target cells or non-target cells and thus the regulatory phenotype is maintained.

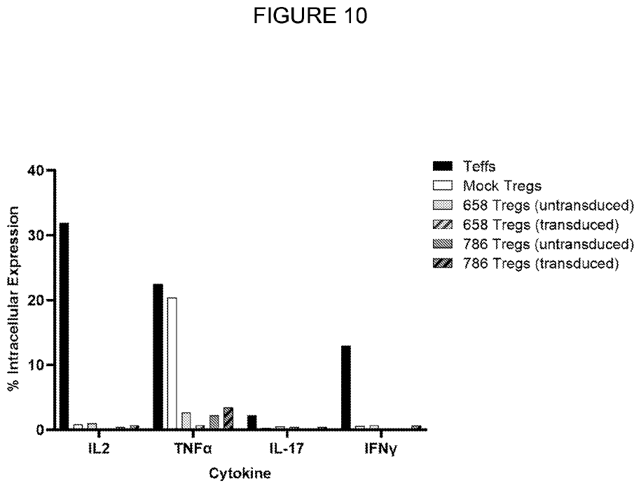

FIG. 10—Tregs Transduced with EPOR-Derived Proteins have a Similar Intracellular Cytokine Profile to Control Tregs

658 and 786 Transduced Tregs were tested for intracellular cytokine presence. Cells were stimulated with PMA/ionomycin before detecting intracellular cytokines. T-effector cells were used as a positive control. 786 transduced Tregs, both transduced and untransduced fractions had similar and low levels of intracellular cytokines IL2, TNFα IL-17 and IFNγ to 658 transduced Tregs. This suggests that the 786 construct does not cause an inflammatory response in the cells and the cells maintain their regulatory phenotype.

1. A T cell comprising a recombinant protein, wherein said recombinant protein comprises an exodomain, at least a portion of which is derived from the extracellular region of EPOR, wherein the exodomain comprises a dimerisation domain allowing dimerisation of said recombinant protein with a second protein and provision of a signal into said T cell in the absence of a signal inducer molecule.

2. The T cell of claim 1, wherein said at least a portion of said exodomain comprises an amino acid sequence having at least 70% sequence identity to SEQ ID NO. 3 or 5.

3. The T cell of claim 1 or 2, wherein said at least a portion of said exodomain derived from the extracellular region of EPOR comprises said dimerisation domain.

4. The T cell of any one of claims 1 to 3, wherein said dimerisation domain is a leucine zipper or a cysteine residue.

5. The T cell of any one of claims 1 to 4, wherein said dimerisation is disulphide-linked dimerisation.

6. The T cell of any one of claims 1 to 5, wherein said dimerisation is homodimerization or heterodimerisation.

7. The T cell of any one of claims 1 to 6, wherein said signal inducer molecule is EPO.

8. The T cell of any one of claims 1 to 7, wherein said exodomain comprises or consists of an amino acid sequence as set out in SEQ ID NO. 2 or a variant thereof having at least 80% sequence identity thereto and having a cysteine residue at position 130, or comprises or consists of an amino acid sequence as set out in SEQ ID NO. 4 or variant thereof having at least 80% sequence identity thereto and having a cysteine residue at position 154.

9. The T cell of any one of claims 1 to 8, wherein said T cell is a T regulatory cell, or a precursor thereof.

10. The T cell of any one of claims 1 to 9, wherein said second protein is different to said recombinant protein.

11. The T cell of claim 10, wherein said T cell further comprises said second protein.

12. The T cell of any one of claims 1 to 11, wherein said T cell further comprises a chimeric antigen receptor, a heterologous TCR, a safety switch polypeptide, a heterologous FOXP3 polypeptide and/or a mutated calcineurin protein.

13. The T cell of any one of claims 1 to 12, wherein said exodomain comprises one or more domains or sequence(s) heterologous to EPOR.

14. The T cell of claim 13, wherein said exodomain comprises one or more tag peptides, suicide moieties and/or inducible dimerisation domains.

15. The T cell of any one of claims 1 to 14, wherein said extracellular region of EPOR is capable of binding to EPO.

16. The T cell of any one of claims 1 to 15, wherein the recombinant protein comprises an endodomain.

17. The T cell of any one of claims 1 to 15, wherein the recombinant protein is capable of associating with a signalling protein comprising an endodomain.

18. The T cell of claim 16 or 17, wherein the endodomain comprises a tyrosine kinase activating domain comprising a JAK1 and/or a JAK2 binding motif and a tyrosine effector domain comprising one or more tyrosine residues that can be phosphorylated by JAK1 and/or JAK2.

19. The T cell of claim 18, wherein the tyrosine effector domain comprises at least one STAT association motif, preferably a STAT5 association motif.

20. The T cell of any one of claims 16 to 19, wherein the endodomain further comprises a JAK3 binding motif.

21. The T cell of any one of claims 16 to 20, wherein the endodomain comprises or consists of an EPOR endodomain as set out in SEQ ID NO. 8 or an EPOR endodomain variant having at least 40% sequence identity to SEQ ID NO. 8.

22. The T cell of claim 21, wherein the EPOR endodomain variant comprises at least one modification to reduce the binding of SHP1 to the EPOR endodomain sequence, particularly wherein the modification is to Y181 and/or Y183 of SEQ ID NO. 8.

23. The T cell of claim 21 or 22, wherein the EPOR endodomain variant comprises or consists of a truncated EPOR endodomain truncating at least the portion of the EPOR endodomain comprising Y181 and/or Y183 of SEQ ID NO. 8, for example wherein the truncated EPOR endodomain comprises or consists of the sequence of SEQ ID NO. 62, SEQ ID NO. 106 or SEQ ID NO. 107 or a variant thereof having at least 80% sequence identity thereto.

24. The T cell of any one of claims 21 to 23, wherein the variant EPOR endodomain variant comprises an insertion of at least the sequence of SEQ ID NO. 108 or a sequence differing to SEQ ID NO. 8 by no more than two amino acids, for example wherein the variant comprises an insertion of at least the sequence of SEQ ID NO. 109, 110, 111, 112, or 113 or a sequence differing to SEQ ID NO. 109, 110, 111, 112, or 113 respectively by no more than one or no more than two amino acids.

25. The T cell of claim 24, wherein the EPOR endodomain variant comprises or consists of the sequence of SEQ ID NO. 114, SEQ ID NO. 115 or SEQ ID NO. 116, or a variant thereof having at least 80% sequence identity thereto.

26. A T cell comprising a nucleic acid molecule comprising a nucleotide sequence encoding a recombinant protein as defined in any one of claims 1 to 25.

27. A T cell comprising a construct comprising a nucleic acid molecule as defined in claim 26 and one or more further nucleotide sequences.

28. The T cell of claim 27, wherein the further nucleotide sequence: (i) is a regulatory sequence; and/or (ii) encodes a protein of interest; and/or wherein the protein of interest is (iii) a therapeutic protein, (iv) an antigen receptor, (v) a CAR or TCR, (vi) a safety switch polypeptide or (vii) a FOXP3 polypeptide.

29. A T cell comprising a vector comprising a nucleic acid molecule or construct as defined in claims 26 to 28.

30. A cell population comprising a T cell as defined in any one of claims 1 to 29.

31. A method for making a T cell according to any one of claims 1 to 29 which comprises the step of introducing into the cell a nucleic acid molecule, construct or vector as defined in any one of claims 26 to 29.

32. A method of promoting the survival or persistence of a cell, said method comprising introducing into the cell, a nucleic acid molecule, construct or vector as defined in any one of claims 26 to 29.

33. The method of claim 32, further comprising the step of culturing said cell in the presence of EPO.

34. A pharmaceutical composition comprising a cell or cell population as defined in any one of claims 1 to 30.

35. A T cell or cell population of anyone of claims 1 to 30 or a pharmaceutical composition of claim 34 for use in therapy.

36. A T cell or cell population of anyone of claims 1 to 30 or a pharmaceutical composition of claim 34 for treating cancer, an infectious, neurodegenerative, or inflammatory disease, or for inducing immunosuppression.

37. The T cell, cell population or pharmaceutical composition of claim 36 for use in induction of tolerance to a transplant; treating and/or preventing graft-versus-host disease (GvHD), an autoimmune or allergic disease; to promote tissue repair and/or tissue regeneration; or to ameliorate inflammation in a subject, preferably wherein the cell is a Treg cell.


SEQUENCE LISTING (116 sequences)

SEQ ID NO: 1 - protein, 508 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 2 - protein, 226 aa, synthetic construct
APPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVACLADESGHVVLRWLPPPETPMTSHIRYEVDVSAGNGAGSVQRVEILEGRTECVLSNLRGRTRYTFAVRARMAEPSFGGFWSAWSEPVSLLTPSDLDP

SEQ ID NO: 3 - protein, 226 aa, Homo sapiens
APPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDP

SEQ ID NO: 4 - protein, 250 aa, synthetic construct
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVACLADESGHVVLRWLPPPETPMTSHIRYEVDVSAGNGAGSVQRVEILEGRTECVLSNLRGRTRYTFAVRARMAEPSFGGFWSAWSEPVSLLTPSDLDP

SEQ ID NO: 5 - protein, 250 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDP

SEQ ID NO: 6 - protein, 24 aa, Homo sapiens
MDHLGASLWPQVGSLCLLLAGAAW

SEQ ID NO: 7 - protein, 23 aa, Homo sapiens
LILTLSLILVVILVLLTVLALLS

SEQ ID NO: 8 - protein, 235 aa, Homo sapiens
HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 9 - protein, 507 aa, Mus sp.
MDKLRVPLWPRVGPLCLLLAGAAWAPSPSLPDPKFESKAALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSHRRTLQQKIWPGIPSPESEFEGLFTTHKGNFQLWLLQRDGCLWWSPGSSFPEDPPAHLEVLSEPRWAVTQAGDPGADDEGPLLEPVGSEHAQDTYLVLDKWLLPRTPCSENLSGPGGSVDPVTMDEASETSSCPSDLASKPRPEGTSPSSFEYTILDPSSQLLCPRALPPELPPTPPHLKYLYLVVSDSGISTDYSSGGSQGVHGDSSDGPYSHPYENSLVPDSEPLHPGYVACS

SEQ ID NO: 10 - protein, 249 aa, synthetic construct
MDKLRVPLWPRVGPLCLLLAGAAWAPSPSLPDPKFESKAALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLACRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDP

SEQ ID NO: 11 - protein, 249 aa, Mus sp.
MDKLRVPLWPRVGPLCLLLAGAAWAPSPSLPDPKFESKAALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDP

SEQ ID NO: 12 - protein, 23 aa, Mus sp.
LILTLSLILVLISLLLTVLALLS

SEQ ID NO: 13 - protein, 235 aa, Mus sp.
HRRTLQQKIWPGIPSPESEFEGLFTTHKGNFQLWLLQRDGCLWWSPGSSFPEDPPAHLEVLSEPRWAVTQAGDPGADDEGPLLEPVGSEHAQDTYLVLDKWLLPRTPCSENLSGPGGSVDPVTMDEASETSSCPSDLASKPRPEGTSPSSFEYTILDPSSQLLCPRALPPELPPTPPHLKYLYLVVSDSGISTDYSSGGSQGVHGDSSDGPYSHPYENSLVPDSEPLHPGYVACS

SEQ ID NO: 14 - protein, 193 aa, Homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 15 - protein, 108 aa, Homo sapiens
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 16 - protein, 100 aa, Homo sapiens
MASRILWHEMWHEGLEEASRLYFGERNVKGMFEVLEPLHAMMERGPQTLKETSFNQAYGRDLMEAQEWCRKYMKSGNVKDLTQAWDLYYHVFRRISKLES

SEQ ID NO: 17 - protein, 100 aa, synthetic construct
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKLES

SEQ ID NO: 18 - protein, 100 aa, synthetic construct
MASRILWHEMWHEGLEEASRLYFGERNVKGMFEVLEPLHAMMERGPQTLKETSFNQAYGRDLMEAQEWCRKYMKSGNVKDLHQAFDLYYHVFRRISKLES

SEQ ID NO: 19 - protein, 100 aa, synthetic construct
MASRILWHEMWHEGLEEASRLYFGERNVKGMFEVLEPLHAMMERGPQTLKETSFNQAYGRDLMEAQEWCRKYMKSGNVPDLTQAWDLYYHVFRRISKLES

SEQ ID NO: 20 - protein, 39 aa, Homo sapiens
RIARLEEKVKTLKAQNSELASTANMLREQVAQLKQKVMN

SEQ ID NO: 21 - protein, 40 aa, synthetic construct
LTDTLQAETDQLEDKKSALQTEIANLLKEKEKLEFILAAY

SEQ ID NO: 22 - protein, 51 aa, synthetic construct
MDPDLEIRAAFLRQRNTALRTEVAELEQEVQRLENEVSQYETRYGPLGGGK

SEQ ID NO: 23 - protein, 51 aa, synthetic construct
MDPDLEIEAAFLERENTALETRVAELRQRVQRLRNRVSQYRTRYGPLGGGK

SEQ ID NO: 24 - protein, 27 aa, Homo sapiens
FWVLVVVGGVLACYSLLVTVAFIIFWV

SEQ ID NO: 25 - protein, 25 aa, Homo sapiens
IPWLGHLLVGLSGAFGFIILVYLLI

SEQ ID NO: 26 - protein, 21 aa, Homo sapiens
IYIWAPLAGTCGVLLLSLVIT

SEQ ID NO: 27 - protein, 21 aa, Homo sapiens
IVILLAGGFLSKSLVFSVLFA

SEQ ID NO: 28 - protein, 21 aa, Homo sapiens
ILLLLACIFLIKILAASALWA

SEQ ID NO: 29 - protein, 21 aa, synthetic construct
LLAGLVAANAVASLLIVGAVF

SEQ ID NO: 30 - protein, 21 aa, synthetic construct
GVLAGIVMGNLVLTVLIALAV

SEQ ID NO: 31 - protein, 286 aa, Homo sapiens
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 32 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 33 - protein, 230 aa, Homo sapiens
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 34 - protein, 569 aa, Homo sapiens
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 35 - protein, 437 aa, Homo sapiens
RFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 36 - protein, 189 aa, Homo sapiens
RHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 37 - protein, 362 aa, synthetic construct
GGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEF

SEQ ID NO: 38 - skipped

SEQ ID NO: 39 - protein, 4 aa, synthetic construct
YCTF

SEQ ID NO: 40 - protein, 4 aa, synthetic construct
YFFF

SEQ ID NO: 41 - protein, 4 aa, synthetic construct
YLSL

SEQ ID NO: 42 - protein, 5 aa, synthetic construct
YLSLQ

SEQ ID NO: 43 - protein, 57 aa, synthetic construct
KVLKCNTPDPSKFFSQLSSEHGGDVQKWLSSPFPSSSFSPGGLAPEISPLEVLERDK

SEQ ID NO: 44 - protein, 45 aa, synthetic construct
NPWFQRAKMPRALDFSGHTHPVATFQPSRPESVNDLFLCPQKELT

SEQ ID NO: 45 - protein, 42 aa, synthetic construct
GYICLRNSLPKVLNFHNFLAWPFPNLPPLEAMDMVEVIYINR

SEQ ID NO: 46 - protein, 42 aa, synthetic construct
PLKEKSIILPKSLISVVRSATLETKPESKYVSLITSYQPFSL

SEQ ID NO: 47 - protein, 43 aa, synthetic construct
RRRKKLPSVLLFKKPSPFIFISQRPSPETQDTIHPLDEEAFLK

SEQ ID NO: 48 - protein, 45 aa, synthetic construct
YIHVGKEKHPANLILIYGNEFDKRFFVPAEKIVINFITLNISDDS

SEQ ID NO: 49 - protein, 41 aa, synthetic construct
RYVTKPPAPPNSLNVQRVLTFQPLRFIQEHVLIPVFDLSGP

SEQ ID NO: 50 - protein, 43 aa, synthetic construct
NYVFFPSLKPSSSIDEYFSEQPLKNLLLSTSEEQIEKCFIIEN

SEQ ID NO: 51 - protein, 49 aa, synthetic construct
YWFHTPPSIPLQIEEYLKDPTQPILEALDKDSSPKDDVWDSVSIISFPE

SEQ ID NO: 52 - protein, 51 aa, synthetic construct
YAFSPRNSLPQHLKEFLGHPHHNTLLFFSFPLSDENDVFDKLSVIAEDSES

SEQ ID NO: 53 - protein, 94 aa, synthetic construct
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 54 - protein, 208 aa, synthetic construct
NCRNTGPWLKKVLKCNTPDPSKFFSQLSSEHGGDVQKWLSSPFPSSSFSPGGLAPEISPLEVLERDKVTQLLDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 55 - protein, 52 aa, synthetic construct
ERTMPRIPTLKNLEDLVTEYHGNFSAWSGVSKGLAESLQPDYSERLCLVSEI

SEQ ID NO: 56 - protein, 86 aa, synthetic construct
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 57 - skipped

SEQ ID NO: 58 - protein, 4 aa, synthetic construct
YRHQ

SEQ ID NO: 59 - protein, 4 aa, synthetic construct
YLRQ

SEQ ID NO: 60 - protein, 31 aa, synthetic construct
QLLLQQDKVPEPASLSSNHSLTSCFTNQGYF

SEQ ID NO: 61 - protein, 595 aa, synthetic construct
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 62 - protein, 180 aa, synthetic construct
HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLK

SEQ ID NO: 63 - protein, 52 aa, synthetic construct
IESVLCLRESYDPQLSEALGKSVGSWASFNGHYETVLDELNKLTPIRPMTRE

SEQ ID NO: 64 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 65 - protein, 41 aa, Homo sapiens
RSKRSRLLHSDYMNMTPRRPGPTRKHYQPYAPPRDFAAYRS

SEQ ID NO: 66 - protein, 48 aa, Homo sapiens
QRRKYRSNKGESPVEPAEPCHYSCPREEEGSTIPIQEDYRKPEPACSP

SEQ ID NO: 67 - protein, 4 aa, synthetic construct
GGGS

SEQ ID NO: 68 - protein, 10 aa, synthetic construct
ETSGGGGSRL

SEQ ID NO: 69 - protein, 16 aa, synthetic construct
SGGGGSGGGGSGGGGS

SEQ ID NO: 70 - protein, 5 aa, synthetic construct
GGGGS

SEQ ID NO: 71 - protein, 11 aa, synthetic construct
SGGGGSGGGGS

SEQ ID NO: 72 - protein, 21 aa, synthetic construct
SGGGGSGGGGSGGGGSGGGGS

SEQ ID NO: 73 - protein, 26 aa, synthetic construct
SGGGGSGGGGSGGGGSGGGGSGGGGS

SEQ ID NO: 74 - protein, 6 aa, synthetic construct
GGGGGS

SEQ ID NO: 75 - protein, 7 aa, synthetic construct
GGGGGGS

SEQ ID NO: 76 - protein, 6 aa, synthetic construct
GGGGGG

SEQ ID NO: 77 - protein, 8 aa, synthetic construct
GGGGGGGG

SEQ ID NO: 78 - protein, 18 aa, synthetic construct
KESGSVSSEQLAQFRSLD

SEQ ID NO: 79 - protein, 14 aa, synthetic construct
EGKSSGSGSESKST

SEQ ID NO: 80 - protein, 12 aa, synthetic construct
GSAGSAAGSGEF

SEQ ID NO: 81 - protein, 16 aa, synthetic construct
SGGGGSAGSAAGSGEF

SEQ ID NO: 82 - protein, 16 aa, synthetic construct
SGGGLLLLLLLLGGGS

SEQ ID NO: 83 - protein, 16 aa, synthetic construct
SGGGAAAAAAAAGGGS

SEQ ID NO: 84 - protein, 24 aa, synthetic construct
SGGGAAAAAAAAAAAAAAAAGGGS

SEQ ID NO: 85 - protein, 24 aa, synthetic construct
SGALGGLALAGLLLAGLGLGAAGS

SEQ ID NO: 86 - protein, 14 aa, synthetic construct
SLSLSPGGGGGPAR

SEQ ID NO: 87 - protein, 25 aa, synthetic construct
SLSLSPGGGGGPARSLSLSPGGGGG

SEQ ID NO: 88 - protein, 6 aa, synthetic construct
GSSGSS

SEQ ID NO: 89 - protein, 7 aa, synthetic construct
GSSSSSS

SEQ ID NO: 90 - protein, 6 aa, synthetic construct
GGSSSS

SEQ ID NO: 91 - protein, 6 aa, synthetic construct
GSSSSS

SEQ ID NO: 92 - protein, 6 aa, synthetic construct
SGGGGS

SEQ ID NO: 93 - protein, 15 aa, synthetic construct
GGGGSGGGGSGGGGS

SEQ ID NO: 94 - protein, 5 aa, synthetic construct
GGGGG

SEQ ID NO: 95 - protein, 10 aa, synthetic construct
GGGGSGGGGS

SEQ ID NO: 96 - protein, 20 aa, synthetic construct
GGGGSGGGGSGGGGSGGGGS

SEQ ID NO: 97 - protein, 7 aa, synthetic construct
GGGGGGG

SEQ ID NO: 98 - protein, 9 aa, synthetic construct
GGGGGGGGG

SEQ ID NO: 99 - protein, 137 aa, synthetic construct
ACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 100 - protein, 19 aa, synthetic construct
ATNFSLLKQAGDVEENPGP

SEQ ID NO: 101 - protein, 18 aa, synthetic construct
EGRGSLLTCGDVEENPGP

SEQ ID NO: 102 - protein, 20 aa, synthetic construct
QCTNYALLKLAGDVESNPGP

SEQ ID NO: 103 - protein, 22 aa, synthetic construct
VKQTLNFDLLKLAGDVESNPGP

SEQ ID NO: 104 - skipped

SEQ ID NO: 105 - protein, 4 aa, synthetic construct
RRKR

SEQ ID NO: 106 - protein, 105 aa, Homo sapiens
HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPR

SEQ ID NO: 107 - protein, 160 aa, Homo sapiens
HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSS

SEQ ID NO: 108 - protein, 5 aa, synthetic construct
MDTVP

SEQ ID NO: 109 - protein, 6 aa, synthetic construct
SMDTVP

SEQ ID NO: 110 - protein, 7 aa, synthetic construct
ASMDTVP

SEQ ID NO: 111 - protein, 8 aa, synthetic construct
LASMDTVP

SEQ ID NO: 112 - protein, 9 aa, synthetic construct
ALASMDTVP

SEQ ID NO: 113 - protein, 10 aa, synthetic construct
PALASMDTVP

SEQ ID NO: 114 - protein, 245 aa, synthetic construct
HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACSPALASMDTVP

SEQ ID NO: 115 - protein, 115 aa, synthetic construct
HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRPALASMDTVP

SEQ ID NO: 116 - protein, 170 aa, synthetic construct
HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSPALASMDTVP